SEQUENCE LISTING (65 sequences)

SEQ ID NO: 1 - protein, 8 aa, Artificial Sequence
  peptide
SREKKAAK

SEQ ID NO: 2 - protein, 55 aa, Artificial Sequence
  derived human beta-2 adrenergic GPCR
RVFQEAKRQLQKIDKSEGRFHVQNLSQVEQDGRTGHGLRRSSKFCSREKKAAKTL

SEQ ID NO: 3 - protein, 55 aa, Artificial Sequence
  derived from cat beta-2 adrenergic GPCR
RVFQVAQRQLQKIDKSEGRFHAQNLSQVEQDGRSGHGHRRASKFCSREKKAAKTL

SEQ ID NO: 4 - protein, 55 aa, Artificial Sequence
  derived from dog beta-2 adrenergic GPCR
RVFQVAQRQLQKIDRSEGRFHAQNLSQVEQDGRSGHGHRRSSKFCSREKKAAKTL

SEQ ID NO: 5 - protein, 55 aa, Artificial Sequence
  derived from guinea pig beta-2 adrenergic GPCR
RVFQVAKKQLQKIDRSEGRFHTQNLSQVEQDGRSGHGLRRSSKFYSREKKAAKTL

SEQ ID NO: 6 - protein, 55 aa, Artificial Sequence
  derived from mouse beta-2 adrenergic GPCR
RVFQVAKRQLQKIDKSEGRFHAQNLSQVEQDGRTGHGLRRSSKFCSREKKAAKTL

SEQ ID NO: 7 - protein, 55 aa, Artificial Sequence
  derived from rat beta-2 adrenergic GPCR
RVFQVAKRQLQKIDKSEGRFHAQNLSQVEQDGRSGHGLRSSSKFCSREKKAAKTL

SEQ ID NO: 8 - protein, 55 aa, Artificial Sequence
  derived from rhesus monkey beta-2 adrenergic GPCR
RVFQEAKRQLQKIDKSEGRFHAQNLSQVEQDGRTGHGLRRSSKFCSREKKAAKTL

SEQ ID NO: 9 - DNA, 177 bp, Artificial Sequence
  polynucleotide with flanking restriction sites
ggtaccagggtctttcaggaggccaaaaggcagctccagaagattgacaaatctgagggccgcttccatgtccagaaccttagccaggtggagcaggatgggcggacggggcatggactccgcagatcttccaagttctgctccagggaaaagaaagcagccaaaaccttggaattc

SEQ ID NO: 10 - protein, 717 aa, Artificial Sequence
  derived from rat cholinergic receptor, muscarinic 3
MTLHSNSTTSPLFPNISSSWVHSPSEAGLPLGTVTQLGSYNISQETGNFSSNDTSSDPLGGHTIWQVVFIAFLTGFLALVTIIGNILVIVAFKVNKQLKTVNNYFLLSLACADLIIGVISMNLFTTYIIMNRWALGNLACDLWLSIDYVASNASVMNLLVISFDRYFSITRPLTYRAKRTTKRRGVMIGLAWVISFVLWAPAILFWQYFVGKRTVPPGECFIQFLSEPTITFGTAIAAFYMPVTIMTILGTRVFQEAKRQLQKIDKSEGRFHVQNLSQVEQDGRTGHGLRRSSKFCSREKKAAKTLEFILLAFIITWTPYNIMVLVNTFCDSCIPKTYWNLGYWLCYINSTVNPVCYALCNKTFRTTFKTLLLCQCDKRKRRKQQYQQRQSVIFHKRVPEQALENSMTSKVYDPEQRKRMITGPQWWARCKQMNVLDSFINYYDSEKHAENAVIFLHGNAASSYLWRHVVPHIEPVARCIIPDLIGMGKSGKSGNGSYRLLDHYKYLTAWFELLNLPKKINFVGHDWGACLAFHYSYEHQDKIKAIVHAESVVDVIESWDEWPDIEEDIALIKSEEGEKMVLENNFFVETMLPSKIMRKLEPEEFAAYLEPFKEKGEVRRPTLSWPREIPLVKGGKPDVVQIVRNYNAYLRASDDLPKMFIESDPGFFSNAIVEGAKKFPNTEFVKVKGLHFSQEDAPDEMGKYIKSFVERVLKNEQ

SEQ ID NO: 11 - protein, 676 aa, Artificial Sequence [sequence omitted]

SEQ ID NO: 12 - protein, 662 aa, Artificial Sequence
  derived from human cholinergic receptor, muscarinic 4
MANFTPVNGSSGNQSVRLVTSSSHNRYETVEMVFIATVTGSLSLVTVVGNILVMLSIKVNRQLQTVNNYFLFSLACADLIIGAFSMNLYTVYIIKGYWPLGAVVCDLWLALDYVVSNASVMNLLIISFDRYFCVTKPLTYPARRTTKMAGLMIAAAWVLSFVLWAPAILFWQFVVGKRTVPDNQCFIQFLSNPAVTFGTAIAAFYLPVVIMTVLGTRVFQEAKRQLQKIDKSEGRFHVQNLSQVEQDGRTGHGLRRSSKFCSREKKAAKTLEFILLAFILTWTPYNVMVLVNTFCQSCIPDTVWSIGYWLCYVNSTINPACYALCNATFKKTFRHLLLCQYRNIGTARENSMTSKVYDPEQRKRMITGPQWWARCKQMNVLDSFINYYDSEKHAENAVIFLHGNAASSYLWRHVVPHIEPVARCIIPDLIGMGKSGKSGNGSYRLLDHYKYLTAWFELLNLPKKINFVGHDWGACLAFHYSYEHQDKIKAIVHAESVVDVIESWDEWPDIEEDIALIKSEEGEKMVLENNFFVETMLPSKIMRKLEPEEFAAYLEPFKEKGEVRRPTLSWPREIPLVKGGKPDVVQIVRNYNAYLRASDDLPKMFIESDPGFFSNAIVEGAKKFPNTEFVKVKGLHFSQEDAPDEMGKYIKSFVERVLKNEQ

SEQ ID NO: 13 - protein, 708 aa, Artificial Sequence [sequence omitted]

SEQ ID NO: 14 - protein, 649 aa, Artificial Sequence [sequence omitted]

SEQ ID NO: 15 - protein, 705 aa, Artificial Sequence
  derived from human ghrelin receptor
MWNATPSEEPGFNLTLADLDWDASPGNDSLGDELLQLFPAPLLAGVTATCVALFVVGIAGNLLTMLVVSRFRELRTTTNLYLSSMAFSDLLIFLCMPLDLVRLWQYRPWNFGDLLCKLFQFVSESCTYATVLTITALSVERYFAICFPLRAKVVVTKGRVKLVIFVIWAVAFCSAGPIFVLVGVEHENGTDPWDTNECRPTEFAVRSGLLTVMVWVSSIFFFLPVFCLTVLGTRVFQEAKRQLQKIDKSEGRFHVQNLSQVEQDGRTGHGLRRSSKFCSREKKAAKTLEFAVVVFAFILCWLPFHVGRYLFSKSFEPGSLEIAQISQYCNLVSFVLFYLSAAINPILYNIMSKKYRVAVFRLLGFEPFSQRKLSTLKDESSRAWTESSINTENSMTSKVYDPEQRKRMITGPQWWARCKQMNVLDSFINYYDSEKHAENAVIFLHGNAASSYLWRHVVPHIEPVARCIIPDLIGMGKSGKSGNGSYRLLDHYKYLTAWFELLNLPKKINFVGHDWGACLAFHYSYEHQDKIKAIVHAESVVDVIESWDEWPDIEEDIALIKSEEGEKMVLENNFFVETMLPSKIMRKLEPEEFAAYLEPFKEKGEVRRPTLSWPREIPLVKGGKPDVVQIVRNYNAYLRASDDLPKMFIESDPGFFSNAIVEGAKKFPNTEFVKVKGLHFSQEDAPDEMGKYIKSFVERVLKNEQ

SEQ ID NO: 16 - protein, 716 aa, Artificial Sequence
  derived from human CXCR3 chemokine receptor
MVLEVSDHQVLNDAEVAALLENFSSSYDYGENESDSCCTSPPCPQDFSLNFDRAFLPALYSLLFLLGLLGNGAVAAVLLSRRTALSSTDTFLLHLAVADTLLVLTLPLWAVDAAVQWVFGSGLCKVAGALFNINFYAGALLLACISFDRYLNIVHATQLYRRGPPARVTLTCLAVWGLCLLFALPDFIFLSAHHDERLNATHCQYNFPQVGRTALRVLQLVAGFLLPLLVMAYCGTRVFQEAKRQLQKIDKSEGRFHVQNLSQVEQDGRTGHGLRRSSKFCSREKKAAKTLEFVVVAFALCWTPYHLVVLVDILMDLGALARNCGRESRVDVAKSVTSGLGYMHCCLNPLLYAFVGVKFRERMWMLLLRLGCPNQRGLQRQPSSSRRDSSWSETSEASYSGLENSMTSKVYDPEQRKRMITGPQWWARCKQMNVLDSFINYYDSEKHAENAVIFLHGNAASSYLWRHVVPHIEPVARCIIPDLIGMGKSGKSGNGSYRLLDHYKYLTAWFELLNLPKKINFVGHDWGACLAFHYSYEHQDKIKAIVHAESVVDVIESWDEWPDIEEDIALIKSEEGEKMVLENNFFVETMLPSKIMRKLEPEEFAAYLEPFKEKGEVRRPTLSWPREIPLVKGGKPDVVQIVRNYNAYLRASDDLPKMFIESDPGFFSNAIVEGAKKFPNTEFVKVKGLHFSQEDAPDEMGKYIKSFVERVLKNEQ

SEQ ID NO: 17 - protein, 749 aa, Artificial Sequence [sequence omitted]

SEQ ID NO: 18 - protein, 770 aa, Artificial Sequence [sequence omitted]

SEQ ID NO: 19 - protein, 762 aa, Artificial Sequence [sequence omitted]

SEQ ID NO: 20 - protein, 749 aa, Artificial Sequence [sequence omitted]

SEQ ID NO: 21 - protein, 647 aa, Artificial Sequence
  derived from human dopamine receptor D3
MASLSQLSSHLNYTCGAENSTGASQARPHAYYALSYCALILAIVFGNGLVCMAVLKERALQTTTNYLVVSLAVADLLVATLVMPWVVYLEVTGGVWNFSRICCDVFVTLDVMMCTASILNLCAISIDRYTAVVMPVHYQHGTGQSSCRRVALMITAVWVLAFAVSCPLLFGFNTTGDPTVCSISNPDFVIYSSVVSFYLPFGVTVLVGTRVFQEAKRQLQKIDKSEGRFHVQNLSQVEQDGRTGHGLRRSSKFCSREKKAAKTLEFVLGAFIVCWLPFFLTHVLNTHCQTCHVSPELYSATTWLGYVNSALNPVIYTTFNIEFRKAFLKILSCENSMTSKVYDPEQRKRMITGPQWWARCKQMNVLDSFINYYDSEKHAENAVIFLHGNAASSYLWRHVVPHIEPVARCIIPDLIGMGKSGKSGNGSYRLLDHYKYLTAWFELLNLPKKINFVGHDWGACLAFHYSYEHQDKIKAIVHAESVVDVIESWDEWPDIEEDIALIKSEEGEKMVLENNFFVETMLPSKIMRKLEPEEFAAYLEPFKEKGEVRRPTLSWPREIPLVKGGKPDVVQIVRNYNAYLRASDDLPKMFIESDPGFFSNAIVEGAKKFPNTEFVKVKGLHFSQEDAPDEMGKYIKSFVERVLKNEQ

SEQ ID NO: 22 - protein, 655 aa, Artificial Sequence [sequence omitted]

SEQ ID NO: 23 - protein, 756 aa, Artificial Sequence [sequence omitted]

SEQ ID NO: 24 - protein, 695 aa, Artificial Sequence [sequence omitted]

SEQ ID NO: 25 - protein, 673 aa, Artificial Sequence
  derived from human histamine receptor H3
MERAPPDGPLNASGALAGDAAAAGGARGFSAAWTAVLAALMALLIVATVLGNALVMLAFVADSSLRTQNNFFLLNLAISDFLVGAFCIPLYVPYVLTGRWTFGRGLCKLWLVVDYLLCTSSAFNIVLISYDRFLSVTRAVSYRAQQGDTRRAVRKMLLVWVLAFLLYGPAILSWEYLSGGSSIPEGHCYAEFFYNWYFLITASTLEFFTPFLSVTFFGTRVFQEAKRQLQKIDKSEGRFHVQNLSQVEQDGRTGHGLRRSSKFCSREKKAAKTLEFIVSIFGLCWAPYTLLMIIRAACHGHCVPDYWYETSFWLLWANSAVNPVLYPLCHHSFRRAFTKLLCPQKLKIQPHSSLEHCWKENSMTSKVYDPEQRKRMITGPQWWARCKQMNVLDSFINYYDSEKHAENAVIFLHGNAASSYLWRHVVPHIEPVARCIIPDLIGMGKSGKSGNGSYRLLDHYKYLTAWFELLNLPKKINFVGHDWGACLAFHYSYEHQDKIKAIVHAESVVDVIESWDEWPDIEEDIALIKSEEGEKMVLENNFFVETMLPSKIMRKLEPEEFAAYLEPFKEKGEVRRPTLSWPREIPLVKGGKPDVVQIVRNYNAYLRASDDLPKMFIESDPGFFSNAIVEGAKKFPNTEFVKVKGLHFSQEDAPDEMGKYIKSFVERVLKNEQ

SEQ ID NO: 26 - protein, 660 aa, Artificial Sequence
  derived from pig adrenergic receptor, alpha-2A
MGSLQPEAGNASWNGTEAPGGGARATPYSLQVTLTLVCLAGLLMLFTVFGNVLVIIAVFTSRALKAPQNLFLVSLASADILVATLVIPFSLANEVMGYWYFGKAWCEIYLALDVLFCTSSIVHLCAISLDRYWSITQAIEYNLKRTPRRIKAIIVTVWVISAVISFPPLISIEKKAGGGGQQPAEPRCEINDQKWYVISSCIGSFFAPCLIMILVGTRVFQEAKRQLQKIDKSEGRFHVQNLSQVEQDGRTGHGLRRSSKFCSREKKAAKTLEFVIGVFVVCWFPFFFTYTLTAVGCSVPPTLFKFFFWFGYCNSSLNPVIYTIFNHDFRRAFKKILCRGDRKRIVENSMTSKVYDPEQRKRMITGPQWWARCKQMNVLDSFINYYDSEKHAENAVIFLHGNAASSYLWRHVVPHIEPVARCIIPDLIGMGKSGKSGNGSYRLLDHYKYLTAWFELLNLPKKINFVGHDWGACLAFHYSYEHQDKIKAIVHAESVVDVIESWDEWPDIEEDIALIKSEEGEKMVLENNFFVETMLPSKIMRKLEPEEFAAYLEPFKEKGEVRRPTLSWPREIPLVKGGKPDVVQIVRNYNAYLRASDDLPKMFIESDPGFFSNAIVEGAKKFPNTEFVKVKGLHFSQEDAPDEMGKYIKSFVERVLKNEQ

SEQ ID NO: 27 - protein, 690 aa, Artificial Sequence [sequence omitted]

SEQ ID NO: 28 - protein, 720 aa, Artificial Sequence [sequence omitted]

SEQ ID NO: 29 - protein, 702 aa, Artificial Sequence [sequence omitted]

SEQ ID NO: 30 - protein, 739 aa, Artificial Sequence [sequence omitted]

SEQ ID NO: 31 - protein, 673 aa, Artificial Sequence [sequence omitted]

SEQ ID NO: 32 - protein, 818 aa, Artificial Sequence [sequence omitted]

SEQ ID NO: 33 - protein, 660 aa, Artificial Sequence [sequence omitted]

SEQ ID NO: 34 - protein, 789 aa, Artificial Sequence [sequence omitted]

SEQ ID NO: 35 - protein, 693 aa, Artificial Sequence [sequence omitted]

SEQ ID NO: 36 - protein, 646 aa, Artificial Sequence
  derived from human GPR40 (insulin secretion)
MDLPPQLSFGLYVAAFALGFPLNVLAIRGATAHARLRLTPSLVYALNLGCSDLLLTVSLPLKAVEALASGAWPLPASLCPVFAVAHFFPLYAGGGFLAALSAGRYLGAAFPLGYQAFRRPCYSWGVCAAIWALVLCHLGLVFGLEAPGGWLDHSNTSLGINTPVNGSPVCLEAWDPASAGPARFSLSLLLFFLPLAITAFCGTRVFQEAKRQLQKIDKSEGRFHVQNLSQVEQDGRTGHGLRRSSKFCSREKKAAKTLEFALLTLLLCVGPYNASNVASFLYPNLGGSWRKLGLITGAWSVVLNPLVTGYLGRGPGLKTVCAARTQGGKSQKENSMTSKVYDPEQRKRMITGPQWWARCKQMNVLDSFINYYDSEKHAENAVIFLHGNAASSYLWRHVVPHIEPVARCIIPDLIGMGKSGKSGNGSYRLLDHYKYLTAWFELLNLPKKINFVGHDWGACLAFHYSYEHQDKIKAIVHAESVVDVIESWDEWPDIEEDIALIKSEEGEKMVLENNFFVETMLPSKIMRKLEPEEFAAYLEPFKEKGEVRRPTLSWPREIPLVKGGKPDVVQIVRNYNAYLRASDDLPKMFIESDPGFFSNAIVEGAKKFPNTEFVKVKGLHFSQEDAPDEMGKYIKSFVERVLKNEQ

SEQ ID NO: 37 - protein, 662 aa, Artificial Sequence [sequence omitted]

SEQ ID NO: 38 - DNA, 2154 bp, Artificial Sequence
  Modified rat cholinergic receptor, muscarinic 3
atgaccttgcacagtaacagtacaacctcgcctttgtttcccaacatcagctcttcctgggtgcacagtccctcggaggcagggctgcccttggggacagtcactcagttgggcagctacaacatttcacaagaaactgggaatttctcctcaaacgacacctccagcgaccctctcgggggtcacaccatctggcaagtggtcttcattgccttcttaactggcttcctggcattggtgaccatcattggcaacatccttgtcattgtggccttcaaggtcaacaaacagctgaagacagtcaacaactacttcctcttaagcctggcctgtgcagacctgatcatcggggtcatttccatgaacctgttcactacctacatcattatgaaccgttgggcactggggaacttagcctgcgacctctggctctccattgactatgtggccagcaatgcctctgtcatgaatctgctggtcatcagctttgacaggtacttttccatcactaggccactcacctaccgagccaaaagaacaacaaaacgacgtggtgtgatgattggtctggcttgggtcatctcctttgtcctatgggctcctgccatcttgttctggcaatactttgtagggaagagaactgtgcccccaggagaatgtttcattcagtttctgagtgagcccaccatcaccttcggcacggcgatcgctgccttttacatgcctgtcaccatcatgactattttaggtaccagggtctttcaggaggccaaaaggcagctccagaagattgacaaatctgagggccgcttccatgtccagaaccttagccaggtggagcaggatgggcggacggggcatggactccgcagatcttccaagttctgctccagggaaaagaaagcagccaaaaccttggaattcatcttgctagccttcatcatcacgtggaccccctacaacatcatggtcctggtgaacaccttctgtgacagctgcatacccaaaacctattggaatctgggctactggctgtgctatatcaacagcaccgtgaaccctgtgtgctatgccctgtgcaacaaaacattcagaaccaccttcaagacgctcctcttgtgccagtgtgacaaaaggaagaggcgcaaacagcagtaccagcagagacagtcggtcatttttcacaagcgagtgccggagcaggccttggagaattccatgacttcgaaagtttatgatccagaacaaaggaaacggatgataactggtccgcagtggtgggccagatgtaaacaaatgaatgttcttgattcatttattaattattatgattcagaaaaacatgcagaaaatgctgttatttttttacatggtaacgcggcctcttcttatttatggcgacatgttgtgccacatattgagccagtagcgcggtgtattataccagatcttattggtatgggcaaatcaggcaaatctggtaatggttcttataggttacttgatcattacaaatatcttactgcatggtttgaacttcttaatttaccaaagaagatcaattttgtcggccatgattggggtgcttgtttggcatttcattatagctatgagcatcaagataagatcaaagcaatagttcacgctgaaagtgtagtagatgtgattgaatcatgggatgaatggcctgatattgaagaagatattgcgttgatcaaatctgaagaaggagaaaaaatggttttggagaataacttcttcgtggaaaccatgttgccatcaaaaatcatgagaaagttagaaccagaagaatttgcagcatatcttgaaccattcaaagagaaaggtgaagttcgtcgtccaacattatcatggcctcgtgaaatcccgttagtaaaaggtggtaaacctgacgttgtacaaattgttaggaattataatgcttatctacgtgcaagtgatgatttaccaaaaatgtttattgaatcggatccaggattcttttccaatgctattgttgaaggcgccaagaagtttcctaatactgaatttgtcaaagtaaaaggtcttcatttttcgcaagaagatgcacctgatgaaatgggaaaatatatcaaatcgttcgttgagcgagttctcaaaaatgaacaataa

SEQ ID NO: 39 - DNA, 2031 bp, Artificial Sequence
  Modified human melanin-concentrating hormone receptor 2
atgaatccatttcatgcatcttgttggaacacctctgccgaacttttaaacaaatcctggaataaagagtttgcttatcaaactgccagtgtggtagatacagtcatcctcccttccatgattgggattatctgttcaacagggctggttggcaacatcctcattgtattcactataataagatccaggaaaaaaacagtccctgacatctatatctgcaacctggctgtggctgatttggtccacatagttggaatgccttttcttattcaccaatgggcccgagggggagagtgggtgtttggggggcctctctgcaccatcatcacatccctggatacttgtaaccaatttgcctgtagtgccatcatgactgtaatgagtgtggacaggtactttgccctcgtccaaccatttcgactgacacgttggagaacaaggtacaagaccatccggatcaatttgggcctttgggcagcttcctttatcctggcattgcctgtctgggtctactcgaaggtcatcaaatttaaagacggtgttgagagttgtgcttttgatttgacatcccctgacgatgtactctggtatacactttatttgacgataacaactttttttttccctctacccttgattttggtgtgctatattggtaccagggtctttcaggaggccaaaaggcagctccagaagattgacaaatctgagggccgcttccatgtccagaaccttagccaggtggagcaggatgggcggacggggcatggactccgcagatcttccaagttctgctccagggaaaagaaagcagccaaaaccttggaattcctggtggtagtctttatcctgagtgctgccccttatcatgtgatacaactggtgaacttacagatggaacagcccacactggccttctatgtgggttattacctctccatctgtctcagctatgccagcagcagcattaacccttttctctacatcctgctgagtggaaatttccagaaacgtctgcctcaaatccaaagaagagcgactgagaaggaaatcaacaatatgggaaacactctgaaatcacactttgagaattccatgacttcgaaagtttatgatccagaacaaaggaaacggatgataactggtccgcagtggtgggccagatgtaaacaaatgaatgttcttgattcatttattaattattatgattcagaaaaacatgcagaaaatgctgttatttttttacatggtaacgcggcctcttcttatttatggcgacatgttgtgccacatattgagccagtagcgcggtgtattataccagatcttattggtatgggcaaatcaggcaaatctggtaatggttcttataggttacttgatcattacaaatatcttactgcatggtttgaacttcttaatttaccaaagaagatcaattttgtcggccatgattggggtgcttgtttggcatttcattatagctatgagcatcaagataagatcaaagcaatagttcacgctgaaagtgtagtagatgtgattgaatcatgggatgaatggcctgatattgaagaagatattgcgttgatcaaatctgaagaaggagaaaaaatggttttggagaataacttcttcgtggaaaccatgttgccatcaaaaatcatgagaaagttagaaccagaagaatttgcagcatatcttgaaccattcaaagagaaaggtgaagttcgtcgtccaacattatcatggcctcgtgaaatcccgttagtaaaaggtggtaaacctgacgttgtacaaattgttaggaattataatgcttatctacgtgcaagtgatgatttaccaaaaatgtttattgaatcggatccaggattcttttccaatgctattgttgaaggcgccaagaagtttcctaatactgaatttgtcaaagtaaaaggtcttcatttttcgcaagaagatgcacctgatgaaatgggaaaatatatcaaatcgttcgttgagcgagttctcaaaaatgaacaataa

SEQ ID NO: 40 - DNA, 1989 bp, Artificial Sequence
  Modified human cholinergic receptor, muscarinic 4
atggccaacttcacacctgtcaatggcagctcgggcaatcagtccgtgcgcctggtcacgtcatcatcccacaatcgctatgagacggtggaaatggtcttcattgccacagtgacaggctccctgagcctggtgactgtcgtgggcaacatcctggtgatgctgtccatcaaggtcaacaggcagctgcagacagtcaacaactacttcctcttcagcctggcgtgtgctgatctcatcataggcgccttctccatgaacctctacaccgtgtacatcatcaagggctactggcccctgggcgccgtggtctgcgacctgtggctggccctggactacgtggtgagcaacgcctccgtcatgaaccttctcatcatcagctttgaccgctacttctgcgtcaccaagcctctcacctaccctgcccggcgcaccaccaagatggcaggcctcatgattgctgctgcctgggtactgtccttcgtgctctgggcgcctgccatcttgttctggcagtttgtggtgggtaagcggacggtgcccgacaaccagtgcttcatccagttcctgtccaacccagcagtgacctttggcacagccattgctgccttctacctgcctgtggtcatcatgacggtgctgggtaccagggtctttcaggaggccaaaaggcagctccagaagattgacaaatctgagggccgcttccatgtccagaaccttagccaggtggagcaggatgggcggacggggcatggactccgcagatcttccaagttctgctccagggaaaagaaagcagccaaaaccttggaattcattctgctagccttcatcctcacctggacgccctacaacgtcatggtcctggtgaacaccttctgccagagctgcatccctgacacggtgtggtccattggctactggctctgctacgtcaacagcaccatcaaccctgcctgctatgctctgtgcaacgccacctttaaaaagaccttccggcacctgctgctgtgccagtatcggaacatcggcactgccagggagaattccatgacttcgaaagtttatgatccagaacaaaggaaacggatgataactggtccgcagtggtgggccagatgtaaacaaatgaatgttcttgattcatttattaattattatgattcagaaaaacatgcagaaaatgctgttatttttttacatggtaacgcggcctcttcttatttatggcgacatgttgtgccacatattgagccagtagcgcggtgtattataccagatcttattggtatgggcaaatcaggcaaatctggtaatggttcttataggttacttgatcattacaaatatcttactgcatggtttgaacttcttaatttaccaaagaagatcaattttgtcggccatgattggggtgcttgtttggcatttcattatagctatgagcatcaagataagatcaaagcaatagttcacgctgaaagtgtagtagatgtgattgaatcatgggatgaatggcctgatattgaagaagatattgcgttgatcaaatctgaagaaggagaaaaaatggttttggagaataacttcttcgtggaaaccatgttgccatcaaaaatcatgagaaagttagaaccagaagaatttgcagcatatcttgaaccattcaaagagaaaggtgaagttcgtcgtccaacattatcatggcctcgtgaaatcccgttagtaaaaggtggtaaacctgacgttgtacaaattgttaggaattataatgcttatctacgtgcaagtgatgatttaccaaaaatgtttattgaatcggatccaggattcttttccaatgctattgttgaaggcgccaagaagtttcctaatactgaatttgtcaaagtaaaaggtcttcatttttcgcaagaagatgcacctgatgaaatgggaaaatatatcaaatcgttcgttgagcgagttctcaaaaatgaacaataa

SEQ ID NO: 41 - DNA, 2127 bp, Artificial Sequence
  Modified human niacin receptor
atgaatcggcaccatctgcaggatcactttctggaaatagacaagaagaactgctgtgtgttccgagatgacttcattgtcaaggtgttgccgccggtgttggggctggagtttatcttcgggcttctgggcaatggccttgccctgtggattttctgtttccacctcaagtcctggaaatccagccggattttcctgttcaacctggcagtggctgactttctactgatcatctgcctgcccttcctgatggacaactatgtgaggcgttgggactggaagtttggggacatcccttgccggctgatgctcttcatgttggctatgaaccgccagggcagcatcatcttcctcacggtggtggcggtagacaggtatttccgggtggtccatccccaccacgccctgaacaagatctccaatcggacagcagccatcatctcttgccttctgtggggcatcactattggcctgacagtccacctcctgaagaagaagatgccgatccagaatggcggtgcaaatttgtgcagcagcttcagcatctgccataccttccagtggcacgaagccatgttcctcctggagttcttcctgcccctgggcatcatcctgttctgcggtaccagggtctttcaggaggccaaaaggcagctccagaagattgacaaatctgagggccgcttccatgtccagaaccttagccaggtggagcaggatgggcggacggggcatggactccgcagatcttccaagttctgctccagggaaaagaaagcagccaaaaccttggaattcgtggccatcgtctttgtcatctgcttccttcccagcgtggttgtgcggatccgcatcttctggctcctgcacacttcgggcacgcagaattgtgaagtgtaccgctcggtggacctggcgttctttatcactctcagcttcacctacatgaacagcatgctggaccccgtggtgtactacttctccagcccatcctttcccaacttcttctccactttgatcaaccgctgcctccagaggaagatgacaggtgagccagataataaccgcagcacgagcgtcgagctcacaggggaccccaacaaaaccagaggcgctccagaggcgttaatggccaactccggtgagccatggagcccctcttatctgggcccaacctctcctgagaattccatgacttcgaaagtttatgatccagaacaaaggaaacggatgataactggtccgcagtggtgggccagatgtaaacaaatgaatgttcttgattcatttattaattattatgattcagaaaaacatgcagaaaatgctgttatttttttacatggtaacgcggcctcttcttatttatggcgacatgttgtgccacatattgagccagtagcgcggtgtattataccagatcttattggtatgggcaaatcaggcaaatctggtaatggttcttataggttacttgatcattacaaatatcttactgcatggtttgaacttcttaatttaccaaagaagatcaattttgtcggccatgattggggtgcttgtttggcatttcattatagctatgagcatcaagataagatcaaagcaatagttcacgctgaaagtgtagtagatgtgattgaatcatgggatgaatggcctgatattgaagaagatattgcgttgatcaaatctgaagaaggagaaaaaatggttttggagaataacttcttcgtggaaaccatgttgccatcaaaaatcatgagaaagttagaaccagaagaatttgcagcatatcttgaaccattcaaagagaaaggtgaagttcgtcgtccaacattatcatggcctcgtgaaatcccgttagtaaaaggtggtaaacctgacgttgtacaaattgttaggaattataatgcttatctacgtgcaagtgatgatttaccaaaaatgtttattgaatcggatccaggattcttttccaatgctattgttgaaggcgccaagaagtttcctaatactgaatttgtcaaagtaaaaggtcttcatttttcgcaagaagatgcacctgatgaaatgggaaaatatatcaaatcgttcgttgagcgagttctcaaaaatgaacaataa

SEQ ID NO: 42 - DNA, 1950 bp, Artificial Sequence
  Modified human histamine 4 receptor
atgccagatactaatagcacaatcaatttatcactaagcactcgtgttactttagcattttttatgtccttagtagcttttgctataatgctaggaaatgctttggtcattttagcttttgtggtggacaaaaaccttagacatcgaagtagttatttttttcttaacttggccatctctgacttctttgtgggtgtgatctccattcctttgtacatccctcacacgctgttcgaatgggattttggaaaggaaatctgtgtattttggctcactactgactatctgttatgtacagcatctgtatataacattgtcctcatcagctatgatcgatacctgtcagtctcaaatgctgtgtcttatagaactcaacatactggggtcttgaagattgttactctgatggtggccgtttgggtgctggccttcttagtgaatgggccaatgattctagtttcagagtcttggaaggatgaaggtagtgaatgtgaacctggatttttttcggaatggtacatccttgccatcacatcattcttggaattcgtgatcccagtcatcttagtcgcttatttcggtaccagggtctttcaggaggccaaaaggcagctccagaagattgacaaatctgagggccgcttccatgtccagaaccttagccaggtggagcaggatgggcggacggggcatggactccgcagatcttccaagttctgctccagggaaaagaaagcagccaaaaccttggaattcctcttaggggtttttgctgtttgctgggctccatattctctgttcacaattgtcctttcattttattcctcagcaacaggtcctaaatcagtttggtatagaattgcattttggcttcagtggttcaattcctttgtcaatcctcttttgtatccattgtgtcacaagcgctttcaaaaggctttcttgaaaatattttgtataaaaaagcaacctctaccatcacaacacagtcggtcagtatcttctgagaattccatgacttcgaaagtttatgatccagaacaaaggaaacggatgataactggtccgcagtggtgggccagatgtaaacaaatgaatgttcttgattcatttattaattattatgattcagaaaaacatgcagaaaatgctgttatttttttacatggtaacgcggcctcttcttatttatggcgacatgttgtgccacatattgagccagtagcgcggtgtattataccagatcttattggtatgggcaaatcaggcaaatctggtaatggttcttataggttacttgatcattacaaatatcttactgcatggtttgaacttcttaatttaccaaagaagatcaattttgtcggccatgattggggtgcttgtttggcatttcattatagctatgagcatcaagataagatcaaagcaatagttcacgctgaaagtgtagtagatgtgattgaatcatgggatgaatggcctgatattgaagaagatattgcgttgatcaaatctgaagaaggagaaaaaatggttttggagaataacttcttcgtggaaaccatgttgccatcaaaaatcatgagaaagttagaaccagaagaatttgcagcatatcttgaaccattcaaagagaaaggtgaagttcgtcgtccaacattatcatggcctcgtgaaatcccgttagtaaaaggtggtaaacctgacgttgtacaaattgttaggaattataatgcttatctacgtgcaagtgatgatttaccaaaaatgtttattgaatcggatccaggattcttttccaatgctattgttgaaggcgccaagaagtttcctaatactgaatttgtcaaagtaaaaggtcttcatttttcgcaagaagatgcacctgatgaaatgggaaaatatatcaaatcgttcgttgagcgagttctcaaaaatgaacaataa

SEQ ID NO: 43 - DNA, 2118 bp, Artificial Sequence
  Modified human ghrelin receptor
atgtggaacgcgacgcccagcgaagagccggggttcaacctcacactggccgacctggactgggatgcttcccccggcaacgactcgctgggcgacgagctgctgcagctcttccccgcgccgctgctggcgggcgtcacagccacctgcgtggcactcttcgtggtgggtatcgctggcaacctgctcaccatgctggtggtgtcgcgcttccgcgagctgcgcaccaccaccaacctctacctgtccagcatggccttctccgatctgctcatcttcctctgcatgcccctggacctcgttcgcctctggcagtaccggccctggaacttcggcgacctcctctgcaaactcttccaattcgtcagtgagagctgcacctacgccacggtgctcaccatcacagcgctgagcgtcgagcgctacttcgccatctgcttcccactccgggccaaggtggtggtcaccaaggggcgggtgaagctggtcatcttcgtcatctgggccgtggccttctgcagcgccgggcccatcttcgtgctagtcggggtggagcacgagaacggcaccgacccttgggacaccaacgagtgccgccccaccgagtttgcggtgcgctctggactgctcacggtcatggtgtgggtgtccagcatcttcttcttccttcctgtcttctgtctcacggtcctcggtaccagggtctttcaggaggccaaaaggcagctccagaagattgacaaatctgagggccgcttccatgtccagaaccttagccaggtggagcaggatgggcggacggggcatggactccgcagatcttccaagttctgctccagggaaaagaaagcagccaaaaccttggaattcgctgtagtggtgtttgccttcatcctctgctggctccccttccacgtagggcgatatttattttccaaatcctttgagcctggctccttggagattgctcagatcagccagtactgcaacctcgtgtcctttgtcctcttctacctcagtgctgccatcaaccccattctgtacaacatcatgtccaagaagtaccgggtggcagtgttcagacttctgggattcgaacccttctcccagagaaagctctccactctgaaagatgaaagttctcgggcctggacagaatctagtattaatacagagaattccatgacttcgaaagtttatgatccagaacaaaggaaacggatgataactggtccgcagtggtgggccagatgtaaacaaatgaatgttcttgattcatttattaattattatgattcagaaaaacatgcagaaaatgctgttatttttttacatggtaacgcggcctcttcttatttatggcgacatgttgtgccacatattgagccagtagcgcggtgtattataccagatcttattggtatgggcaaatcaggcaaatctggtaatggttcttataggttacttgatcattacaaatatcttactgcatggtttgaacttcttaatttaccaaagaagatcaattttgtcggccatgattggggtgcttgtttggcatttcattatagctatgagcatcaagataagatcaaagcaatagttcacgctgaaagtgtagtagatgtgattgaatcatgggatgaatggcctgatattgaagaagatattgcgttgatcaaatctgaagaaggagaaaaaatggttttggagaataacttcttcgtggaaaccatgttgccatcaaaaatcatgagaaagttagaaccagaagaatttgcagcatatcttgaaccattcaaagagaaaggtgaagttcgtcgtccaacattatcatggcctcgtgaaatcccgttagtaaaaggtggtaaacctgacgttgtacaaattgttaggaattataatgcttatctacgtgcaagtgatgatttaccaaaaatgtttattgaatcggatccaggattcttttccaatgctattgttgaaggcgccaagaagtttcctaatactgaatttgtcaaagtaaaaggtcttcatttttcgcaagaagatgcacctgatgaaatgggaaaatatatcaaatcgttcgttgagcgagttctcaaaaatgaacaataa

SEQ ID NO: 44 - DNA, 2151 bp, Artificial Sequence
  Modified human CXCR3 chemokine receptor
atggtccttgaggtgagtgaccaccaagtgctaaatgacgccgaggttgccgccctcctggagaacttcagctcttcctatgactatggagaaaacgagagtgactcgtgctgtacctccccgccctgcccacaggacttcagcctgaacttcgaccgggccttcctgccagccctctacagcctcctctttctgctggggctgctgggcaacggcgcggtggcagccgtgctgctgagccggcggacagccctgagcagcaccgacaccttcctgctccacctagctgtagcagacacgctgctggtgctgacactgccgctctgggcagtggacgctgccgtccagtgggtctttggctctggcctctgcaaagtggcaggtgccctcttcaacatcaacttctacgcaggagccctcctgctggcctgcatcagctttgaccgctacctgaacatagttcatgccacccagctctaccgccgggggcccccggcccgcgtgaccctcacctgcctggctgtctgggggctctgcctgcttttcgccctcccagacttcatcttcctgtcggcccaccacgacgagcgcctcaacgccacccactgccaatacaacttcccacaggtgggccgcacggctctgcgggtgctgcagctggtggctggctttctgctgcccctgctggtcatggcctactgcggtaccagggtctttcaggaggccaaaaggcagctccagaagattgacaaatctgagggccgcttccatgtccagaaccttagccaggtggagcaggatgggcggacggggcatggactccgcagatcttccaagttctgctccagggaaaagaaagcagccaaaaccttggaattcgtcgtggtggcctttgccctctgctggaccccctatcacctggtggtgctggtggacatcctcatggacctgggcgctttggcccgcaactgtggccgagaaagcagggtagacgtggccaagtcggtcacctcaggcctgggctacatgcactgctgcctcaacccgctgctctatgcctttgtaggggtcaagttccgggagcggatgtggatgctgctcttgcgcctgggctgccccaaccagagagggctccagaggcagccatcgtcttcccgccgggattcatcctggtctgagacctcagaggcctcctactcgggcttggagaattccatgacttcgaaagtttatgatccagaacaaaggaaacggatgataactggtccgcagtggtgggccagatgtaaacaaatgaatgttcttgattcatttattaattattatgattcagaaaaacatgcagaaaatgctgttatttttttacatggtaacgcggcctcttcttatttatggcgacatgttgtgccacatattgagccagtagcgcggtgtattataccagatcttattggtatgggcaaatcaggcaaatctggtaatggttcttataggttacttgatcattacaaatatcttactgcatggtttgaacttcttaatttaccaaagaagatcaattttgtcggccatgattggggtgcttgtttggcatttcattatagctatgagcatcaagataagatcaaagcaatagttcacgctgaaagtgtagtagatgtgattgaatcatgggatgaatggcctgatattgaagaagatattgcgttgatcaaatctgaagaaggagaaaaaatggttttggagaataacttcttcgtggaaaccatgttgccatcaaaaatcatgagaaagttagaaccagaagaatttgcagcatatcttgaaccattcaaagagaaaggtgaagttcgtcgtccaacattatcatggcctcgtgaaatcccgttagtaaaaggtggtaaacctgacgttgtacaaattgttaggaattataatgcttatctacgtgcaagtgatgatttaccaaaaatgtttattgaatcggatccaggattcttttccaatgctattgttgaaggcgccaagaagtttcctaatactgaatttgtcaaagtaaaaggtcttcatttttcgcaagaagatgcacctgatgaaatgggaaaatatatcaaatcgttcgttgagcgagttctcaaaaatgaacaataa

SEQ ID NO: 45 - DNA, 2250 bp, Artificial Sequence
  Modified human motilin receptor
atgggcagcccctggaacggcagcgacggccccgagggggcgcgggagccgccgtggcccgcgctgccgccttgcgacgagcgccgctgctcgccctttcccctgggggcgctggtgccggtgaccgctgtgtgcctgtgcctgttcgtcgtcggggtgagcggcaacgtggtgaccgtgatgctgatcgggcgctaccgggacatgcggaccaccaccaacttgtacctgggcagcatggccgtgtccgacctactcatcctgctcgggctgccgttcgacctgtaccgcctctggcgctcgcggccctgggtgttcgggccgctgctctgccgcctgtccctctacgtgggcgagggctgcacctacgccacgctgctgcacatgaccgcgctcagcgtcgagcgctacctggccatctgccgcccgctccgcgcccgcgtcttggtcacccggcgccgcgtccgcgcgctcatcgctgtgctctgggccgtggcgctgctctctgccggtcccttcttgttcctggtgggcgtcgagcaggaccccggcatctccgtagtcccgggcctcaatggcaccgcgcggatcgcctcctcgcctctcgcctcgtcgccgcctctctggctctcgcgggcgccaccgccgtccccgccgtcggggcccgagaccgcggaggccgcggcgctgttcagccgcgaatgccggccgagccccgcgcagctgggcgcgctgcgtgtcatgctgtgggtcaccaccgcctacttcttcctgccctttctgtgcctcagcatcctcggtaccagggtctttcaggaggccaaaaggcagctccagaagattgacaaatctgagggccgcttccatgtccagaaccttagccaggtggagcaggatgggcggacggggcatggactccgcagatcttccaagttctgctccagggaaaagaaagcagccaaaaccttggaattcgtggttctggcatttataatttgctggttgcccttccacgttggcagaatcatttacataaacacggaagattcgcggatgatgtacttctctcagtactttaacatcgtcgctctgcaacttttctatctgagcgcatctatcaacccaatcctctacaacctcatttcaaagaagtacagagcggcggcctttaaactgctgctcgcaaggaagtccaggccgagaggcttccacagaagcagggacactgcgggggaagttgcaggggacactggaggagacacggtgggctacaccgagacaagcgctaacgtgaagacgatgggagagaattccatgacttcgaaagtttatgatccagaacaaaggaaacggatgataactggtccgcagtggtgggccagatgtaaacaaatgaatgttcttgattcatttattaattattatgattcagaaaaacatgcagaaaatgctgttatttttttacatggtaacgcggcctcttcttatttatggcgacatgttgtgccacatattgagccagtagcgcggtgtattataccagatcttattggtatgggcaaatcaggcaaatctggtaatggttcttataggttacttgatcattacaaatatcttactgcatggtttgaacttcttaatttaccaaagaagatcaattttgtcggccatgattggggtgcttgtttggcatttcattatagctatgagcatcaagataagatcaaagcaatagttcacgctgaaagtgtagtagatgtgattgaatcatgggatgaatggcctgatattgaagaagatattgcgttgatcaaatctgaagaaggagaaaaaatggttttggagaataacttcttcgtggaaaccatgttgccatcaaaaatcatgagaaagttagaaccagaagaatttgcagcatatcttgaaccattcaaagagaaaggtgaagttcgtcgtccaacattatcatggcctcgtgaaatcccgttagtaaaaggtggtaaacctgacgttgtacaaattgttaggaattataatgcttatctacgtgcaagtgatgatttaccaaaaatgtttattgaatcggatccaggattcttttccaatgctattgttgaaggcgccaagaagtttcctaatactgaatttgtcaaagtaaaaggtcttcatttttcgcaagaagatgcacctgatgaaatgggaaaatatatcaaatcgttcgttgagcgagttctcaaaaatgaacaataa

SEQ ID NO: 46 - DNA, 2313 bp, Artificial Sequence
  Modified human 5-hydroxytryptamine (serotonin) receptor 2A
atggatattctttgtgaagaaaatacttctttgagctcaactacgaactccctaatgcaattaaatgatgacaccaggctctacagtaatgactttaactccggagaagctaacacttctgatgcatttaactggacagtcgactctgaaaatcgaaccaacctttcctgtgaagggtgcctctcaccgtcgtgtctctccttacttcatctccaggaaaaaaactggtctgctttactgacagccgtagtgattattctaactattgctggaaacatactcgtcatcatggcagtgtccctagagaaaaagctgcagaatgccaccaactatttcctgatgtcacttgccatagctgatatgctgctgggtttccttgtcatgcccgtgtccatgttaaccatcctgtatgggtaccggtggcctctgccgagcaagctttgtgcagtctggatttacctggacgtgctcttctccacggcctccatcatgcacctctgcgccatctcgctggaccgctacgtcgccatccagaatcccatccaccacagccgcttcaactccagaactaaggcatttctgaaaatcattgctgtttggaccatatcagtaggtatatccatgccaataccagtctttgggctacaggacgattcgaaggtctttaaggaggggagttgcttactcgccgatgataactttgtcctgatcggctcttttgtgtcatttttcattcccttaaccatcatggtgatcaccggtaccagggtctttcaggaggccaaaaggcagctccagaagattgacaaatctgagggccgcttccatgtccagaaccttagccaggtggagcaggatgggcggacggggcatggactccgcagatcttccaagttctgctccagggaaaagaaagcagccaaaaccttggaattcgtcttcttcctgtttgtggtgatgtggtgccctttcttcatcacaaacatcatggccgtcatctgcaaagagtcctgcaatgaggatgtcattggggccctgctcaatgtgtttgtttggatcggttatctctcttcagcagtcaacccactagtctacacactgttcaacaagacctataggtcagccttttcacggtatattcagtgtcagtacaaggaaaacaaaaaaccattgcagttaattttagtgaacacaataccggctttggcctacaagtctagccaacttcaaatgggacaaaaaaagaattcaaagcaagatgccaagacaacagataatgactgctcaatggttgctctaggaaagcagcattctgaagaggcttctaaagacaatagcgacggagtgaatgaaaaggtgagctgtgtggagaattccatgacttcgaaagtttatgatccagaacaaaggaaacggatgataactggtccgcagtggtgggccagatgtaaacaaatgaatgttcttgattcatttattaattattatgattcagaaaaacatgcagaaaatgctgttatttttttacatggtaacgcggcctcttcttatttatggcgacatgttgtgccacatattgagccagtagcgcggtgtattataccagatcttattggtatgggcaaatcaggcaaatctggtaatggttcttataggttacttgatcattacaaatatcttactgcatggtttgaacttcttaatttaccaaagaagatcaattttgtcggccatgattggggtgcttgtttggcatttcattatagctatgagcatcaagataagatcaaagcaatagttcacgctgaaagtgtagtagatgtgattgaatcatgggatgaatggcctgatattgaagaagatattgcgttgatcaaatctgaagaaggagaaaaaatggttttggagaataacttcttcgtggaaaccatgttgccatcaaaaatcatgagaaagttagaaccagaagaatttgcagcatatcttgaaccattcaaagagaaaggtgaagttcgtcgtccaacattatcatggcctcgtgaaatcccgttagtaaaaggtggtaaacctgacgttgtacaaattgttaggaattataatgcttatctacgtgcaagtgatgatttaccaaaaatgtttattgaatcggatccaggattcttttccaatgctattgttgaaggcgccaagaagtttcctaatactgaatttgtcaaagtaaaaggtcttcatttttcgcaagaagatgcacctgatgaaatgggaaaatatatcaaatcgttcgttgagcgagttctcaaaaatgaacaataa

SEQ ID NO: 47 - DNA, 2289 bp, Artificial Sequence
  Modified human 5-hydroxytryptamine (serotonin) receptor 2B
atggctctctcttacagagtgtctgaacttcaaagcacaattcctgagcacattttgcagagcacctttgttcacgttatctcttctaactggtctggattacagacagaatcaataccagaggaaatgaaacagattgttgaggaacagggaaataaactgcactgggcagctcttctgatactcatggtgataatacccacaattggtggaaatacccttgttattctggctgtttcactggagaagaagctgcagtatgctactaattactttctaatgtccttggcggtggctgatttgctggttggattgtttgtgatgccaattgccctcttgacaataatgtttgaggctatgtggcccctcccacttgttctatgtcctgcctggttatttcttgacgttctcttttcaaccgcatccatcatgcatctctgtgccatttcagtggatcgttacatagccatcaaaaagccaatccaggccaatcaatataactcacgggctacagcattcatcaagattacagtggtgtggttaatttcaataggcattgccattccagtccctattaaagggatagagactgatgtggacaacccaaacaatatcacttgtgtgctgacaaaggaacgttttggcgatttcatgctctttggctcactggctgccttcttcacacctcttgcaattatgattgtcaccggtaccagggtctttcaggaggccaaaaggcagctccagaagattgacaaatctgagggccgcttccatgtccagaaccttagccaggtggagcaggatgggcggacggggcatggactccgcagatcttccaagttctgctccagggaaaagaaagcagccaaaaccttggaattcgtgtttttcctctttttgcttatgtggtgtcccttctttattacaaatataactttagttttatgtgattcctgtaaccaaactactctccaaatgctcctggagatatttgtgtggataggctatgtttcctcaggagtgaatcctttggtctacaccctcttcaataagacatttcgggatgcatttggccgatatatcacctgcaattaccgggccacaaagtcagtaaaaactctcagaaaacgctccagtaagatctacttccggaatccaatggcagagaactctaagtttttcaagaaacatggaattcgaaatgggattaaccctgccatgtaccagagtccaatgaggctccgaagttcaaccattcagtcttcatcaatcattctactagatacgcttctcctcactgaaaatgaaggtgacaaaactgaagagcaagttagttatgtagagaattccatgacttcgaaagtttatgatccagaacaaaggaaacggatgataactggtccgcagtggtgggccagatgtaaacaaatgaatgttcttgattcatttattaattattatgattcagaaaaacatgcagaaaatgctgttatttttttacatggtaacgcggcctcttcttatttatggcgacatgttgtgccacatattgagccagtagcgcggtgtattataccagatcttattggtatgggcaaatcaggcaaatctggtaatggttcttataggttacttgatcattacaaatatcttactgcatggtttgaacttcttaatttaccaaagaagatcaattttgtcggccatgattggggtgcttgtttggcatttcattatagctatgagcatcaagataagatcaaagcaatagttcacgctgaaagtgtagtagatgtgattgaatcatgggatgaatggcctgatattgaagaagatattgcgttgatcaaatctgaagaaggagaaaaaatggttttggagaataacttcttcgtggaaaccatgttgccatcaaaaatcatgagaaagttagaaccagaagaatttgcagcatatcttgaaccattcaaagagaaaggtgaagttcgtcgtccaacattatcatggcctcgtgaaatcccgttagtaaaaggtggtaaacctgacgttgtacaaattgttaggaattataatgcttatctacgtgcaagtgatgatttaccaaaaatgtttattgaatcggatccaggattcttttccaatgctattgttgaaggcgccaagaagtttcctaatactgaatttgtcaaagtaaaaggtcttcatttttcgcaagaagatgcacctgatgaaatgggaaaatatatcaaatcgttcgttgagcgagttctcaaaaatgaacaataa

SEQ ID NO: 48 - DNA, 2250 bp, Artificial Sequence
  Modified human 5-hydroxytrptamine (serotonin) receptor 2C
atggtgaacctgaggaatgcggtgcattcattccttgtgcacctaattggcctattggtttggcaatgtgatatttctgtgagcccagtagcagctatagtaactgacattttcaatacctccgatggtggacgcttcaaattcccagacggggtacaaaactggccagcactttcaatcgtcatcataataatcatgacaataggtggcaacatccttgtgatcatggcagtaagcatggaaaagaaactgcacaatgccaccaattacttcttaatgtccctagccattgctgatatgctagtgggactacttgtcatgcccctgtctctcctggcaatcctttatgattatgtctggccactacctagatatttgtgccccgtctggatttctttagatgttttattttcaacagcgtccatcatgcacctctgcgctatatcgctggatcggtatgtagcaatacgtaatcctattgagcatagccgtttcaattcgcggactaaggccatcatgaagattgctattgtttgggcaatttctataggtgtatcagttcctatccctgtgattggactgagggacgaagaaaaggtgttcgtgaacaacacgacgtgcgtgctcaacgacccaaatttcgttcttattgggtccttcgtagctttcttcataccgctgacgattatggtgattacgggtaccagggtctttcaggaggccaaaaggcagctccagaagattgacaaatctgagggccgcttccatgtccagaaccttagccaggtggagcaggatgggcggacggggcatggactccgcagatcttccaagttctgctccagggaaaagaaagcagccaaaaccttggaattcgttttctttgtgtttctgatcatgtggtgcccatttttcattaccaatattctgtctgttctttgtgagaagtcctgtaaccaaaagctcatggaaaagcttctgaatgtgtttgtttggattggctatgtttgttcaggaatcaatcctctggtgtatactctgttcaacaaaatttaccgaagggcattctccaactatttgcgttgcaattataaggtagagaaaaagcctcctgtcaggcagattccaagagttgccgccactgctttgtctgggagggagcttaatgttaacatttatcggcataccaatgaaccggtgatcgagaaagccagtgacaatgagcccggtatagagatgcaagttgagaatttagagttaccagtaaatccctccagtgtggttagcgaaaggattagcagtgtggagaattccatgacttcgaaagtttatgatccagaacaaaggaaacggatgataactggtccgcagtggtgggccagatgtaaacaaatgaatgttcttgattcatttattaattattatgattcagaaaaacatgcagaaaatgctgttatttttttacatggtaacgcggcctcttcttatttatggcgacatgttgtgccacatattgagccagtagcgcggtgtattataccagatcttattggtatgggcaaatcaggcaaatctggtaatggttcttataggttacttgatcattacaaatatcttactgcatggtttgaacttcttaatttaccaaagaagatcaattttgtcggccatgattggggtgcttgtttggcatttcattatagctatgagcatcaagataagatcaaagcaatagttcacgctgaaagtgtagtagatgtgattgaatcatgggatgaatggcctgatattgaagaagatattgcgttgatcaaatctgaagaaggagaaaaaatggttttggagaataacttcttcgtggaaaccatgttgccatcaaaaatcatgagaaagttagaaccagaagaatttgcagcatatcttgaaccattcaaagagaaaggtgaagttcgtcgtccaacattatcatggcctcgtgaaatcccgttagtaaaaggtggtaaacctgacgttgtacaaattgttaggaattataatgcttatctacgtgcaagtgatgatttaccaaaaatgtttattgaatcggatccaggattcttttccaatgctattgttgaaggcgccaagaagtttcctaatactgaatttgtcaaagtaaaaggtcttcatttttcgcaagaagatgcacctgatgaaatgggaaaatatatcaaatcgttcgttgagcgagttctcaaaaatgaacaataa

SEQ ID NO: 49 - DNA, 1944 bp, Artificial Sequence
  Modified human dopamine receptor D3
atggcatctctgagtcagctgagtagccacctgaactacacctgtggggcagagaactccacaggtgccagccaggcccgcccacatgcctactatgccctctcctactgcgcgctcatcctggccatcgtcttcggcaatggcctggtgtgcatggctgtgctgaaggagcgggccctgcagactaccaccaactacttagtagtgagcctggctgtggcagacttgctggtggccaccttggtgatgccctgggtggtatacctggaggtgacaggtggagtctggaatttcagccgcatttgctgtgatgtttttgtcaccctggatgtcatgatgtgtacagccagcatccttaatctctgtgccatcagcatagacaggtacactgcagtggtcatgcccgttcactaccagcatggcacgggacagagctcctgtcggcgcgtggccctcatgatcacggccgtctgggtactggcctttgctgtgtcctgccctcttctgtttggctttaataccacaggggaccccactgtctgctccatctccaaccctgattttgtcatctactcttcagtggtgtccttctacctgccctttggagtgactgtccttgtcggtaccagggtctttcaggaggccaaaaggcagctccagaagattgacaaatctgagggccgcttccatgtccagaaccttagccaggtggagcaggatgggcggacggggcatggactccgcagatcttccaagttctgctccagggaaaagaaagcagccaaaaccttggaattcgtgcttggggccttcattgtctgctggctgcccttcttcttgacccatgttctcaatacccactgccagacatgccacgtgtccccagagctttacagtgccacgacatggctgggctacgtgaatagcgccctcaaccctgtgatctataccaccttcaatatcgagttccggaaagccttcctcaagatcctgtcttgcgagaattccatgacttcgaaagtttatgatccagaacaaaggaaacggatgataactggtccgcagtggtgggccagatgtaaacaaatgaatgttcttgattcatttattaattattatgattcagaaaaacatgcagaaaatgctgttatttttttacatggtaacgcggcctcttcttatttatggcgacatgttgtgccacatattgagccagtagcgcggtgtattataccagatcttattggtatgggcaaatcaggcaaatctggtaatggttcttataggttacttgatcattacaaatatcttactgcatggtttgaacttcttaatttaccaaagaagatcaattttgtcggccatgattggggtgcttgtttggcatttcattatagctatgagcatcaagataagatcaaagcaatagttcacgctgaaagtgtagtagatgtgattgaatcatgggatgaatggcctgatattgaagaagatattgcgttgatcaaatctgaagaaggagaaaaaatggttttggagaataacttcttcgtggaaaccatgttgccatcaaaaatcatgagaaagttagaaccagaagaatttgcagcatatcttgaaccattcaaagagaaaggtgaagttcgtcgtccaacattatcatggcctcgtgaaatcccgttagtaaaaggtggtaaacctgacgttgtacaaattgttaggaattataatgcttatctacgtgcaagtgatgatttaccaaaaatgtttattgaatcggatccaggattcttttccaatgctattgttgaaggcgccaagaagtttcctaatactgaatttgtcaaagtaaaaggtcttcatttttcgcaagaagatgcacctgatgaaatgggaaaatatatcaaatcgttcgttgagcgagttctcaaaaatgaacaataa

SEQ ID NO: 50 - DNA, 1968 bp, Artificial Sequence
  Modified human dopamine receptor D4
atggggaaccgcagcaccgcggacgcggacgggctgctggctgggcgcgggccggccgcgggggcatctgcgggggcatctgcggggctggctgggcagggcgcggcggcgctggtggggggcgtgctgctcatcggcgcggtgctcgcggggaactcgctcgtgtgcgtgagcgtggccaccgagcgcgccctgcagacgcccaccaactccttcatcgtgagcctggcggccgccgacctcctcctcgctctcctggtgctgccgctcttcgtctactccgaggtccagggtggcgcgtggctgctgagcccccgcctgtgcgacgccctcatggccatggacgtcatgctgtgcaccgcctccatcttcaacctgtgcgccatcagcgtggacaggttcgtggccgtggccgtgccgctgcgctacaaccggcagggtgggagccgccggcagctgctgctcatcggcgccacgtggctgctgtccgcggcggtggcggcgcccgtactgtgcggcctcaacgacgtgcgcggccgcgaccccgccgtgtgccgcctggaggaccgcgactacgtggtctactcgtccgtgtgctccttcttcctaccctgcccgctcatgctgctgctctactggggtaccagggtctttcaggaggccaaaaggcagctccagaagattgacaaatctgagggccgcttccatgtccagaaccttagccaggtggagcaggatgggcggacggggcatggactccgcagatcttccaagttctgctccagggaaaagaaagcagccaaaaccttggaattcgtggtcggggccttcctgctgtgctggacgcccttcttcgtggtgcacatcacgcaggcgctgtgtcctgcctgctccgtgcccccgcggctggtcagcgccgtcacctggctgggctacgtcaacagcgccctcaaccccgtcatctacactgtcttcaacgccgagttccgcaacgtcttccgcaaggccctgcgtgcctgctgcgagaattccatgacttcgaaagtttatgatccagaacaaaggaaacggatgataactggtccgcagtggtgggccagatgtaaacaaatgaatgttcttgattcatttattaattattatgattcagaaaaacatgcagaaaatgctgttatttttttacatggtaacgcggcctcttcttatttatggcgacatgttgtgccacatattgagccagtagcgcggtgtattataccagatcttattggtatgggcaaatcaggcaaatctggtaatggttcttataggttacttgatcattacaaatatcttactgcatggtttgaacttcttaatttaccaaagaagatcaattttgtcggccatgattggggtgcttgtttggcatttcattatagctatgagcatcaagataagatcaaagcaatagttcacgctgaaagtgtagtagatgtgattgaatcatgggatgaatggcctgatattgaagaagatattgcgttgatcaaatctgaagaaggagaaaaaatggttttggagaataacttcttcgtggaaaccatgttgccatcaaaaatcatgagaaagttagaaccagaagaatttgcagcatatcttgaaccattcaaagagaaaggtgaagttcgtcgtccaacattatcatggcctcgtgaaatcccgttagtaaaaggtggtaaacctgacgttgtacaaattgttaggaattataatgcttatctacgtgcaagtgatgatttaccaaaaatgtttattgaatcggatccaggattcttttccaatgctattgttgaaggcgccaagaagtttcctaatactgaatttgtcaaagtaaaaggtcttcatttttcgcaagaagatgcacctgatgaaatgggaaaatatatcaaatcgttcgttgagcgagttctcaaaaatgaacaataa

SEQ ID NO: 51 - DNA, 2271 bp, Artificial Sequence
  Modified human dopamine receptor D1
atgaggactctgaacacctctgccatggacgggactgggctggtggtggagagggacttctctgttcgtatcctcactgcctgtttcctgtcgctgctcatcctgtccacgctcctggggaacacgctggtctgtgctgccgttatcaggttccgacacctgcggtccaaggtgaccaacttctttgtcatctccttggctgtgtcagatctcttggtggccgtcctggtcatgccctggaaggcagtggctgagattgctggcttctggccctttgggtccttctgtaacatctgggtggcctttgacatcatgtgctccactgcatccatcctcaacctctgtgtgatcagcgtggacaggtattgggctatctccagccctttccggtatgagagaaagatgacccccaaggcagccttcatcctgatcagtgtggcatggaccttgtctgtactcatctccttcatcccagtgcagctcagctggcacaaggcaaaacccacaagcccctctgatggaaatgccacttccctggctgagaccatagacaactgtgactccagcctcagcaggacatatgccatctcatcctctgtaataagcttttacatccctgtggccatcatgattgtcaccggtaccagggtctttcaggaggccaaaaggcagctccagaagattgacaaatctgagggccgcttccatgtccagaaccttagccaggtggagcaggatgggcggacggggcatggactccgcagatcttccaagttctgctccagggaaaagaaagcagccaaaaccttggaattcatcatgggtgtgtttgtgtgctgttggctacctttcttcatcttgaactgcattttgcccttctgtgggtctggggagacgcagcccttctgcattgattccaacacctttgacgtgtttgtgtggtttgggtgggctaattcatccttgaaccccatcatttatgcctttaatgctgattttcggaaggcattttcaaccctcttaggatgctacagactttgccctgcgacgaataatgccatagagacggtgagtatcaataacaatggggccgcgatgttttccagccatcatgagccacgaggctccatctccaaggagtgcaatctggtttacctgatcccacatgctgtgggctcctctgaggacctgaaaaaggaggaggcagctggcatcgccagacccttggagaagctgtccccagccctatcggtcatattggactatgacactgacgtctctctggagaagatccaacccatgacacaaaacggtcagcacccaaccgagaattccatgacttcgaaagtttatgatccagaacaaaggaaacggatgataactggtccgcagtggtgggccagatgtaaacaaatgaatgttcttgattcatttattaattattatgattcagaaaaacatgcagaaaatgctgttatttttttacatggtaacgcggcctcttcttatttatggcgacatgttgtgccacatattgagccagtagcgcggtgtattataccagatcttattggtatgggcaaatcaggcaaatctggtaatggttcttataggttacttgatcattacaaatatcttactgcatggtttgaacttcttaatttaccaaagaagatcaattttgtcggccatgattggggtgcttgtttggcatttcattatagctatgagcatcaagataagatcaaagcaatagttcacgctgaaagtgtagtagatgtgattgaatcatgggatgaatggcctgatattgaagaagatattgcgttgatcaaatctgaagaaggagaaaaaatggttttggagaataacttcttcgtggaaaccatgttgccatcaaaaatcatgagaaagttagaaccagaagaatttgcagcatatcttgaaccattcaaagagaaaggtgaagttcgtcgtccaacattatcatggcctcgtgaaatcccgttagtaaaaggtggtaaacctgacgttgtacaaattgttaggaattataatgcttatctacgtgcaagtgatgatttaccaaaaatgtttattgaatcggatccaggattcttttccaatgctattgttgaaggcgccaagaagtttcctaatactgaatttgtcaaagtaaaaggtcttcatttttcgcaagaagatgcacctgatgaaatgggaaaatatatcaaatcgttcgttgagcgagttctcaaaaatgaacaataa

SEQ ID NO: 52 - DNA, 2088 bp, Artificial Sequence
  Modified human histamine receptor H2
atggcacccaatggcacagcctcttccttttgcctggactctaccgcatgcaagatcaccatcaccgtggtccttgcggtcctcatcctcatcaccgttgctggcaatgtggtcgtctgtctggccgtgggcttgaaccgccggctccgcaacctgaccaattgtttcatcgtgtccttggctatcactgacctgctcctcggcctcctggtgctgcccttctctgccatctaccagctgtcctgcaagtggagctttggcaaggtcttctgcaatatctacaccagcctggatgtgatgctctgcacagcctccattcttaacctcttcatgatcagcctcgaccggtactgcgctgtcatggacccactgcggtaccctgtgctggtcaccccagttcgggtcgccatctctctggtcttaatttgggtcatctccattaccctgtcctttctgtctatccacctggggtggaacagcaggaacgagaccagcaagggcaatcataccacctctaagtgcaaagtccaggtcaatgaagtgtacgggctggtggatgggctggtcaccttctacctcccgctactgatcatgtgcatcaccggtaccagggtctttcaggaggccaaaaggcagctccagaagattgacaaatctgagggccgcttccatgtccagaaccttagccaggtggagcaggatgggcggacggggcatggactccgcagatcttccaagttctgctccagggaaaagaaagcagccaaaaccttggaattcgtcatgggggccttcatcatctgctggtttccctacttcaccgcgtttgtgtaccgtgggctgagaggggatgatgccatcaatgaggtgttagaagccatcgttctgtggctgggctatgccaactcagccctgaaccccatcctgtatgctgcgctgaacagagacttccgcaccgggtaccaacagctcttctgctgcaggctggccaaccgcaactcccacaaaacttctctgaggtccaacgcctctcagctgtccaggacccaaagccgagaacccaggcaacaggaagagaaacccctgaagctccaggtgtggagtgggacagaagtcacggccccccagggagccacagacagggagaattccatgacttcgaaagtttatgatccagaacaaaggaaacggatgataactggtccgcagtggtgggccagatgtaaacaaatgaatgttcttgattcatttattaattattatgattcagaaaaacatgcagaaaatgctgttatttttttacatggtaacgcggcctcttcttatttatggcgacatgttgtgccacatattgagccagtagcgcggtgtattataccagatcttattggtatgggcaaatcaggcaaatctggtaatggttcttataggttacttgatcattacaaatatcttactgcatggtttgaacttcttaatttaccaaagaagatcaattttgtcggccatgattggggtgcttgtttggcatttcattatagctatgagcatcaagataagatcaaagcaatagttcacgctgaaagtgtagtagatgtgattgaatcatgggatgaatggcctgatattgaagaagatattgcgttgatcaaatctgaagaaggagaaaaaatggttttggagaataacttcttcgtggaaaccatgttgccatcaaaaatcatgagaaagttagaaccagaagaatttgcagcatatcttgaaccattcaaagagaaaggtgaagttcgtcgtccaacattatcatggcctcgtgaaatcccgttagtaaaaggtggtaaacctgacgttgtacaaattgttaggaattataatgcttatctacgtgcaagtgatgatttaccaaaaatgtttattgaatcggatccaggattcttttccaatgctattgttgaaggcgccaagaagtttcctaatactgaatttgtcaaagtaaaaggtcttcatttttcgcaagaagatgcacctgatgaaatgggaaaatatatcaaatcgttcgttgagcgagttctcaaaaatgaacaataa

SEQ ID NO: 53 - DNA, 2022 bp, Artificial Sequence
  Modified human histamine receptor H3
atggagcgcgcgccgcccgacgggccgctgaacgcttcgggggcgctggcgggcgatgcggcggcggcgggcggggcgcgcggcttctcggcagcctggaccgcggtgctggccgcgctcatggcgctgctcatcgtggccacggtgctgggcaacgcgctggtcatgctcgccttcgtggccgactcgagcctccgcacccagaacaacttcttcctgctcaacctcgccatctccgacttcctcgtcggcgccttctgcatcccactgtatgtaccctacgtgctgacaggccgctggaccttcggccggggcctctgcaagctgtggctggtagtggactacctgctgtgcacctcctctgccttcaacatcgtgctcatcagctacgaccgcttcctgtcggtcacccgagcggtctcataccgggcccagcagggtgacacgcggcgggcagtgcggaagatgctgctggtgtgggtgctggccttcctgctgtacggaccagccatcctgagctgggagtacctgtccgggggcagctccatccccgagggccactgctatgccgagttcttctacaactggtacttcctcatcacggcttccaccctggagttctttacgcccttcctcagcgtcaccttctttggtaccagggtctttcaggaggccaaaaggcagctccagaagattgacaaatctgagggccgcttccatgtccagaaccttagccaggtggagcaggatgggcggacggggcatggactccgcagatcttccaagttctgctccagggaaaagaaagcagccaaaaccttggaattcatcgtgagcatctttgggctctgctgggccccatacacgctgctgatgatcatccgggccgcctgccatggccactgcgtccctgactactggtacgaaacctccttctggctcctgtgggccaactcggctgtcaaccctgtcctctaccctctgtgccaccacagcttccgccgggccttcaccaagctgctctgcccccagaagctcaaaatccagccccacagctccctggagcactgctggaaggagaattccatgacttcgaaagtttatgatccagaacaaaggaaacggatgataactggtccgcagtggtgggccagatgtaaacaaatgaatgttcttgattcatttattaattattatgattcagaaaaacatgcagaaaatgctgttatttttttacatggtaacgcggcctcttcttatttatggcgacatgttgtgccacatattgagccagtagcgcggtgtattataccagatcttattggtatgggcaaatcaggcaaatctggtaatggttcttataggttacttgatcattacaaatatcttactgcatggtttgaacttcttaatttaccaaagaagatcaattttgtcggccatgattggggtgcttgtttggcatttcattatagctatgagcatcaagataagatcaaagcaatagttcacgctgaaagtgtagtagatgtgattgaatcatgggatgaatggcctgatattgaagaagatattgcgttgatcaaatctgaagaaggagaaaaaatggttttggagaataacttcttcgtggaaaccatgttgccatcaaaaatcatgagaaagttagaaccagaagaatttgcagcatatcttgaaccattcaaagagaaaggtgaagttcgtcgtccaacattatcatggcctcgtgaaatcccgttagtaaaaggtggtaaacctgacgttgtacaaattgttaggaattataatgcttatctacgtgcaagtgatgatttaccaaaaatgtttattgaatcggatccaggattcttttccaatgctattgttgaaggcgccaagaagtttcctaatactgaatttgtcaaagtaaaaggtcttcatttttcgcaagaagatgcacctgatgaaatgggaaaatatatcaaatcgttcgttgagcgagttctcaaaaatgaacaataa

SEQ ID NO: 54 - DNA, 1983 bp, Artificial Sequence
  Modified pig adrenergic receptor, alpha-2A
atgggctccctgcagccggaagcgggcaacgcgagctggaatgggacagaggcgccggggggcggcgcccgggccaccccctactccctgcaggtgacactgacgctggtgtgcctggccggcctgctcatgctgttcaccgtgttcggcaacgtgcttgtcatcattgccgtgttcacaagccgcgcgctcaaggcgccccagaacctcttcctggtgtctctggcctcggctgacatcctagtggccacgcttgtcatccctttctcgctggccaacgaggtcatgggctactggtacttcggcaaggcgtggtgtgagatctacctggcgctcgacgtgctcttctgcacgtcgtccatcgtgcacctgtgtgccatcagcttggatcgttactggtccatcacccaggccatagagtacaacctgaagcgcacgccacgccgcatcaaagcaatcatcgtcaccgtgtgggtcatctcggccgtcatctccttcccgccgctcatctccatcgagaagaaggcaggcggcggtggccagcagccggccgaaccgcgctgcgagatcaacgaccagaagtggtacgtcatctcgtcttgcatcggctccttcttcgcgccctgcctcatcatgatcctggtcggtaccagggtctttcaggaggccaaaaggcagctccagaagattgacaaatctgagggccgcttccatgtccagaaccttagccaggtggagcaggatgggcggacggggcatggactccgcagatcttccaagttctgctccagggaaaagaaagcagccaaaaccttggaattcgtcataggcgtgttcgtggtctgttggttccccttcttcttcacctatacgctcacggccgtaggctgctcggtgccgcccactctcttcaagttcttcttctggttcggctactgcaacagctcgctgaatccggttatctacaccatcttcaatcacgacttccgccgcgccttcaagaagatcctctgccgtggggacaggaaacggatcgtggagaattccatgacttcgaaagtttatgatccagaacaaaggaaacggatgataactggtccgcagtggtgggccagatgtaaacaaatgaatgttcttgattcatttattaattattatgattcagaaaaacatgcagaaaatgctgttatttttttacatggtaacgcggcctcttcttatttatggcgacatgttgtgccacatattgagccagtagcgcggtgtattataccagatcttattggtatgggcaaatcaggcaaatctggtaatggttcttataggttacttgatcattacaaatatcttactgcatggtttgaacttcttaatttaccaaagaagatcaattttgtcggccatgattggggtgcttgtttggcatttcattatagctatgagcatcaagataagatcaaagcaatagttcacgctgaaagtgtagtagatgtgattgaatcatgggatgaatggcctgatattgaagaagatattgcgttgatcaaatctgaagaaggagaaaaaatggttttggagaataacttcttcgtggaaaccatgttgccatcaaaaatcatgagaaagttagaaccagaagaatttgcagcatatcttgaaccattcaaagagaaaggtgaagttcgtcgtccaacattatcatggcctcgtgaaatcccgttagtaaaaggtggtaaacctgacgttgtacaaattgttaggaattataatgcttatctacgtgcaagtgatgatttaccaaaaatgtttattgaatcggatccaggattcttttccaatgctattgttgaaggcgccaagaagtttcctaatactgaatttgtcaaagtaaaaggtcttcatttttcgcaagaagatgcacctgatgaaatgggaaaatatatcaaatcgttcgttgagcgagttctcaaaaatgaacaataa

SEQ ID NO: 55 - DNA, 2073 bp, Artificial Sequence
  Modified human galanin receptor 1
atggagctggcggtcgggaacctcagcgagggcaacgcgagctgtccggagccccccgccccggagcccgggccgctgttcggcatcggcgtggagaacttcgtcacgctggtggtgttcggcctgatcttcgcgctgggcgtgctgggcaacagcctagtgatcaccgtgctggcgcgcagcaagccgggcaagccgcggagcaccaccaacctgttcatcctcaacctgagcatcgccgacctggcctacctgctcttctgcatccccttccaggccaccgtgtacgcgctgcccacctgggtgctgggcgccttcatctgcaagttcatccactacttcttcaccgtgtccatgctggtgagcatcttcaccctggccgcgatgtccgtggaccgctacgtggccatcgtgcactcgcggcgctcctcctccctcagggtgtcccgcaacgcgctgctgggcgtgggctgcatctgggcgctgtccattgccatggcctcgcccgtggcctaccaccagggcctcttccacccgcgcgccagcaaccagaccttctgctgggagcagtggcccgaccctcgccacaagaaggcctacgtggtgtgcaccttcgtcttcggctacctgctgccgctcctgctcatctgcttctgcggtaccagggtctttcaggaggccaaaaggcagctccagaagattgacaaatctgagggccgcttccatgtccagaaccttagccaggtggagcaggatgggcggacggggcatggactccgcagatcttccaagttctgctccagggaaaagaaagcagccaaaaccttggaattcgtggttgtggtgtttggaatctcctggctgccgcaccacatcatccatctctgggctgagtttggagttttcccgctgacgccggcttccttcctcttcagaatcaccgcccactgcctggcgtacagcaattcctccgtgaatcctatcatttatgcatttctctctgaaaatttcaggaaggcctataaacaagtgttcaagtgtcacattcgcaaagattcacacctgagtgatactaaagaaaataaaagtcgaatagacaccccaccatcaaccaattgtactcatgtggagaattccatgacttcgaaagtttatgatccagaacaaaggaaacggatgataactggtccgcagtggtgggccagatgtaaacaaatgaatgttcttgattcatttattaattattatgattcagaaaaacatgcagaaaatgctgttatttttttacatggtaacgcggcctcttcttatttatggcgacatgttgtgccacatattgagccagtagcgcggtgtattataccagatcttattggtatgggcaaatcaggcaaatctggtaatggttcttataggttacttgatcattacaaatatcttactgcatggtttgaacttcttaatttaccaaagaagatcaattttgtcggccatgattggggtgcttgtttggcatttcattatagctatgagcatcaagataagatcaaagcaatagttcacgctgaaagtgtagtagatgtgattgaatcatgggatgaatggcctgatattgaagaagatattgcgttgatcaaatctgaagaaggagaaaaaatggttttggagaataacttcttcgtggaaaccatgttgccatcaaaaatcatgagaaagttagaaccagaagaatttgcagcatatcttgaaccattcaaagagaaaggtgaagttcgtcgtccaacattatcatggcctcgtgaaatcccgttagtaaaaggtggtaaacctgacgttgtacaaattgttaggaattataatgcttatctacgtgcaagtgatgatttaccaaaaatgtttattgaatcggatccaggattcttttccaatgctattgttgaaggcgccaagaagtttcctaatactgaatttgtcaaagtaaaaggtcttcatttttcgcaagaagatgcacctgatgaaatgggaaaatatatcaaatcgttcgttgagcgagttctcaaaaatgaacaataa

SEQ ID NO: 56 - DNA, 2163 bp, Artificial Sequence
  Modified human neuropeptide Y receptor Y1
atgaattcaacattattttcccaggttgaaaatcattcagtccactctaatttctcagagaagaatgcccagcttctggcttttgaaaatgatgattgtcatctgcccttggccatgatatttaccttagctcttgcttatggagctgtgatcattcttggtgtctctggaaacctggccttgatcataatcatcttgaaacaaaaggagatgagaaatgttaccaacatcctgattgtgaacctttccttctcagacttgcttgttgccatcatgtgtctcccctttacatttgtctacacattaatggaccactgggtctttggtgaggcgatgtgtaagttgaatccttttgtgcaatgtgtttcaatcactgtgtccattttctctctggttctcattgctgtggaacgacatcagctgataatcaaccctcgagggtggagaccaaataatagacatgcttatgtaggtattgctgtgatttgggtccttgctgtggcttcttctttgcctttcctgatctaccaagtaatgactgatgagccgttccaaaatgtaacacttgatgcgtacaaagacaaatacgtgtgctttgatcaatttccatcggactctcataggttgtcttataccactctcctcttggtgctgcagtattttggtccactttgttttatatttatttgcggtaccagggtctttcaggaggccaaaaggcagctccagaagattgacaaatctgagggccgcttccatgtccagaaccttagccaggtggagcaggatgggcggacggggcatggactccgcagatcttccaagttctgctccagggaaaagaaagcagccaaaaccttggaattcattgtggtagcatttgcagtctgctggctccctcttaccatctttaacactgtgtttgattggaatcatcagatcattgctacctgcaaccacaatctgttattcctgctctgccacctcacagcaatgatatccacttgtgtcaaccccatattttatgggttcctgaacaaaaacttccagagagacttgcagttcttcttcaacttttgtgatttccggtctcgggatgatgattatgaaacaatagccatgtccacgatgcacacagatgtttccaaaacttctttgaagcaagcaagcccagtcgcatttaaaaaaatcaacaacaatgatgataatgaaaaaatcgagaattccatgacttcgaaagtttatgatccagaacaaaggaaacggatgataactggtccgcagtggtgggccagatgtaaacaaatgaatgttcttgattcatttattaattattatgattcagaaaaacatgcagaaaatgctgttatttttttacatggtaacgcggcctcttcttatttatggcgacatgttgtgccacatattgagccagtagcgcggtgtattataccagatcttattggtatgggcaaatcaggcaaatctggtaatggttcttataggttacttgatcattacaaatatcttactgcatggtttgaacttcttaatttaccaaagaagatcaattttgtcggccatgattggggtgcttgtttggcatttcattatagctatgagcatcaagataagatcaaagcaatagttcacgctgaaagtgtagtagatgtgattgaatcatgggatgaatggcctgatattgaagaagatattgcgttgatcaaatctgaagaaggagaaaaaatggttttggagaataacttcttcgtggaaaccatgttgccatcaaaaatcatgagaaagttagaaccagaagaatttgcagcatatcttgaaccattcaaagagaaaggtgaagttcgtcgtccaacattatcatggcctcgtgaaatcccgttagtaaaaggtggtaaacctgacgttgtacaaattgttaggaattataatgcttatctacgtgcaagtgatgatttaccaaaaatgtttattgaatcggatccaggattcttttccaatgctattgttgaaggcgccaagaagtttcctaatactgaatttgtcaaagtaaaaggtcttcatttttcgcaagaagatgcacctgatgaaatgggaaaatatatcaaatcgttcgttgagcgagttctcaaaaatgaacaataa

SEQ ID NO: 57 - DNA, 2109 bp, Artificial Sequence
  Modified human angiotensin II receptor 1
atgattctcaactcttctactgaagatggtattaaaagaatccaagatgattgtcccaaagctggaaggcataattacatatttgtcatgattcctactttatacagtatcatctttgtggtgggaatatttggaaacagcttggtggtgatagtcatttacttttatatgaagctgaagactgtggccagtgtttttcttttgaatttagcactggctgacttatgctttttactgactttgccactatgggctgtctacacagctatggaataccgctggccctttggcaattacctatgtaagattgcttcagccagcgtcagtttcaacctgtacgctagtgtgtttctactcacgtgtctcagcattgatcgatacctggctattgttcacccaatgaagtcccgccttcgacgcacaatgcttgtagccaaagtcacctgcatcatcatttggctgctggcaggcttggccagtttgccagctataatccatcgaaatgtatttttcattgagaacaccaatattacagtttgtgctttccattatgagtcccaaaattcaacccttccgatagggctgggcctgaccaaaaatatactgggtttcctgtttccttttctgatcattcttacaagtggtaccagggtctttcaggaggccaaaaggcagctccagaagattgacaaatctgagggccgcttccatgtccagaaccttagccaggtggagcaggatgggcggacggggcatggactccgcagatcttccaagttctgctccagggaaaagaaagcagccaaaaccttggaattcattgtgcttttctttttcttttcctggattccccaccaaatattcacttttctggatgtattgattcaactaggcatcatacgtgactgtagaattgcagatattgtggacacggccatgcctatcaccatttgtatagcttattttaacaattgcctgaatcctcttttttatggctttctggggaaaaaatttaaaagatattttctccagcttctaaaatatattcccccaaaagccaaatcccactcaaacctttcaacaaaaatgagcacgctttcctaccgcccctcagataatgtaagctcatccaccaagaagcctgcaccatgttttgaggttgaggagaattccatgacttcgaaagtttatgatccagaacaaaggaaacggatgataactggtccgcagtggtgggccagatgtaaacaaatgaatgttcttgattcatttattaattattatgattcagaaaaacatgcagaaaatgctgttatttttttacatggtaacgcggcctcttcttatttatggcgacatgttgtgccacatattgagccagtagcgcggtgtattataccagatcttattggtatgggcaaatcaggcaaatctggtaatggttcttataggttacttgatcattacaaatatcttactgcatggtttgaacttcttaatttaccaaagaagatcaattttgtcggccatgattggggtgcttgtttggcatttcattatagctatgagcatcaagataagatcaaagcaatagttcacgctgaaagtgtagtagatgtgattgaatcatgggatgaatggcctgatattgaagaagatattgcgttgatcaaatctgaagaaggagaaaaaatggttttggagaataacttcttcgtggaaaccatgttgccatcaaaaatcatgagaaagttagaaccagaagaatttgcagcatatcttgaaccattcaaagagaaaggtgaagttcgtcgtccaacattatcatggcctcgtgaaatcccgttagtaaaaggtggtaaacctgacgttgtacaaattgttaggaattataatgcttatctacgtgcaagtgatgatttaccaaaaatgtttattgaatcggatccaggattcttttccaatgctattgttgaaggcgccaagaagtttcctaatactgaatttgtcaaagtaaaaggtcttcatttttcgcaagaagatgcacctgatgaaatgggaaaatatatcaaatcgttcgttgagcgagttctcaaaaatgaacaataa

SEQ ID NO: 58 - DNA, 2220 bp, Artificial Sequence
  Modified human neurotensin receptor 1
atgcgcctcaacagctccgcgccgggaaccccgggcacgccggccgccgaccccttccagcgggcgcaggccggactggaggaggcgctgctggccccgggcttcggcaacgcttcgggcaacgcgtcggagcgcgtcctggcggcacccagcagcgagctggacgtgaacaccgacatctactccaaagtgctggtgaccgccgtgtacctggcgctcttcgtggtgggcacggtgggcaacacggtgacggcgttcacgctggcgcggaagaagtcgctgcagagcctgcagagcacggtgcattaccacctgggcagcctggcgctgtccgacctgctcaccctgctgctggccatgcccgtggagctgtacaacttcatctgggtgcaccacccctgggccttcggcgacgccggctgccgcggctactacttcctgcgcgacgcctgcacctacgccacggccctcaacgtggccagcctgagtgtggagcgctacctggccatctgccaccccttcaaggccaagaccctcatgtcccgaagccgcaccaagaagttcatcagcgccatctggctcgcctcggccctgctgacggtgcctatgctgttcaccatgggcgagcagaaccgcagcgccgacggccagcacgccggcggcctggtgtgcacccccaccatccacactgccaccgtcaaggtcgtcatacaggtcaacaccttcatgtccttcatattccccatggtggtcatctcggtcctgggtaccagggtctttcaggaggccaaaaggcagctccagaagattgacaaatctgagggccgcttccatgtccagaaccttagccaggtggagcaggatgggcggacggggcatggactccgcagatcttccaagttctgctccagggaaaagaaagcagccaaaaccttggaattcgtggtcatcgcctttgtggtctgctggctgccctaccacgtgcggcgcctcatgttctgctacatctcggatgagcagtggactccgttcctctatgacttctaccactacttctacatggtgaccaacgcactcttctacgtcagctccaccatcaaccccatcctgtacaacctcgtctctgccaacttccgccacatcttcctggccacactggcctgcctctgcccggtgtggcggcgcaggaggaagaggccagccttctcgaggaaggccgacagcgtgtccagcaaccacaccctctccagcaatgccacccgcgagacgctgtacgagaattccatgacttcgaaagtttatgatccagaacaaaggaaacggatgataactggtccgcagtggtgggccagatgtaaacaaatgaatgttcttgattcatttattaattattatgattcagaaaaacatgcagaaaatgctgttatttttttacatggtaacgcggcctcttcttatttatggcgacatgttgtgccacatattgagccagtagcgcggtgtattataccagatcttattggtatgggcaaatcaggcaaatctggtaatggttcttataggttacttgatcattacaaatatcttactgcatggtttgaacttcttaatttaccaaagaagatcaattttgtcggccatgattggggtgcttgtttggcatttcattatagctatgagcatcaagataagatcaaagcaatagttcacgctgaaagtgtagtagatgtgattgaatcatgggatgaatggcctgatattgaagaagatattgcgttgatcaaatctgaagaaggagaaaaaatggttttggagaataacttcttcgtggaaaccatgttgccatcaaaaatcatgagaaagttagaaccagaagaatttgcagcatatcttgaaccattcaaagagaaaggtgaagttcgtcgtccaacattatcatggcctcgtgaaatcccgttagtaaaaggtggtaaacctgacgttgtacaaattgttaggaattataatgcttatctacgtgcaagtgatgatttaccaaaaatgtttattgaatcggatccaggattcttttccaatgctattgttgaaggcgccaagaagtttcctaatactgaatttgtcaaagtaaaaggtcttcatttttcgcaagaagatgcacctgatgaaatgggaaaatatatcaaatcgttcgttgagcgagttctcaaaaatgaacaataa

SEQ ID NO: 59 - DNA, 2022 bp, Artificial Sequence
  human melanocortin 4 receptor
atggtgaactccacccaccgtgggatgcacacttctctgcacctctggaaccgcagcagttacagactgcacagcaatgccagtgagtcccttggaaaaggctactctgatggagggtgctacgagcaactttttgtctctcctgaggtgtttgtgactctgggtgtcatcagcttgttggagaatatcttagtgattgtggcaatagccaagaacaagaatctgcattcacccatgtactttttcatctgcagcttggctgtggctgatatgctggtgagcgtttcaaatggatcagaaaccattatcatcaccctattaaacagtacagatacggatgcacagagtttcacagtgaatattgataatgtcattgactcggtgatctgtagctccttgcttgcatccatttgcagcctgctttcaattgcagtggacaggtactttactatcttctatgctctccagtaccataacattatgacagttaagcgggttgggatcagcataagttgtatctgggcagcttgcacggtttcaggcattttgttcatcatttactcagatagtagtgctgtcatcatctgcctcatcaccatgttcttcaccatgctggctctcatggcttctctctatgtccacatgttcctgggtaccagggtctttcaggaggccaaaaggcagctccagaagattgacaaatctgagggccgcttccatgtccagaaccttagccaggtggagcaggatgggcggacggggcatggactccgcagatcttccaagttctgctccagggaaaagaaagcagccaaaaccttggaattcctgattggcgtctttgttgtctgctgggccccattcttcctccacttaatattctacatctcttgtcctcagaatccatattgtgtgtgcttcatgtctcactttaacttgtatctcatactgatcatgtgtaattcaatcatcgatcctctgatttatgcactccggagtcaagaactgaggaaaaccttcaaagagatcatctgttgctatcccctgggaggcctttgtgacttgtctagcagatatgagaattccatgacttcgaaagtttatgatccagaacaaaggaaacggatgataactggtccgcagtggtgggccagatgtaaacaaatgaatgttcttgattcatttattaattattatgattcagaaaaacatgcagaaaatgctgttatttttttacatggtaacgcggcctcttcttatttatggcgacatgttgtgccacatattgagccagtagcgcggtgtattataccagatcttattggtatgggcaaatcaggcaaatctggtaatggttcttataggttacttgatcattacaaatatcttactgcatggtttgaacttcttaatttaccaaagaagatcaattttgtcggccatgattggggtgcttgtttggcatttcattatagctatgagcatcaagataagatcaaagcaatagttcacgctgaaagtgtagtagatgtgattgaatcatgggatgaatggcctgatattgaagaagatattgcgttgatcaaatctgaagaaggagaaaaaatggttttggagaataacttcttcgtggaaaccatgttgccatcaaaaatcatgagaaagttagaaccagaagaatttgcagcatatcttgaaccattcaaagagaaaggtgaagttcgtcgtccaacattatcatggcctcgtgaaatcccgttagtaaaaggtggtaaacctgacgttgtacaaattgttaggaattataatgcttatctacgtgcaagtgatgatttaccaaaaatgtttattgaatcggatccaggattcttttccaatgctattgttgaaggcgccaagaagtttcctaatactgaatttgtcaaagtaaaaggtcttcatttttcgcaagaagatgcacctgatgaaatgggaaaatatatcaaatcgttcgttgagcgagttctcaaaaatgaacaataa

SEQ ID NO: 60 - DNA, 2457 bp, Artificial Sequence
  Modified human glucagon-like peptide 1 receptor
atggccggcgcccccggcccgctgcgccttgcgctgctgctgctcgggatggtgggcagggccggcccccgcccccagggtgccactgtgtccctctgggagacggtgcagaaatggcgagaataccgacgccagtgccagcgctccctgactgaggatccacctcctgccacagacttgttctgcaaccggaccttcgatgaatacgcctgctggccagatggggagccaggctcgttcgtgaatgtcagctgcccctggtacctgccctgggccagcagtgtgccgcagggccacgtgtaccggttctgcacagctgaaggcctctggctgcagaaggacaactccagcctgccctggagggacttgtcggagtgcgaggagtccaagcgaggggagagaagctccccggaggagcagctcctgttcctctacatcatctacacggtgggctacgcactctccttctctgctctggttatcgcctctgcgatcctcctcggcttcagacacctgcactgcaccaggaactacatccacctgaacctgtttgcatccttcatcctgcgagcattgtccgtcttcatcaaggacgcagccctgaagtggatgtatagcacagccgcccagcagcaccagtgggatgggctcctctcctaccaggactctctgagctgccgcctggtgtttctgctcatgcagtactgtgtggcggccaattactactggctcttggtggagggcgtgtacctgtacacactgctggccttctcggtcttctctgagcaatggatcttcaggctctacgtgagcataggctggggtgttcccctgctgtttgttgtcccctggggcattgtcaagtacctctatgaggacgagggctgctggaccaggaactccaacatgaactactggctcattatccggctgcccattctctttggcattggggtgaacttcctcatctttgttcgggtcatctgcggtaccagggtctttcaggaggccaaaaggcagctccagaagattgacaaatctgagggccgcttccatgtccagaaccttagccaggtggagcaggatgggcggacggggcatggactccgcagatcttccaagttctgctccagggaaaagaaagcagccaaaaccttggaattcagacttgccaagtccacgctgacactcatccccctgctggggactcatgaggtcatctttgcctttgtgatggacgagcacgcccgggggaccctgcgcttcatcaagctgtttacagagctctccttcacctccttccaggggctgatggtggccatcttatactgctttgtcaacaatgaggtccagctggaatttcggaagagctgggagcgctggcggcttgagcacttgcacatccagagggacagcagcatgaagcccctcaagtgtcccaccagcagcctgagcagtggagccacggcgggcagcagcatgtacacagccacttgccaggcctcctgcagcgagaattccatgacttcgaaagtttatgatccagaacaaaggaaacggatgataactggtccgcagtggtgggccagatgtaaacaaatgaatgttcttgattcatttattaattattatgattcagaaaaacatgcagaaaatgctgttatttttttacatggtaacgcggcctcttcttatttatggcgacatgttgtgccacatattgagccagtagcgcggtgtattataccagatcttattggtatgggcaaatcaggcaaatctggtaatggttcttataggttacttgatcattacaaatatcttactgcatggtttgaacttcttaatttaccaaagaagatcaattttgtcggccatgattggggtgcttgtttggcatttcattatagctatgagcatcaagataagatcaaagcaatagttcacgctgaaagtgtagtagatgtgattgaatcatgggatgaatggcctgatattgaagaagatattgcgttgatcaaatctgaagaaggagaaaaaatggttttggagaataacttcttcgtggaaaccatgttgccatcaaaaatcatgagaaagttagaaccagaagaatttgcagcatatcttgaaccattcaaagagaaaggtgaagttcgtcgtccaacattatcatggcctcgtgaaatcccgttagtaaaaggtggtaaacctgacgttgtacaaattgttaggaattataatgcttatctacgtgcaagtgatgatttaccaaaaatgtttattgaatcggatccaggattcttttccaatgctattgttgaaggcgccaagaagtttcctaatactgaatttgtcaaagtaaaaggtcttcatttttcgcaagaagatgcacctgatgaaatgggaaaatatatcaaatcgttcgttgagcgagttctcaaaaatgaacaataa

SEQ ID NO: 61 - DNA, 1983 bp, Artificial Sequence
  Modified human adenosine A1 receptor
atgccgccctccatctcagctttccaggccgcctacatcggcatcgaggtgctcatcgccctggtctctgtgcccgggaacgtgctggtgatctgggcggtgaaggtgaaccaggcgctgcgggatgccaccttctgcttcatcgtgtcgctggcggtggctgatgtggccgtgggtgccctggtcatccccctcgccatcctcatcaacattgggccacagacctacttccacacctgcctcatggttgcctgtccggtcctcatcctcacccagagctccatcctggccctgctggcaattgctgtggaccgctacctccgggtcaagatccctctccggtacaagatggtggtgaccccccggagggcggcggtggccatagccggctgctggatcctctccttcgtggtgggactgacccctatgtttggctggaacaatctgagtgcggtggagcgggcctgggcagccaacggcagcatgggggagcccgtgatcaagtgcgagttcgagaaggtcatcagcatggagtacatggtctacttcaacttctttgtgtgggtgctgcccccgcttctcctcatggtcctcatcggtaccagggtctttcaggaggccaaaaggcagctccagaagattgacaaatctgagggccgcttccatgtccagaaccttagccaggtggagcaggatgggcggacggggcatggactccgcagatcttccaagttctgctccagggaaaagaaagcagccaaaaccttggaattcatcctcttcctctttgccctcagctggctgcctttgcacatcctcaactgcatcaccctcttctgcccgtcctgccacaagcccagcatccttacctacattgccatcttcctcacgcacggcaactcggccatgaaccccattgtctatgccttccgcatccagaagttccgcgtcaccttccttaagatttggaatgaccatttccgctgccagcctgcacctcccattgacgaggatctcccagaagagaggcctgatgacgagaattccatgacttcgaaagtttatgatccagaacaaaggaaacggatgataactggtccgcagtggtgggccagatgtaaacaaatgaatgttcttgattcatttattaattattatgattcagaaaaacatgcagaaaatgctgttatttttttacatggtaacgcggcctcttcttatttatggcgacatgttgtgccacatattgagccagtagcgcggtgtattataccagatcttattggtatgggcaaatcaggcaaatctggtaatggttcttataggttacttgatcattacaaatatcttactgcatggtttgaacttcttaatttaccaaagaagatcaattttgtcggccatgattggggtgcttgtttggcatttcattatagctatgagcatcaagataagatcaaagcaatagttcacgctgaaagtgtagtagatgtgattgaatcatgggatgaatggcctgatattgaagaagatattgcgttgatcaaatctgaagaaggagaaaaaatggttttggagaataacttcttcgtggaaaccatgttgccatcaaaaatcatgagaaagttagaaccagaagaatttgcagcatatcttgaaccattcaaagagaaaggtgaagttcgtcgtccaacattatcatggcctcgtgaaatcccgttagtaaaaggtggtaaacctgacgttgtacaaattgttaggaattataatgcttatctacgtgcaagtgatgatttaccaaaaatgtttattgaatcggatccaggattcttttccaatgctattgttgaaggcgccaagaagtttcctaatactgaatttgtcaaagtaaaaggtcttcatttttcgcaagaagatgcacctgatgaaatgggaaaatatatcaaatcgttcgttgagcgagttctcaaaaatgaacaataa

SEQ ID NO: 62 - DNA, 2370 bp, Artificial Sequence
  Modified human cannabinoid receptor 1
atgaagtcgatcctagatggccttgcagataccaccttccgcaccatcaccactgacctcctgtacgtgggctcaaatgacattcagtacgaagacatcaaaggtgacatggcatccaaattagggtacttcccacagaaattccctttaacttcctttaggggaagtcccttccaagagaagatgactgcgggagacaacccccagctagtcccagcagaccaggtgaacattacagaattttacaacaagtctctctcgtccttcaaggagaatgaggagaacatccagtgtggggagaacttcatggacatagagtgtttcatggtcctgaaccccagccagcagctggccattgcagtcctgtccctcacgctgggcaccttcacggtcctggagaacctcctggtgctgtgcgtcatcctccactcccgcagcctccgctgcaggccttcctaccacttcatcggcagcctggcggtggcagacctcctggggagtgtcatttttgtctacagcttcattgacttccacgtgttccaccgcaaagatagccgcaacgtgtttctgttcaaactgggtggggtcacggcctccttcactgcctccgtgggcagcctgttcctcacagccatcgacaggtacatatccattcacaggcccctggcctataagaggattgtcaccaggcccaaggccgtggtagcgttttgcctgatgtggaccatagccattgtgatcgccgtgctgcctctcctgggctggaactgcgagaaactgcaatctgtttgctcagacattttcccacacattgatgaaacctacctgatgttctggatcggggtcaccagcgtactgcttctgttcatcgtgggtaccagggtctttcaggaggccaaaaggcagctccagaagattgacaaatctgagggccgcttccatgtccagaaccttagccaggtggagcaggatgggcggacggggcatggactccgcagatcttccaagttctgctccagggaaaagaaagcagccaaaaccttggaattcatcctggtggtgttgatcatctgctggggccctctgcttgcaatcatggtgtatgatgtctttgggaagatgaacaagctcattaagacggtgtttgcattctgcagtatgctctgcctgctgaactccaccgtgaaccccatcatctatgctctgaggagtaaggacctgcgacacgctttccggagcatgtttccctcttgtgaaggcactgcgcagcctctggataacagcatgggggactcggactgcctgcacaaacacgcaaacaatgcagccagtgttcacagggccgcagaaagctgcatcaagagcacggtcaagattgccaaggtaaccatgtctgtgtccacagacacgtctgccgaggctctggagaattccatgacttcgaaagtttatgatccagaacaaaggaaacggatgataactggtccgcagtggtgggccagatgtaaacaaatgaatgttcttgattcatttattaattattatgattcagaaaaacatgcagaaaatgctgttatttttttacatggtaacgcggcctcttcttatttatggcgacatgttgtgccacatattgagccagtagcgcggtgtattataccagatcttattggtatgggcaaatcaggcaaatctggtaatggttcttataggttacttgatcattacaaatatcttactgcatggtttgaacttcttaatttaccaaagaagatcaattttgtcggccatgattggggtgcttgtttggcatttcattatagctatgagcatcaagataagatcaaagcaatagttcacgctgaaagtgtagtagatgtgattgaatcatgggatgaatggcctgatattgaagaagatattgcgttgatcaaatctgaagaaggagaaaaaatggttttggagaataacttcttcgtggaaaccatgttgccatcaaaaatcatgagaaagttagaaccagaagaatttgcagcatatcttgaaccattcaaagagaaaggtgaagttcgtcgtccaacattatcatggcctcgtgaaatcccgttagtaaaaggtggtaaacctgacgttgtacaaattgttaggaattataatgcttatctacgtgcaagtgatgatttaccaaaaatgtttattgaatcggatccaggattcttttccaatgctattgttgaaggcgccaagaagtttcctaatactgaatttgtcaaagtaaaaggtcttcatttttcgcaagaagatgcacctgatgaaatgggaaaatatatcaaatcgttcgttgagcgagttctcaaaaatgaacaataa

SEQ ID NO: 63 - DNA, 2082 bp, Artificial Sequence
  Modified human melanin-concentrating hormone receptor 1
atggacctggaagcctcgctgctgcccactggtcccaatgccagcaacacctctgatggccccgataacctcacttcggcaggatcacctcctcgcacggggagcatctcctacatcaacatcatcatgccttcggtgttcggcaccatctgcctcctgggcatcatcgggaactccacggtcatcttcgcggtcgtgaagaagtccaagctgcactggtgcaacaacgtccccgacatcttcatcatcaacctctcggtagtagatctcctctttctcctgggcatgcccttcatgatccaccagctcatgggcaatggggtgtggcactttggggagaccatgtgcaccctcatcacggccatggatgccaatagtcagttcaccagcacctacatcctgaccgccatggccattgaccgctacctggccactgtccaccccatctcttccacgaagttccggaagccctctgtggccaccctggtgatctgcctcctgtgggccctctccttcatcagcatcacccctgtgtggctgtatgccagactcatccccttcccaggaggtgcagtgggctgcggcatacgcctgcccaacccagacactgacctctactggttcaccctgtaccagtttttcctggcctttgccctgccttttgtggtcatcacagccgcaggtaccagggtctttcaggaggccaaaaggcagctccagaagattgacaaatctgagggccgcttccatgtccagaaccttagccaggtggagcaggatgggcggacggggcatggactccgcagatcttccaagttctgctccagggaaaagaaagcagccaaaaccttggaattcatctgtctggtcttctttgtgtgctgggcaccctactatgtgctacagctgacccagttgtccatcagccgcccgaccctcacctttgtctacttatacaatgcggccatcagcttgggctatgccaacagctgcctcaacccctttgtgtacatcgtgctctgtgagacgttccgcaaacgcttggtcctgtcggtgaagcctgcagcccaggggcagcttcgcgctgtcagcaacgctcagacggctgacgaggagaggacagaaagcaaaggcaccgagaattccatgacttcgaaagtttatgatccagaacaaaggaaacggatgataactggtccgcagtggtgggccagatgtaaacaaatgaatgttcttgattcatttattaattattatgattcagaaaaacatgcagaaaatgctgttatttttttacatggtaacgcggcctcttcttatttatggcgacatgttgtgccacatattgagccagtagcgcggtgtattataccagatcttattggtatgggcaaatcaggcaaatctggtaatggttcttataggttacttgatcattacaaatatcttactgcatggtttgaacttcttaatttaccaaagaagatcaattttgtcggccatgattggggtgcttgtttggcatttcattatagctatgagcatcaagataagatcaaagcaatagttcacgctgaaagtgtagtagatgtgattgaatcatgggatgaatggcctgatattgaagaagatattgcgttgatcaaatctgaagaaggagaaaaaatggttttggagaataacttcttcgtggaaaccatgttgccatcaaaaatcatgagaaagttagaaccagaagaatttgcagcatatcttgaaccattcaaagagaaaggtgaagttcgtcgtccaacattatcatggcctcgtgaaatcccgttagtaaaaggtggtaaacctgacgttgtacaaattgttaggaattataatgcttatctacgtgcaagtgatgatttaccaaaaatgtttattgaatcggatccaggattcttttccaatgctattgttgaaggcgccaagaagtttcctaatactgaatttgtcaaagtaaaaggtcttcatttttcgcaagaagatgcacctgatgaaatgggaaaatatatcaaatcgttcgttgagcgagttctcaaaaatgaacaataa

SEQ ID NO: 64 - DNA, 1941 bp, Artificial Sequence
  modified human GPR40 (insulin secretion)
atggacctgcccccgcagctctccttcggcctctatgtggccgcctttgcgctgggcttcccgctcaacgtcctggccatccgaggcgcgacggcccacgcccggctccgtctcacccctagcctggtctacgccctgaacctgggctgctccgacctgctgctgacagtctctctgcccctgaaggcggtggaggcgctagcctccggggcctggcctctgccggcctcgctgtgccccgtcttcgcggtggcccacttcttcccactctatgccggcgggggcttcctggccgccctgagtgcaggccgctacctgggagcagccttccccttgggctaccaagccttccggaggccgtgctattcctggggggtgtgcgcggccatctgggccctcgtcctgtgtcacctgggtctggtctttgggttggaggctccaggaggctggctggaccacagcaacacctccctgggcatcaacacaccggtcaacggctctccggtctgcctggaggcctgggacccggcctctgccggcccggcccgcttcagcctctctctcctgctcttttttctgcccttggccatcacagccttctgcggtaccagggtctttcaggaggccaaaaggcagctccagaagattgacaaatctgagggccgcttccatgtccagaaccttagccaggtggagcaggatgggcggacggggcatggactccgcagatcttccaagttctgctccagggaaaagaaagcagccaaaaccttggaattcgccctcctcacgctgctgctctgcgtaggaccctacaacgcctccaacgtggccagcttcctgtaccccaatctaggaggctcctggcggaagctggggctcatcacgggtgcctggagtgtggtgcttaatccgctggtgaccggttacttgggaaggggtcctggcctgaagacagtgtgtgcggcaagaacgcaagggggcaagtcccagaaggagaattccatgacttcgaaagtttatgatccagaacaaaggaaacggatgataactggtccgcagtggtgggccagatgtaaacaaatgaatgttcttgattcatttattaattattatgattcagaaaaacatgcagaaaatgctgttatttttttacatggtaacgcggcctcttcttatttatggcgacatgttgtgccacatattgagccagtagcgcggtgtattataccagatcttattggtatgggcaaatcaggcaaatctggtaatggttcttataggttacttgatcattacaaatatcttactgcatggtttgaacttcttaatttaccaaagaagatcaattttgtcggccatgattggggtgcttgtttggcatttcattatagctatgagcatcaagataagatcaaagcaatagttcacgctgaaagtgtagtagatgtgattgaatcatgggatgaatggcctgatattgaagaagatattgcgttgatcaaatctgaagaaggagaaaaaatggttttggagaataacttcttcgtggaaaccatgttgccatcaaaaatcatgagaaagttagaaccagaagaatttgcagcatatcttgaaccattcaaagagaaaggtgaagttcgtcgtccaacattatcatggcctcgtgaaatcccgttagtaaaaggtggtaaacctgacgttgtacaaattgttaggaattataatgcttatctacgtgcaagtgatgatttaccaaaaatgtttattgaatcggatccaggattcttttccaatgctattgttgaaggcgccaagaagtttcctaatactgaatttgtcaaagtaaaaggtcttcatttttcgcaagaagatgcacctgatgaaatgggaaaatatatcaaatcgttcgttgagcgagttctcaaaaatgaacaataa

SEQ ID NO: 65 - DNA, 1989 bp, Artificial Sequence
  Modified human GPCR2 (insulin secretion)
atggaatcatctttctcatttggagtgatccttgctgtcctggcctccctcatcattgctactaacacactagtggctgtggctgtgctgctgttgatccacaagaatgatggtgtcagtctctgcttcaccttgaatctggctgtggctgacaccttgattggtgtggccatctctggcctactcacagaccagctctccagcccttctcggcccacacagaagaccctgtgcagcctgcggatggcatttgtcacttcctccgcagctgcctctgtcctcacggtcatgctgatcacctttgacaggtaccttgccatcaagcagcccttccgctacttgaagatcatgagtgggttcgtggccggggcctgcattgccgggctgtggttagtgtcttacctcattggcttcctcccactcggaatccccatgttccagcagactgcctacaaagggcagtgcagcttctttgctgtatttcaccctcacttcgtgctgaccctctcctgcgttggcttcttcccagccatgctcctctttgtcttcggtaccagggtctttcaggaggccaaaaggcagctccagaagattgacaaatctgagggccgcttccatgtccagaaccttagccaggtggagcaggatgggcggacggggcatggactccgcagatcttccaagttctgctccagggaaaagaaagcagccaaaaccttggaattcctcattgggagctttgctctatcctggacccccttccttatcactggcattgtgcaggtggcctgccaggagtgtcacctctacctagtgctggaacggtacctgtggctgctcggcgtgggcaactccctgctcaacccactcatctatgcctattggcagaaggaggtgcgactgcagctctaccacatggccctaggagtgaagaaggtgctcacctcattcctcctctttctctcggccaggaattgtggcccagagaggcccagggaaagttcctgtcacatcgtcactatctccagctcagagtttgatggcgagaattccatgacttcgaaagtttatgatccagaacaaaggaaacggatgataactggtccgcagtggtgggccagatgtaaacaaatgaatgttcttgattcatttattaattattatgattcagaaaaacatgcagaaaatgctgttatttttttacatggtaacgcggcctcttcttatttatggcgacatgttgtgccacatattgagccagtagcgcggtgtattataccagatcttattggtatgggcaaatcaggcaaatctggtaatggttcttataggttacttgatcattacaaatatcttactgcatggtttgaacttcttaatttaccaaagaagatcaattttgtcggccatgattggggtgcttgtttggcatttcattatagctatgagcatcaagataagatcaaagcaatagttcacgctgaaagtgtagtagatgtgattgaatcatgggatgaatggcctgatattgaagaagatattgcgttgatcaaatctgaagaaggagaaaaaatggttttggagaataacttcttcgtggaaaccatgttgccatcaaaaatcatgagaaagttagaaccagaagaatttgcagcatatcttgaaccattcaaagagaaaggtgaagttcgtcgtccaacattatcatggcctcgtgaaatcccgttagtaaaaggtggtaaacctgacgttgtacaaattgttaggaattataatgcttatctacgtgcaagtgatgatttaccaaaaatgtttattgaatcggatccaggattcttttccaatgctattgttgaaggcgccaagaagtttcctaatactgaatttgtcaaagtaaaaggtcttcatttttcgcaagaagatgcacctgatgaaatgggaaaatatatcaaatcgttcgttgagcgagttctcaaaaatgaacaataa

(19) United States
(12) Patent Application Publication
(10) Pub. No.: US 2008/0009551 A1
(43) Pub. Date: Jan. 10, 2008

(54) Methods and Compositions for Identifying Modulators of G Protein-Coupled Receptors

(75) Inventors: Fu-Yue Zeng, San Diego, CA (US); Dominic Behan, San Diego, CA (US)

Correspondence Address: BOZICEVIC, FIELD & FRANCIS LLP;(ARENA PHARMACEUTICALS, INC.), 1900 UNIVERSITY AVENUE, SUITE 200, EAST PALO ALTO, CA, 94303, US

(73) Assignee: Arena Pharmaceuticals, Inc., San Diego, CA (US)

(21) Appl. No.: 10/554,521

(22) PCT Filed: Apr. 29, 2004
(86) PCT No.: PCT/US04/13543

§ 371 Date: Mar. 7, 2007

Related U.S. Application Data
(60) Provisional application No. 60/467,489, filed on Apr. 30, 2003.
(60) Provisional application No. 60/516,168, filed on Oct. 31, 2003.

(51) Int. Cl. A61K 35/00; A61P 43/00; C07H 21/00; C07K 14/00; C12Q 1/02; C12Q 1/68

(52) U.S. Cl. 514/789; 435/29; 435/6; 530/350; 536/25.3

(57) ABSTRACT

The subject invention provides methods for making ligand upregulatable G-protein coupled receptors (GPCRs). Ligand upregulatable GPCRs contain a modified TM5-IC3-TM6 segment, which provides for increased levels of the ligand upregulatable GPCR in a host cell in the presence of a ligand as compared to the absence of the ligand. The subject invention also provides assays for screening test compounds for a ligand of a GPCR using a ligand upregulatable GPCR. In these assays, a ligand upregulatable GPCR is contacted with a test compound, and an increase in the detectable amount of the ligand upregulatable GPCR in the host cell indicates that the test compound is a ligand of the GPCR. Said indicated ligands encompass modulators of the GPCR. Said modulators of the GPCR are particularly useful in treating GPCR-related conditions.

### BACKGROUND OF THE INVENTION

1. Field of the Invention

The present invention relates to methods and compositions for use in identifying whether a test compound is a ligand of a G protein-coupled receptor. GPCR ligands identified by said methods encompass modulators of the GPCR.

2. Background of the Invention

Although a number of receptor classes exist in humans, by far the most abundant and therapeutically relevant is represented by the G protein-coupled receptor (GPCR) class. It is estimated that there are some 30,000-40,000 genes within the human genome, and of these, approximately 2% are estimated to code for GPCRs.

GPCR signaling plays a vital role in a number of physiological contexts including, but not limited to, metabolism, inflammation, neuronal function, and cardiovascular function. For instance, by way of illustration and not limitation, GPCRs include receptors for biogenic amines, e.g., dopamine, epinephrine, histamine, glutamate, acetylcholine, and serotonin; for purines such as ADP and ATP; for the vitamin niacin; for lipid mediators of inflammation such as prostaglandins, lipoxins, platelet activating factor, and leukotrienes; for peptide hormones such as calcitonin, follicle stimulating hormone, gonadotropin releasing hormone, ghrelin, motilin, neurokinin, and oxytocin; for non-hormone peptides such as beta-endorphin, dynorphin A, Leu-enkephalin, and Met-enkephalin; for the non-peptide hormone melatonin; for polypeptides such as C5a anaphylatoxin and chemokines; for proteases such as thrombin, trypsin, and factor Xa; and for sensory signal mediators, e.g., retinal photopigments and olfactory stimulatory molecules.

GPCRs represent an important area for the development of pharmaceutical products: from approximately 20 of the 100 known GPCRs, approximately 60% of all prescription pharmaceuticals have been developed. For example, in 1999, of the top 100 brand name prescription drugs, the following drugs interact with GPCRs (the primary disease and/or disorder treated related to the drug is indicated in parentheses): Claritin® (allergies), Prozac® (depression), Vasotec® (hypertension), Paxil® (depression), Zoloft® (depression), Zyprexa® (psychotic disorder), Cozaar® (hypertension), Imitrex® (migraine), Zantac® (reflux), Propulsid® (reflux disease), Risperdal® (schizophrenia), Serevent® (asthma), Pepcid® (reflux), Gaster® (ulcers), Atrovent® (bronchospasm), Effexor® (depression), Depakote® (epilepsy), Cardura® (prostatic hypertrophy), Allegra® (allergies), Lupron® (prostate cancer), Zoladex® (prostate cancer), Diprivan® (anesthesia), BuSpar® (anxiety), Ventolin® (bronchospasm), Hytrin® (hypertension), Wellbutrin® (depression), Zyrtec®, (rhinitis), Plavix® (MI/stroke), Toprol-XL® (hypertension), Tenormin® (angina), Xalatan® (glaucoma), Singulair® (asthma), Diovan® (hypertension) and Harnal® (prostatic hyperplasia) (Med Ad News 1999 Data).

GPCRs share a common structural motif, having seven sequences of between 22 to 24 hydrophobic amino acids that form seven alpha helices, each of which spans the membrane (each span is identified by number, i.e., transmembrane-1 (TM1), transmembrane-2 (TM2), etc.). The transmembrane helices are joined by strands of amino acids between transmembrane-2 and transmembrane-3, transmembrane-4 and transmembrane-5, and transmembrane-6 and transmembrane-7 on the exterior, or “extracellular” side, of the cell membrane (these are referred to as “extracellular” regions 1, 2 and 3 (EC1, EC2 and EC3), respectively). The transmembrane helices are also joined by strands of amino acids between transmembrane-1 and transmembrane-2, transmembrane-3 and transmembrane-4, and transmembrane-5 and transmembrane-6 on the interior, or “intracellular” side, of the cell membrane (these are referred to as “intracellular” regions 1, 2 and 3 (IC1, IC2 and IC3), respectively). The “carboxy” (“C”) terminus of the receptor lies in the intracellular space within the cell, and the “amino” (“N”) terminus of the receptor lies in the extracellular space outside of the cell.

Generally, when a ligand binds with the receptor (often referred to as “activation” of the receptor), there is a change in the conformation of the receptor that facilitates coupling between the intracellular region and an intracellular “G-protein.” It has been reported that GPCRs are “promiscuous” with respect to G proteins, i.e., that a GPCR can interact with more than one G protein. See, Kenakin, T., 43 Life Sciences 1095 (1988). Although other G proteins exist, currently, Gq, Gs, Gi, Gz and Go are G proteins that have been identified. Coupling with Gq leads to an increase in intracellular IP3 concentration and an increase in intracellular Ca²⁺ concentration. Coupling to Gs leads to an increase in intracellular cAMP concentration. Coupling to Gi, Go, or Gz leads to a decrease in intracellular cAMP concentration. Ligand-activated GPCR coupling with a G-protein initiates a signaling cascade process (referred to as “signal transduction”). Under normal conditions, signal transduction ultimately results in cellular activation or cellular inhibition.

Under physiological conditions, GPCRs exist in the cell membrane in equilibrium between two different conformations: an “inactive” state and an “active” state. A receptor in an inactive state is unable to link to the intracellular signaling transduction pathway to initiate signal transduction leading to a biological response. Changing the receptor conformation to the active state allows linkage to the transduction pathway (via the G-protein) and produces a biological response.

A receptor may be placed in an active state by a ligand or a compound such as a drug. Alternatively, the amino acid sequence of the receptor may be altered to place the receptor in an active state by simulating the effect of a ligand binding to the receptor. Stabilization by such ligand-independent means is termed “constitutive receptor activation,” and the receptor is “constitutively activated.”

It follows from the foregoing that compounds that specifically bind to and modulate GPCRs are extremely desirable and, as such, there is a great need for methods and compositions for identifying such compounds. In particular, there is a great need for widely applicable, effective, high-throughput and sensitive assays for screening test compounds for the capacity to bind to a target GPCR of interest, wherein GPCR-binding compounds identified by said assays encompass modulators of the GPCR. This invention meets these, and other, needs with unpredictably high level of success.

Literature

Literature of interest includes: Ramsay et al., Detection of receptor ligands by monitoring selective stabilization of a Renilla luciferase-tagged, constitutively active mutant, G-protein-coupled receptor. Br J Pharmacol. 2001 133:315-23; Stevens et al., Resolution of inverse agonist-induced up-regulation from constitutive activity of mutants of the alpha1b-adrenoreceptor. Mol Pharmacol 2000 58:438-48; McLean et al., Visualizing differences in ligand regulation of wild-type and constitutively active mutant beta(2)-adrenoceptor-green fluorescent protein fusion proteins. Mol Pharmacol. 1999 56:1182-91; McLean et al., Generation and analysis of constitutively active and physically destabilized mutants of the human beta(1)-adrenoceptor. Mol Pharmacol. 2002 62:747-55; Samama et al., Ligand-induced overexpression of a constitutively active beta2-adrenergic receptor: pharmacological creation of a phenotype in transgenic mice. Proc Natl Acad Sci 1997 94:137-41; Samama et al., Negative antagonists promote an inactive conformation of the beta 2-adrenergic receptor. Mol Pharmacol. 1994 45:390-4; Ren et al., Constitutively active mutants of the alpha 2-adrenergic receptor. J Biol Chem. 1993 268:16483-7; Lefkowitz et al., Constitutive activity of receptors coupled to guanine nucleotide regulatory proteins. Trends Pharmacol Sci. 1993 14:303-7; Milligan et al., Inverse agonism at adrenergic and opioid receptors: studies with wild type and constitutively active mutant receptors. Receptors Channels. 1997 5:209-13; Pei et al. A constitutively active mutant beta 2-adrenergic receptor is constitutively desensitized and phosphorylated. Proc Natl Acad Sci 1994 91:2699-702; Samama et al., A mutation-induced activated state of the beta 2-adrenergic receptor. Extending the ternary complex model. J Biol Chem. 1993 268:4625-36; Parnot et al., Lessons from constitutively active mutants of G protein-coupled receptors. Trends Endocrinol Metab. 2002 13:336-43; Teitler et al, Constitutive activity of G-protein coupled receptors: emphasis on serotonin receptors. Curr Top Med Chem. 2002 2:529-38; Itoh et al, Free fatty acids regulate insulin secretion from pancreatic β cells through GPR40. Nature 2003 422:173-6; Stevens et al., Mol Pharmacol (2000) 58:438-48; PCT publication WO 02/44362; U.S. Pat. No. 6,221,660; and PCT publication WO 98/46995; Vassilatis et al, The G protein-coupled receptor repertoires of human and mouse. Proc Natl Acad Sci 2003 100:4903-8; Cello et al, Chemical synthesis of poliovirus cDNA: generation of infectious virus in the absence of natural template. Science 2002 297:1016-8.

### SUMMARY OF THE INVENTION

The subject invention features methods for making a G protein-coupled receptor (GPCR) upregulatable by ligand. The ligand upregulatable GPCR comprises a modified TM5-IC3-TM6 segment which provides for increased levels of the GPCR in the presence of a ligand to the GPCR as compared to in the absence of the ligand. The invention also features isolated polynucleotide comprising nucleotide sequence encoding a ligand upregulatable GPCR, and compositions related thereto. The subject invention also features methods of identifying whether a test compound is a ligand of a GPCR, the methods involving contacting a test compound with a ligand upregulatable GPCR. GPCR ligands identified by said methods encompass modulators of the GPCR.

The subject invention provides screening methods that can detect GPCR ligands encompassing modulators of the GPCR, at significantly less cost and with fewer steps than conventional secondary messenger assays. In most embodiments, the assay is non-radioactive, highly sensitive, and amenable to very high-throughput format.

In a first aspect, the invention provides a method for making a ligand upregulatable GPCR. In general, the method involves providing a substituted GPCR by modifying the TM5-IC3-TM6 segment of a parental GPCR such that the TM5-IC3-TM6 segment comprises substitution of the amino acid sequence of a GPCR upregulating cassette (GURC). In certain embodiments, the method further involves (a) producing said substituted GPCR in a host cell; and (b) comparing a detectable level of said substituted GPCR in the presence of a ligand to a detectable level of said substituted GPCR in the absence of the ligand; wherein a substituted GPCR that is detectable at a higher level in the host cell in the presence of the ligand than in the absence of the ligand is a ligand upregulatable GPCR

In certain embodiments, the ligand of step (b) is a native ligand for the parental GPCR.

In certain embodiments, the ligand of step (b) is a non-native ligand for the parental GPCR

In certain embodiments, the substituted GPCR is on the surface of said host cell.

In certain embodiments, the parental GPCR is not a β2-adrenergic receptor or variant thereof.

In certain embodiments the parental GPCR is not an adrenergic receptor (adrenoreceptor) or variant thereof. The α-type adrenoreceptors (e.g. α_{1A}, α_{1B} or α_{1C} adrenoreceptors) and β-type adrenoreceptors (e.g. β₁, β₂, or β₃ adrenoreceptors) are discussed in Singh et al., J. Cell Phys. 189:257-265, 2001.

In certain embodiments said comparing is quantitative.

In certain embodiments, the parental GPCR is a native GPCR or an altered native GPCR.

In certain embodiments, the parental GPCR is a known GPCR or an altered known GPCR.

In certain embodiments, the parental GPCR is an orphan GPCR or an altered orphan GPCR.

In certain embodiments, the parental GPCR is a liganded-orphan GPCR or an altered liganded-orphan GPCR.

In certain embodiments the parental GPCR is selected from the group consisting of (native or altered) purinergic receptor, vitamin receptor, lipid receptor, peptide hormone receptor, non-hormone peptide receptor, non-peptide hormone receptor, polypeptide receptor, protease receptor, receptor for sensory signal mediator, and biogenic amine receptor that is not a β2-adrenergic receptor. In certain embodiments, said biogenic amine receptor is not an adrenoreceptor.

In certain embodiments said GPCR upregulating cassette comprises the sequence of SEQ ID NO:1 at least proximal to a C-terminal end of the GPCR upregulating cassette.

In certain embodiments, the N-terminus of the substituted GURC amino acid sequence is juxtaposed to a first amino acid identical to or 1, 2, 3, 4, 5, 6, 7, 8, 9, or 10 residues N-terminal or C-terminal to the amino acid nine residues C terminal to a proline in TM5 of the parental GPCR, and the C-terminus of the substituted GURC amino acid sequence is juxtaposed to a second amino acid identical to or 1, 2, 3, 4, 5, 6, 7, 8, 9 or 10 residues N-terminal or C-terminal to the amino acid twelve residues N-terminal to a proline in TM6 of the parental GPCR.

In certain embodiments the N-terminus of the substituted GURC amino acid sequence is juxtaposed to a first amino acid nine residues C-terminal to a proline in TM5 of the parental GPCR, and the C-terminus of the substituted GURC amino acid sequence is juxtaposed to a second amino acid twelve residues N-terminal to a proline in TM6 of the parental GPCR.

In certain embodiments the two amino acids of the parental GPCR N-terminally flanking the substituted GURC are mutated, e.g. to Gly-Thr (GT).

In certain embodiments the two amino acids of the parental GPCR C-terminally flanking to the substituted GURC are mutated, e.g. to Glu-Phe (EF).

In certain embodiments said GURC is at least 70% identical to the sequence of SEQ ID NO:2, comprises at least 10 contiguous amino acids of SEQ ID NO:2, or comprises the sequence of SEQ ID NO:2, or a variant thereof.

In certain embodiments said ligand upregulatable GPCR further comprises a reporter protein domain and said detecting step (b) is done by means of a reporter assay.

In certain embodiments said substituted GPCR is a luciferase fusion and said detecting step comprises determining the level of luciferase activity, e.g. wild type or modified Renilla luciferase activity.

In certain embodiments said luciferase is fused downstream of said substituted GPCR.

In certain embodiments said detecting step (b) is by immunodetection using an antibody that binds the ligand upregulatable GPCR.

In certain embodiments the parental GPCR is a (native or altered) mammalian, e.g., human GPCR.

In certain embodiments the parental GPCR is selected from the group consisting of (native or altered) cholinergic receptor, muscarinic 3; melanin-concentrating hormone receptor 2; cholinergic receptor, muscarinic 4; niacin receptor; histamine 4 receptor; ghrelin receptor; CXCR3 chemokine receptor; motilin receptor; 5-hydroxytryptamine (serotonin) receptor 2A; 5-hydroxytyptamine (serotonin) receptor 2B; 5-hydroxytryptamine (serotonin) receptor 2C; dopamine receptor D3; dopamine receptor D4; dopamine receptor D1; histamine receptor H2; histamine receptor H3; galanin receptor 1; neuropeptide Y receptor Y1; angiotensin II receptor 1; neurotensin receptor 1; melanocortin 4 receptor; glucagon-like peptide 1 receptor; adenosine A1 receptor; cannabinoid receptor 1; melanin-concentrating hormone receptor 1; GPR40; and GPCR2.

In a second aspect, the invention provides a method for making a nucleic acid encoding a ligand upregulatable GPCR made according to the first aspect. In general, the method involves providing a nucleic acid encoding a substituted GPCR by modifying the TM5-IC3-TM6 segment-encoding nucleic acid of a parental GPCR-encoding nucleic acid such that the TM5-IC3-TM6 segment comprises substitution of the nucleotide sequence of a GPCR upregulating cassette. In certain embodiments, the method further involves (a) producing said substituted GPCR in a host cell; and, (b) comparing a detectable level of said substituted GPCR in the presence of a ligand to a detectable level of said substituted GPCR in the absence of the ligand; wherein a nucleic acid encoding a substituted GPCR that is detectably present at higher level in the host cell in the presence of the ligand than in the absence of the ligand is a nucleic acid encoding a ligand upregulatable GPCR.

In a third aspect, the invention provides an isolated polynucleotide comprising nucleotide sequence encoding a ligand upregulatable GPCR made according to a method of the first aspect, and compositions related thereto.

In a fourth aspect, the invention provides a method of identifying whether a test compound is a ligand for a parental GPCR. In general, the method involves: (a) contacting a test compound with a ligand upregulatable GPCR made according to a method of the first aspect, which ligand upregulatable GPCR is expressed by a host cell; and (b) comparing a first detectable level of said ligand upregulatable GPCR in the presence of the test compound to a second detectable level of said ligand upregulatable GPCR in the absence of the test compound; wherein said first detectable level greater than said second detectable level indicates that the test compound is a ligand for the parental GPCR.

In certain embodiments said ligand upregulatable GPCR comprises a reporter protein and said detecting step (b) is done by means of a reporter assay.

In certain embodiments said substituted GPCR is a luciferase fusion and said comparing step comprises determining the level of luciferase activity, e.g. wild type or modified Renilla luciferase activity.

In certain embodiments said detecting step (b) is by immunodetection using an antibody that binds the ligand upregulatable GPCR.

In certain embodiments said identified ligand is a modulator of the parental GPCR.

In certain embodiments said modulator of the parental GPCR is an agonist, an inverse agonist, an antagonist or a partial agonist.

In certain embodiments said modulator of the parental GPCR is a selective modulator of the parental GPCR.

In certain embodiments, the host cell comprises an expression vector comprising a polynucleotide encoding the ligand upregulatable GPCR.

In a fifth aspect, the invention provides a method of preparing a composition which comprises identifying a ligand of a GPCR and then admixing a carrier and the ligand, wherein said ligand is identified by a method according to the fourth aspect.

In a sixth aspect, the invention features a method of modulating a GPCR, said method comprising contacting a ligand for the GPCR identified according to a method of the fourth aspect with the GPCR, wherein said ligand is a modulator of the GPCR.

In a seventh aspect, the invention features a method of treating an individual for a GPCR-related disorder, said method comprising administering to said individual an effective amount of a ligand for the GPCR, wherein said ligand is identified according to a method of the fourth aspect and wherein said ligand is a modulator of the GPCR.

### BRIEF DESCRIPTION OF THE DRAWINGS

FIG. 1 is a schematic figure showing the structure of G-protein coupled receptors, with numbers assigned to transmembrane regions (TM1, TM2, TM3, TM4, TM5, TM6, and TM7), intracellular regions (IC1, IC2, and IC3), and extracellular regions (EC1, EC2, and EC3).

FIG. 2 is a table showing 28 exemplary G-protein coupled receptors suitable for use in the subject methods. The table has three columns: GPCR ID (N1-N28), which is an identifier for the GPCR, the name of the GPCR, and the Accession No. of the GPCR in GenBank. These GenBank entries, including the polynucleotides, polypeptides and other features described therein, are hereby specifically incorporated by reference to their accession numbers.

FIG. 3 shows exemplary polynucleotide and polypeptide sequences of the GPCR upregulating cassettes (“GURCs”) used in the subject methods. SEQ ID NOS: 2-8 are the amino acid sequences of representative GURCs, and SEQ ID NO:9 is a representative GURC-encoding nucleic acid. Underlined residues correspond to amino acids that are variant as compared to the corresponding amino acid in GURC1. The GURCs identified by SEQ ID NOS: 2-8 may be flanked by “GT” and “EF” amino acids, as shown in the polypeptides of described in FIG. 4. In many embodiments, a GURC of the present invention contains the sequence “SREKKAAK” (SEQ ID NO:1) proximal to its C-terminus.

FIG. 4 shows the polypeptide sequences (SEQ ID NOS 10-37) of 28 exemplary GPCRs that have been modified so as to become ligand upregulatable. For GPCR N1-N28, the parental GPCR is a native GPCR indicated in FIG. 2. In each of the sequences, the position of the GURC used to modify the GPCR is underlined, and the retained amino acids of the parental TM5 and TM6 transmembrane regions flanking either side of the substituted GURC are shown in bold. Each of the polypeptides is a Renilla luciferase fusion protein, with a three amino acid [Glu-Asn-Ser (“ENS”)] spacer region between the C-terminal amino acid of the GURC-modified GPCR and the N-terminal methionine of the luciferase. Within each of the sequences, the GURC used in each ligand upregulatable GPCR are flanked by modified “GT” and “EF” amino acids. Each of the modified GPCRs has been shown to be upregulatable by ligand, representative data for which is presented infra for a number of the GPCRs.

FIG. 5 is a schematic representation of wild-typeRm3 and substituted Rm3 receptors. (Top) The C-terminus of full-length Rm3 was fused in-frame to the N-terminal Met of Renilla luciferase (Rlu) through a spacer region of three amino acids Glu-Asn-Ser. (Bottom) Amino acids 250-494 of the wild type Rm3 were replaced with GURC1 with the introduction of KpnI and EcoRI sites, resulting in mutations of two amino acids N-terminally flanking (Tyr-Trp to Gly-Thr) or C-terminally flanking (Ser-Ala to Glu-Phe) the substituted GURC1. The modified Rm3(GURC1) was then linked in-frame to the N-terminal Met of the Renilla luciferase gene resulting in the insertion of three amino acids Glu-Asn-Ser.

FIG. 6 is a bar graph showing expression comparison of the wild-type Rm3, Rm3-Rlu and Rm3(GURC)-Rlu in COS-7 cells. The COS-7 cells were transiently transfected with wild-type Rm3, Rm3-Rlu and Rm3(GURC)-Rlu plasmids and cells were harvested after 48 h. Cell homogenates (20 μg/well) were incubated with 2 nM [³H]NMS at room temperature for 2 h in the absence of atropine (total binding) or in the presence of 10 □M atropine (non-specific binding). Data, expressed as specific CPM per well, are from one of typical experiment out of three experiments performed in triplicate.

FIGS. 7A-7D are graphs showing displacement of specific [³H]NMS binding to wild-type Rm3 and Rm3(GURC)-Rlu by agonist and antagonists. COS-7 cell homogenates (20 μg/well), transiently expressed the wild-type Rm3 (solid square) and Rm3(GURC)-Rlu (open triangle), were incubated in binding buffer with different concentrations of indicated ligands at room temperature for 30 min. [³H]NMS was added to final concentration of 500 pM and incubated at room temperature for another 1 h. Data are presented as percentage of control (in the absence of cold ligand, as 100%) from one of representative experiments performed in triplicate.

FIGS. 8A and 8B are bar graphs showing ligand-specific upregulation of Rm3(GURC)-Rlu transiently expressed in HEK293 cells. (A) HEK293 cells were transfected with Rm3(GURC)-Rlu. After 24 h transfection, cells (10 cm dish) were split into 96-well plate (50,000 cells per well) and 10 μM of each compound was added to the culture medium. Cells were cultured for a further 20-24 h prior to carrying out luciferase activity assays as described under the materials and methods. All data are expressed as mean ±S.E. of luciferase activity (RLU) from one out of six independent experiments, each carried out in duplicate. (B) Verification of ligand-mediated receptor upregulation by [³H]NMS binding assay. HEK293 cells stably expressing Rm3(GURC)-Rlu were incubated in culture medium for 20 h in the absence of (open bar) or in the presence of 1 μM atropine (solid bar). Cells were harvested, and extensively washed with ice-cold PBS to remove bound ligand. Binding was carried out with cell homogenates using [³H]NMS (2 nM) as described under the Materials and Methods. Data are presented as percentage of untreated cells (100%) and represent the mean ±S.E. of three independent experiments, each carried in duplicate.

FIG. 9 is a graph showing dose-dependent upregulation of Rm3(GURC)-Rlu by ligands. HEK293 cells stably expressing Rm3(GURC)-Rlu were split into 96-well plates and incubated with different concentrations of atropine (solid square), 4-DAMP (solid triangle), carbachol (open square) and oxotremorine M (open triangle) for a further 20 h period prior to carrying out luciferase activity assays as described under materials and methods. Data are expressed as percentage of control luciferase activity in the absence of ligand (as 100%) and represent observations from three independent experiments, each carried out in triplicate.

FIG. 10 is a bar chart showing one example of a LOPAC compound in 96-well format. Nine plates of LOPAC compound collection were screened at concentration of 2 μM. Results for one of these plates are showed here. Positive control (2 μM atropine) is set as 200% and negative control (0.5% DMSO) as 100%. Compounds that gave>140% activity are identified as follows: 1, atropine sulfate; 2, aminobenztropine; 3, benztropine; 4, As-1397; 5, 4-DAMP; 6, hexahydro-sila-difenidol; 7, hexahydro-sila-difenidol, ρ-fluoro analog; 8, himbacine; 9, ipratropium bromide; 10, pilocarpine; 11, pirenzepine; 12, quinidine sulfate; 13, (-) scopolamine; 14, (-) n-butyl scopolamine.

FIG. 11 is a table showing verification of hits identified in an upregulation assay screen by competition binding and functional NFAT-reporter gene assays. Receptor upregulation assay was performed in HEK293 cells stably expressing Rm3(GURC)-Rlu as described, Competition ligand binding assay was carried in membrane homogenates of COS-7 cells transiently transfected with the wild-type Rm3 as described in FIG. 3. NFAT-reporter gene assay was done in HEK293 cells transiently transfected with the wild-type Rm3 as described. Data represent one typical experiment performed in triplicate.

FIGS. 12A-12D are graphs showing upregulation of GPCRs modified according to the subject methods, in the presence of ligands for the GPCRs. In each of the graphs, the amount of GPCR is detected using a luciferase assay, and plotted on the graph as RLU (relative light units). Ligands and non-ligands are applied at various molarities, as shown by the axis marked “concentration (logM)”. A. Upregulation of 5 hydroxytryptamine (serotonin) receptor 2B. Serotonin is an agonist, and clozapine and metergoline are antagonists. B. Upregulation of 5 hydroxytryptamine (serotonin) receptor 2C. Serotonin and quipazine maleate are agonists; SB-206553, LY-53858 maleate and metergoline are antagonists; and atropine is not a ligand. C. Upregulation of neurotensin receptor 1. Neurotensin is an agonist, and 272349 and hexarelin are not ligands. D. Upregulation of the ghrelin receptor. All compounds are agonists.

FIGS. 13A-13D are graphs showing ligand specific upregulation of GPCRs modified according to the subject methods. In each of the graphs, the amount of GPCR is detected using a luciferase assay, and plotted on the graph as RLU (relative light units).

Ligands and non-ligands are applied at various molarities, as shown by the axis marked “concentration (logM)”. A. Upregulation of the dopamine receptor D1. Dopamine is an agonist, and the remainder of the compounds are antagonists. B. Upregulation of the dopamine receptor D4. All compounds are antagonists. C. Upregulation of the rat cholinergic receptor, muscarinic 3. Carbachol and oxotremoriline are agonists; 4-DAMP and atropine are antagonists. D. Upregulation of the pig adrenergic receptor, alpha-2A. UK14304 and clonidine are agonists; yohimbine and rauwolscine are antagonists.

FIGS. 14A and 14B are graphs showing ligand specific upregulation of the histamine receptor H3. A. Bar graph showing upregulation of the histamine receptor H3 modified using the subject methods. Imetit, methyl-histamine are agonists; clobenpropit, thioperamide and iodophenopropit are antagonists; other compounds are not ligands. All compounds were used at a concentration of 2 μM. Asterisk (*) indicate ligands. B. Concentration response graph for the histamine receptor H3 and four compounds. FIGS. 15A and 15B are graphs showing receptor-specific upregulation by niacin receptor ligand. A. Graph showing dose-dependent upregulation of the niacin receptor in response to methyl nicotinate, nicotinic acid, NAADP-β, atropine, and ATII peptide. The former three compounds are agonists of the niacin receptor; the latter two compounds are not ligands of the niacin receptor. B. Graph showing lack of dose-dependent upregulation of the β2 adrenergic receptor in response to methyl nicotinate, DAD and NAADP-β. These compounds are not ligands for the β2 adrenergic receptor.

FIGS. 16A and 16B are graphs showing that niacin upregulation of the modified niacin receptor is dependent on the presence of the GPCR upregulating cassette. Compounds were tested for two Rlu-modified niacin receptors: one additionally containing a GURC (FIG. 16A), and the other not containing a GURC (FIG. 16B). Only the niacin receptor containing the GURC was upregulated in the presence of a ligand for the receptor.

### DEFINITIONS

Before the present invention is further described, it is to be understood that this invention is not limited to particular embodiments described, as such may of course vary. It is also to be understood that the terminology used herein is for the purpose of describing particular embodiments only, and is not intended to be limiting. Unless defined otherwise, all technical and scientific terms used herein have the same meaning as commonly understood by one of ordinary skill in the art to which this invention belongs.

Where a range of values is provided, it is understood that each intervening value; to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Throughout this application, various publications, patents and published patent applications are cited. The disclosures of these publications, patents and published patent applications referenced in this application are hereby incorporated by reference in their entirety into the present disclosure. Citation herein by Applicant of a publication, patent, or published patent application is not an admission by Applicant of said publication, patent, or published patent application as prior art.

It must-be noted that as used herein and in the appended claims, the singular forms “a”, “and”, and “the” include plural referents unless the context clearly dictates otherwise. Thus, for example, reference to “an agent” includes a plurality of such agents, and reference to “the GPCR” includes reference to one or more GPCRs and equivalents thereof known to those skilled in the art, and so forth. It is further noted that the claims may be drafted to exclude any optional element. As such, this statement is intended to serve as antecedent basis for use of such exclusive terminology as “solely”, “only” and the like in connection with the recitation of claim elements, or the use of a “negative” limitation.

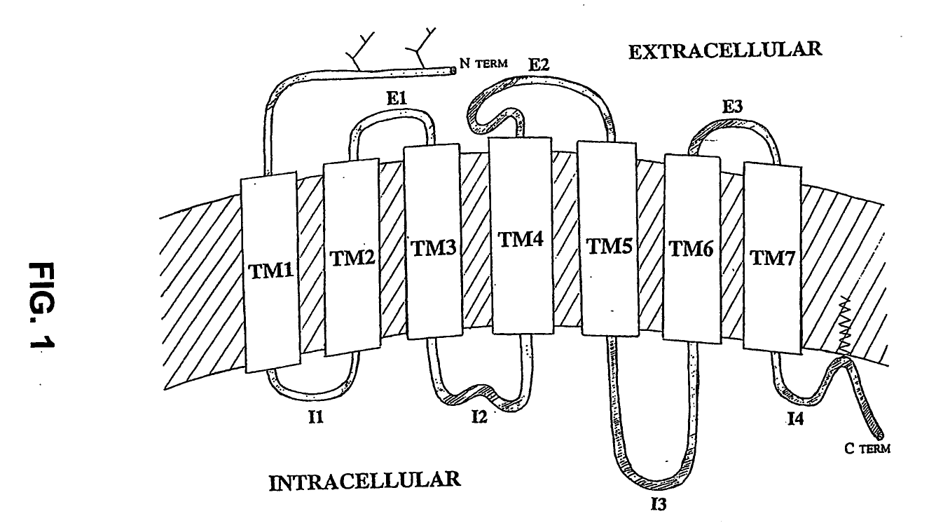

“G-protein coupled receptors”, or “GPCRs” are polypeptides that share a common structural motif, having seven regions of between 22 to 24 hydrophobic amino acids that form seven alpha helices, each of which spans a membrane [each span is identified by number, i.e., transmembrane-1 (TM1), transmembrane-2 (TM2), etc.]. The transmembrane helices are joined by regions of amino acids between transmembrane-2 and transmembrane-3, transmembrane-4 and transmembrane-5, and transmembrane-6 and transmembrane-7 on the exterior, or “extracellular” side, of the cell membrane [these are referred to as “extracellular” regions 1, 2 and 3 (EC1, EC2 and EC3), respectively]. The transmembrane helices are also joined by regions of amino acids between transmembrane-1 and transmembrane-2, transmembrane-3 and transmembrane-4, and transmembrane-5 and transmembrane-6 on the interior, or “intracellular” side, of the cell membrane [these are referred to as “intracellular” regions 1, 2 and 3 (IC1, IC2 and IC3), respectively]. The “carboxy” (“C”) terminus of the receptor lies in the intracellular space within the cell, and the “amino” (“N”) terminus of the receptor lies in the extracellular space outside of the cell. GPCR structure and classification is generally well known in the art, and further discussion of GPCRs may be found in Probst, DNA Cell Biol. 1992 11:1-20; Marchese et al Genomics 23: 609-618, 1994; and the following books: Jürgen Wess (Ed) Structure-Function Analysis of G Protein-Coupled Receptors published by Wiley-Liss (1st edition; Oct. 15, 1999); Kevin R. Lynch (Ed) Identification and Expression of G Protein-Coupled Receptors published by John Wiley & Sons (March 1998) and Tatsuya Haga (Ed), G Protein-Coupled Receptors, published by CRC Press (Sep. 24, 1999); and Steve Watson (Ed) G-Protein Linked Receptor Factsbook, published by Academic Press (1st edition; 1994). A schematic representation of a typical GPCR is shown in FIG. 1.

A “ligand upregulatable GPCR” is a modified GPCR the detectable amount of which in or on a cell increases upon binding of a ligand to the modified GPCR. The term encompasses GPCRs that are modified so as to be upregulated in the presence of a ligand such that the detectable amount of the modified GPCR (e.g., as detected by a signal of a reporter fused to the modified GPCR) is increased, as compared to the modified GPCR in the absence of the ligand.

A “native GPCR” is a GPCR that is produced by a mammal, or a pathogen of a mammal such as a virus, and may be an allelic variant of a GPCR that causes a GPCR-related condition. Detailed description of native GPCRs may be found in the On-line Mendelian Inheritance in Man database found at the world wide website of the National Center of Biotechnology Information (NCBI). Additional description of native GPCRs may be found at www.primalinc.com. A native GPCR is a target for drug therapy if the GPCR is implicated in a GPCR-related disorder.

A “known GPCR” is a native GPCR for which the native ligand specific for that GPCR has been identified.

An “orphan GPCR” is a native GPCR for which the native ligand specific for that GPCR has not been identified or is not known.

A “liganded-orphan GPCR” is an orphan GPCR for which a non-native ligand specific for that GPCR has been identified.

A “parental GPCR” is a native GPCR or a altered native GPCR (e.g. a native GPCR that is, for example operably linked to another protein, or contains substitutions, insertions or deletions of amino acids such that the GPCR has the same activity, e.g., ligand-binding, as a parental GPCR). A parental GPCR may be substituted with a “GPCR-upregulating cassette” amino acid sequence within its TM5-IC3-TM6 segment so as to become a substituted GPCR. A “substituted GPCR” may be a ligand upregulatable GPCR if its detectable amount in or on a cell increases upon binding of a ligand to the substituted GPCR.

As such, a parental GPCR, a substituted GPCR, and a ligand upregulatable GPCR are all related and derived at their sequence level from a native GPCR.

A “TM5-IC3-TM6” segment of a GPCR is a segment of a GPCR that is defined by the TM5, IC3 and TM6 regions, as further discussed below. When a TM5-IC3-TM6 segment is modified, a portion of the segment is modified.

A “GCPR-upregulating cassette” or “GURC” may be used to modify a parental GPCR to make it into a ligand upregulatable GPCR. The word “upregulating” in this term is used solely as a matter of convenience and is not meant to indicate a mechanism by which a GPCR becomes ligand upregulatable.

The term “ligand” means a molecule that specifically binds to a GPCR. A ligand may be, for example a polypeptide, a lipid, a small molecule, an antibody. A “native ligand” is a ligand that is an endogenous, natural ligand for a native GPCR. A ligand may be a GPCR “antagonist”, “agonist”, “partial agonist” or “inverse agonist”, or the like.

A “modulator” is a ligand that increases or decreases a GPCR intracellular response when it is in contact with, e.g., binds, to a GPCR that is expressed in a cell.

An “agonist” is a ligand which activates a GPCR intracellular response when it binds to a GPCR

A “partial agonist” is a ligand what activates, to a lesser extent than an agonist, a GPCR intracellular response when it binds to a GPCR.

An “antagonist” is a ligand which competitively binds to a GPCR at the same site as an agonist but which does not activate the intracellular response produced by the active form of a GPCR. Antagonists usually inhibit intracellular responses by an agonist or partial agonist. Antagonists usually do not diminish the baseline intracellular response in the absence of an agonist or partial agonist.

An “inverse agonist” is a ligand which binds to a GPCR and inhibits the baseline intracellular response of the GPCR observed in the absence of an agonist or partial agonist. In most embodiments, a baseline intracellular response is inhibited in the presence of an inverse agonist by at least about 30%, by at least about 50%, or by at least 75%, as compared to a baseline response in the absence of an inverse agonist.

As used herein, the terms “GPCR-related condition” and “GPCR-related disorder” are used interchangeably to refer to any disorder, or symptoms of which, caused by or treatable by an alteration in the activity of a specific GPCR. GPCR-related conditions may be associated with aberrant activity of a GPCR, and may be caused by aberrant activity of a GPCR, such as in the cases where a GPCR is mutated to cause an over-active, constitutively active, or under-active GPCR Also within this definition are disorders treatable by altering the activity of a GPCR that has normal activity. For example, some disorders are not associated with the aberrant activity of a particular GPCR, but nevertheless are treatable by modulating that GPCR. Also encompassed by this term are cosmetic alterations, which are not life threatening but otherwise desirable to have. Exemplary GPCR-related conditions include allergies, hypertension, psychological disorders e.g. depression, anxiety and schizophrenia, migraine headaches, reflux, asthma and bronchospasm, prostatic hypertrophy, ulcers, epilepsy, angina, rhinitis, cancer e.g. prostate cancer, glaucoma and stroke. Further exemplary GPCR-related conditions at the On-line Mendelian Inheritance in Man database found at the world wide website of the NCBI.

The term “phenomenon associated with aberrant GPCR activity” as used herein refers to a structural, molecular, or functional characteristic associated with aberrant GPCR activity, particularly such a characteristic that is readily assessable in a human or animal model. Such characteristics include, but are not limited to, downstream molecular events caused by activation of a GPCR, and phenotypes or symptoms, for example, sneezing, nasal mucous production, acid reflux, mood, wheezing, pain, height, etc.

A “deletion” is defined as a change in either amino acid or nucleotide sequence in which one or more amino acid or nucleotide residues, respectively, are absent as compared to an amino acid sequence or nucleotide sequence of a parental GPCR polypeptide or nucleic acid. In the context of a GPCR or a fragment thereof, a deletion can involve deletion of about 2, about 5, about 10, up to about 20, up to about 30 or up to about 50 or more amino acids. A GPCR or a fragment thereof may contain more than one deletion.

An “insertion” or “addition” is that change in an amino acid or nucleotide sequence which has resulted in the addition of one or more amino acid or nucleotide residues, respectively, as compared to an amino acid sequence or nucleotide sequence of a parental GPCR. “Insertion” generally refers to addition to one or more amino acid residues within an amino acid sequence of a polypeptide, while “addition” can be an insertion or refer to amino acid residues added at an N- or C-terminus, or both termini. In the context of a GPCR or fragment thereof, an insertion or addition is usually of about 1, about 3, about 5, about 10, up to about 20, up to about 30 or up to about 50 or more amino acids. A GPCR or fragment thereof may contain more than one insertion.

A “substitution” results from the replacement of one or more amino acids or nucleotides by different amino acids or nucleotides, respectively as compared to an amino acid sequence or nucleotide sequence of a parental GPCR or a fragement thereof. It is understood that a GPCR or a fragment thereof may have conservative amino acid substitutions which have substantially no effect on GPCR activity. By conservative substitutions is intended combinations such as gly, ala; val, ile, leu; asp, glu; asn, gln; ser, thr; lys, arg; and phe, tyr.

The term “biologically active” GPCR refers to a GPCR having structural and biochemical functions of a naturally occurring GPCR.

As used herein, the terms “determining,” “measuring,” “assessing,” and “assaying” are used interchangeably and include both quantitative and qualitative determinations. Reference to an “amount” of a GPCR in these contexts is not intended to require quantitative assessment, and may be either qualitative or quantitative, unless specifically indicated otherwise.

The terms “polypeptide” and “protein”, used interchangeably herein, refer to a polymeric form of amino acids of any length, which can include coded and non-coded amino acids, chemically or biochemically modified or derivatized amino acids, and polypeptides having modified peptide backbones. The term includes fusion proteins, including, but not limited to, fusion proteins with a heterologous amino acid sequence, fusions with heterologous and homologous leader sequences, with or without N-terminal methionine residues; immunologically tagged proteins; fusion proteins with detectable fusion partners, e.g., fusion proteins including as a fusion partner a fluorescent protein, β-galactosidase, luciferase, etc.; and the like.

The terms “nucleic acid molecule” and “polynucleotide” are used interchangeably and refer to a polymeric form of nucleotides of any length, either deoxyribonucleotides or ribonucleotides, or analogs thereof. Polynucleotides may have any three-dimensional structure, and may perform any function, known or unknown. Non-limiting examples of polynucleotides include a gene, a gene fragment, exons, introns, messenger RNA (mRNA), transfer RNA, ribosomal RNA, ribozymes, cDNA, recombinant polynucleotides, branched polynucleotides, plasmids, vectors, isolated DNA of any sequence, control regions, isolated RNA of any sequence, nucleic acid probes, and primers. The nucleic acid molecule may be linear or circular.

As used herein the term “isolated,” when used in the context of an isolated compound, refers to a compound of interest that is in an environment different from that in which the compound naturally occurs. “Isolated” is meant to include compounds that are within samples that are substantially enriched for the compound of interest and/or in which the compound of interest is partially or substantially purified.

As used herein, the term “substantially pure” refers to a compound that is removed from its natural environment and is at least 60% free, preferably 75% free, and most preferably 90% free from other components with which it is naturally associated.

A “coding sequence” or a sequence that “encodes” a selected polypeptide, is a nucleic acid molecule which can be transcribed (in the case of DNA) and translated (in the case of MRNA) into a polypeptide , for example, in a host cell when placed under the control of appropriate regulatory sequences (or “control elements”). The boundaries of the coding sequence are typically determined by a start codon at the 5′ (amino) terminus and a translation stop codon at the 3′ (carboxy) terminus. A coding sequence can include, but is not limited to, cDNA from viral, procaryotic or eucaryotic MRNA, genomic DNA sequences from viral or prokaryotic DNA, and synthetic DNA sequences. A transcription termination sequence may be located 3′ to the coding sequence. Other “control elements” may also be associated with a coding sequence. A DNA sequence encoding a polypeptide can be optimized for expression in a selected cell by using the codons preferred by the selected cell to represent the DNA copy of the desired polypeptide coding sequence.

“Encoded by” refers to a nucleic acid sequence which codes for a polypeptide sequence, wherein the polypeptide sequence or a portion thereof contains an amino acid sequence of at least 3 to 5 amino acids, more preferably at least 8 to 10 amino acids, and even more preferably at least 15 to 20 amino acids from a polypeptide encoded by the nucleic acid sequence. Also encompassed are polypeptide sequences that are immunologically identifiable with a polypeptide encoded by the sequence.

“Operably linked” refers to an arrangement of elements wherein the components so described are configured so as to perform their usual function. Thus, a given GPCR that is operably linked to a GURC is a GPCR that contains a TM5-IC3-TM6 segment that is substituted by a ligand upregulatability providing amino acid sequence, i.e., a GURC, as described above. In the case of a promoter, a promoter that is operably linked to a coding sequence will effect the expression of a coding sequence. The promoter or other control elements need not be contiguous with the coding sequence, so long as they function to direct the expression thereof. For example, intervening untranslated yet transcribed sequences can be present between the promoter sequence and the coding sequence and the promoter sequence can still be considered “operably linked” to the coding sequence.

By “nucleic acid construct” it is meant a nucleic acid sequence that has been constructed to comprise one or more functional units not found together in nature. Examples include circular, linear, double-stranded, extrachromosomal DNA molecules (plasmids), cosmids (plasmids containing COS sequences from lambda phage), viral genomes comprising non-native nucleic acid sequences, and the like.

A “vector” is capable of transferring gene sequences to a host cell. Typically, “vector construct,” “expression vector,” and “gene transfer vector,” mean any nucleic acid construct capable of directing the expression of a gene of interest and which can transfer gene sequences to host cells, which can be accomplished by genomic integration of all or a portion of the vector, or transient or inheritable maintenance of the vector as an extrachromosomal element. Thus, the term includes cloning, and expression vehicles, as well as integrating vectors.

An “expression cassette” comprises any nucleic acid construct capable of directing the expression of a gene/coding sequence of interest, which is operably linked to a promoter of the expression cassette. Such cassettes can be constructed into a “vector,” “vector construct,” “expression vector,” or “gene transfer vector,” in order to transfer the expression cassette into a host cell. Thus, the term includes cloning and expression vehicles, as well as viral vectors.

Techniques for determining nucleic acid and amino acid “sequence identity” are known in the art. Typically, such techniques include determining the nucleotide sequence of the MRNA for a gene and/or determining the amino acid sequence encoded thereby, and comparing these sequences to a second nucleotide or amino acid sequence. In general, “identity” refers to an exact nucleotide-to-nucleotide or amino acid-to-amino acid correspondence of two polynucleotides or polypeptide sequences, respectively. Two or more sequences (polynucleotide or amino acid) can be compared by determining their “percent identity.” The percent identity of two sequences, whether nucleic acid or amino acid sequences, is the number of exact matches between two aligned sequences divided by the length of the shorter sequences and multiplied by 100.

An approximate alignment for nucleic acid sequences is provided by the local homology algorithm of Smith and Waterman, Advances in Applied Mathematics 2:482-489 (1981). This algorithm can be applied to amino acid sequences by using the scoring matrix developed by Dayhoff, Atlas of Protein Sequences and Structure, M. O. Dayhoff ed., 5 suppl. 3:353-358, National Biomedical Research Foundation, Washington, D.C., USA, and normalized by Gribskov, Nucl. Acids Res. 14(6):6745-6763 (1986). An exemplary implementation of this algorithm to determine percent identity of a sequence is provided by the Genetics Computer Group (Madison, Wis.) in the “BestFit” utility application. The default parameters for this method are described in the Wisconsin Sequence Analysis Package Program Manual, Version 8 (1995) (available from Genetics Computer Group, Madison, Wis.). A preferred method of establishing percent identity in the context of the present invention is to use the MPSRCH package of programs copyrighted by the University of Edinburgh, developed by John F. Collins and Shane S. Sturrok, and distributed by IntelliGenetics, Inc. (Mountain View, Calif.). From this suite of packages the Smith-Waterman algorithm can be employed where default parameters are used for the scoring table (for example, gap open penalty of 12, gap extension penalty of one, and a gap of six). From the data generated the “Match” value reflects “sequence identity.” Other suitable programs for calculating the percent identity or similarity between sequences are generally known in the art, for example, another alignment program is BLAST, used with default parameters. For example, BLASTN and BLASTP can be used using the following default parameters: genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+Swiss protein+Spupdate+PIR. Details of these programs can be found at the following internet address: http://www.ncbi.nlm.gov/cgi-bin/BLAST.

Alternatively, sequence identity can be determined by hybridization of polynucleotides under conditions that form stable duplexes between identical regions, followed by digestion with single-stranded-specific nuclease(s), and size determination of the digested fragments. Two DNA, or two polypeptide sequences are “substantially identical” to each other when the sequences exhibit at least about 80%-85%, preferably at least about 85%-90%, more preferably at least about 90%-95%, and most preferably at least about 95%-98% sequence identity over a defined length of the molecules, as determined using the methods above. As used herein, substantially identical also refers to sequences showing complete identity to the specified DNA or polypeptide sequence. DNA sequences that are substantially identical can be identified in a Southern hybridization experiment under, for example, stringent conditions, as defined for that particular system. Defining appropriate hybridization conditions is within the skill of the art. See, e.g., Sambrook et al., infra; DNA Cloning, supra; Nucleic Acid Hybridization, supra.

Two nucleic acid fragments are considered to “selectively hybridize” as described herein. The degree of sequence identity between two nucleic acid molecules affects the efficiency and strength of hybridization events between such molecules. A partially identical nucleic acid sequence will at least partially inhibit a completely identical sequence from hybridizing to a target molecule. Inhibition of hybridization of the completely identical sequence can be assessed using hybridization assays that are well known in the art (e.g., Southern blot, Northern blot, solution hybridization, or the like, see Sambrook, et al., Molecular Cloning: A Laboratory Manual, Second Edition, (1989) Cold Spring Harbor, N.Y.). Such assays can be conducted using varying degrees of selectivity, for example, using conditions varying from low to high stringency. If conditions of low stringency are employed, the absence of non-specific binding can be assessed using a secondary probe that lacks even a partial degree of sequence identity (for example, a probe having less than about 30% sequence identity with the target molecule), such that, in the absence of non-specific binding events, the secondary probe will not hybridize to the target.

When utilizing a hybridization-based detection system, a nucleic acid probe is chosen that is complementary to a target nucleic acid sequence, and then by selection of appropriate conditions the probe and the target sequence “selectively hybridize,” or bind, to each other to form a hybrid molecule. A nucleic acid molecule that is capable of hybridizing selectively to a target sequence under “moderately stringent” typically hybridizes under conditions that allow detection of a target nucleic acid sequence of at least about 10-14 nucleotides in length having at least approximately 70% sequence identity with the sequence of the selected nucleic acid probe. Stringent hybridization conditions typically allow detection of target nucleic acid sequences of at least about 10-14 nucleotides in length having a sequence identity of greater than about 90-95% with the sequence of the selected nucleic acid probe. Hybridization conditions useful for probe/target hybridization where the probe and target have a specific degree of sequence identity, can be determined as is known in the art (see, for example, Nucleic Acid Hybridization: A Practical Approach, editors B. D. Hames and S. J. Higgins, (1985) Oxford; Washington, D.C.; IRL Press).

With respect to stringency conditions for hybridization, it is well known in the art that numerous equivalent conditions can be employed to establish a particular stringency by varying, for example, the following factors: the length and nature of probe and target sequences, base composition of the various sequences, concentrations of salts and other hybridization solution components, the presence or absence of blocking agents in the hybridization solutions (e.g., formamide, dextran sulfate, and polyethylene glycol), hybridization reaction temperature and time parameters, as well as, varying wash conditions. The selection of a particular set of hybridization conditions is selected following standard methods in the art (see, for example, Sambrook, et al., Molecular Cloning: A Laboratory Manual, Second Edition, (1989) Cold Spring Harbor, N.Y.). An example of stringent hybridization conditions is hybridization at 50° C. or higher and 0.1×SSC (15 mM sodium chloride/1.5 mM sodium citrate). Another example of stringent hybridization conditions is overnight incubation at 42° C. in a solution: 50% formamide, 5×SSC (150 mM NaCl, 15 mM trisodium citrate), 50 mM sodium phosphate (pH 7.6), 5×Denhardt's solution, 10% dextran sulfate, and 20 mg/ml denatured, sheared salmon sperm DNA, followed by washing the filters in 0.1×SSC at about 65° C. Stringent hybridization conditions are hybridization conditions that are at least as stringent as the above representative conditions, where conditions are considered to be at least as stringent if they are at least about 80% as stringent, typically at least about 90% as stringent as the above specific stringent conditions. Other stringent hybridization conditions are known in the art and may also be employed to identify nucleic acids of this particular embodiment of the invention.

A first polynucleotide is “derived from” or “corresponds to” a second polynucleotide if it has the same or substantially the same nucleotide sequence as a region of the second polynucleotide, its cDNA, complements thereof, or if it displays sequence identity as described above.

A first polypeptide is “derived from” or “corresponds to” a second polypeptide if it is (i) encoded by a first polynucleotide derived from a second polynucleotide, or (ii) displays sequence identity to the second polypeptides as described above.

The terms “treatment”, “treating”, “treat”, and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse affect attributable to the disease. “Treatment”, as used herein, covers any treatment of a disease in a mammal, particularly in a human, and includes: (a) preventing the disease from occurring in a subject which may be predisposed to the disease but has not yet been diagnosed as having it; (b) inhibiting the disease, i.e., arresting its development; and (c) relieving the disease, i.e., causing regression of the disease and/or relieving one or more disease symptoms. “Treatment” is also meant to encompass delivery of an agent in order to provide for a pharmacologic effect, even in the absence of a disease or condition. For example, “treatment” encompasses delivery of GPCR-modulator that can provide for enhanced or desirable effects in the subject (e.g., reduction of pathogen load, beneficial increase in a physiological parameter of the subject, reduction of disease symptoms, etc.).

“Subject”, “individual,” “host” and “patient” are used interchangeably herein, to refer to an animal, human or non-human, susceptible to or having a GPCR-related disorder amenable to therapy according to the methods of the invention. Generally, the subject is a mammalian subject. Exemplary subjects include, but are not necessarily limited to, humans, non-human primates, mice, rats, cattle, sheep, goats, pigs, dogs, cats, and horses, with humans being of particular interest.

### DETAILED DESCRIPTION OF THE INVENTION

The subject invention provides methods for making ligand upregulatable G-protein coupled receptors (GPCRs). Ligand upregulatable GPCRs contain a modified TM5-IC3-TM6 segment, which provides for increased levels of the ligand upregulatable GPCR in a host cell in the presence of a ligand as compared to the absence of the ligand. The subject invention also provides assays for screening test compounds for a ligand of a GPCR using a ligand upregulatable GPCR. In these assays, a ligand upregulatable GPCR is contacted with a test compound, and an increase in the detectable amount of the ligand upregulatable GPCR in the host cell indicates that the candidate agent is a ligand of the GPCR Said indicated ligands encompass modulators of the GPCR. Said modulators of the GPCR are particularly useful in treating GPCR-related conditions.

In further describing the invention in greater detail than provided in the Summary and as informed by the Background and Definitions provided above, methods of making a ligand upregulatable GPCR are described first, followed by a discussion of methods of determining whether a test compound is a ligand of a GPCR using a ligand upregulatable GPCR. This discussion is followed by a description of a review of representative applications in which the subject methods find use, as well as the subject kits provided for practicing the subject methods.

Methods for Making a Ligand Upregulatable GPCR

In one aspect, the invention provides methods of making a ligand upregulatable GPCR. In general, the ligand upregulatable GPCR is a modified version of naturally occurring GPCR or a biologically active variant thereof, that contains a modified TM5-IC3-TM6 segment. In most embodiments, the modified TM5-IC3-TM6 segment is provided by a GPCR-upregulating cassette. In most embodiments, a ligand upregulatable GPCR is detectably present in or on a cell in greater amounts in the presence of a ligand, as compared to the same ligand upregulatable GPCR in the absence of the ligand.

In general, the subject methods involve identifying the TM5-IC3-TM6 segment corresponding to the TM5, IC3, and TM6 regions of a parental GPCR and substituting at least part of the identified segment with the amino acid sequence of a GPCR upregulating cassette to make a substituted GPCR Substituted GPCRs are usually expressed in a host cell, and tested for upregulation by ligand by comparing a detectable amount of the substituted GPCR in the presence of a ligand to a detectable amount of the substituted GPCR in the absence of the ligand. As one of skill in the art would recognize, the determination of the detectable amount of a substituted GPCR may be performed in parallel or in series with the control experiments (e.g. by determining the relative amounts of GPCR before and after addition of a ligand). A substituted GPCR for which expression is upregulated in the presence of a ligand as compared to the absence of the ligand is a ligand upregulatable GPCR.

The detectable amount of a substituted GPCR may be determined by a number of methods, including by its detection by a binding partner, e.g., an antibody. In many embodiments however, in order to facilitate making a determination of the detectable amount of a substituted GPCR, the substituted GPCR is fused to a reporter polypeptide, such as GFP, luciferase, or the like. One suitable reporter polypeptide is Renilla luciferase. As such, the detectable amount of a substituted GPCR is usually determined by measuring reporter activity, and a reporter activity that is higher than a control reporter activity indicates that the substituted GPCR is a ligand upregulatable GPCR. In certain embodiments the reporter polypeptide is wild type or modified Renilla luciferase.

In the following description, GPCRs are described first, followed by a discussion of the GPCR TM5, IC3, and TM6 regions. Following this discussion is a description of a GPCR upregulating cassette and methods by which a skilled person may make a ligand upregulatable GPCR.

G-Protein Coupled Receptors

Any known GPCR is suitable for use in the subject methods, as long as it has TM5, IC3, and TM6 regions that are identifiable in the sequence of the GPCR. A disclosure of the sequences and phylogenetic relationships between 277 GPCRs is provided in Joost et al. (Genome Biol. 2002 3:RESEARCH0063, the entire contents of which is incorporated by reference) and, as such, at least 277 GPCRs are suitable for the subject methods. A more recent disclosure of the sequences and phylogenetic relationships between 367 human and 392 mouse GPCRs is provided in Vassilatis et al. (Proc Natl Acad Sci 2003 100:4903-8 and www.primalinc.com, each of which is hereby incorporated by reference in its entirely) and, as such, at least 367 human and at least 392 mouse GPCRs are suitable for the subject methods.

The methods may be used, by way of exemplification, for purinergic receptors, vitamin receptors, lipid receptors, peptide hormone receptors, non-hormone peptide receptors, non-peptide hormone receptors, polypeptide receptors, protease receptors, receptors for sensory signal mediator, and biogenic amine receptors not including β2-adrenergic receptor. In certain embodiments, said biogenic amine receptor does not include an adrenoreceptor. α-type adrenoreceptors (e.g. α_{1A}, α_{1B} or α_{1C} adrenoreceptors), and β-type adrenoreceptors (e.g. β₁, β₂, or β₃ adrenoreceptors) are discussed in Singh et al., J. Cell Phys. 189:257-265, 2001.

It is recognized that both native and altered native GPCRs may be used in the subject methods. In certain embodiments, therefore, an altered native GPCR (e.g. a native GPCR that is altered by addition such as an addition of a reporter, substitution, deletions and insertions) such that it binds the same ligand as a corresponding native GPCR. In the subject methods a “parental” GPCR may be a native GPCR or an altered native GPCR.

As such, the following GPCRs (native or altered) find particular use as parental GPCRs in the subject methods: cholinergic receptor, muscarinic 3; melanin-concentrating hormone receptor 2; cholinergic receptor, muscarinic 4; niacin receptor; histamine 4 receptor; ghrelin receptor; CXCR3 chemokine receptor; motilin receptor; 5-hydroxytryptamine (serotonin) receptor 2A; 5-hydroxytryptamine (serotonin) receptor 2B; 5-hydroxytryptamine (serotonin) receptor 2C; dopamine receptor D3; dopamine receptor D4; dopamine receptor D1; histamine receptor H2; histamine receptor H3; galanin receptor 1; neuropeptide Y receptor Y1; angiotensin II receptor 1; neurotensin receptor 1; melanocortin 4 receptor; glucagon-like peptide 1 receptor; adenosine A1 receptor; cannabinoid receptor 1; melanin-concentrating hormone receptor 1; GPR40; and GPCR2.

GPCR TM5, IC-3, and TM6 Regions

In the subject methods, the TM5-IC3-TM6 segment corresponding to the TM5, IC-3, and TM6 regions of a parental GPCR is usually identified and modified such that it comprises the amino acid sequence of a GPCR upregulating cassette. As such, in most embodiments, the subject methods involve identifying the TM5, IC-3, and TM6 regions of a GPCR. The schematic representation of the prototypical structure of a GPCR is provided in FIG. 1, where these regions, in the context of the entire structure of a GPCR, may be seen.

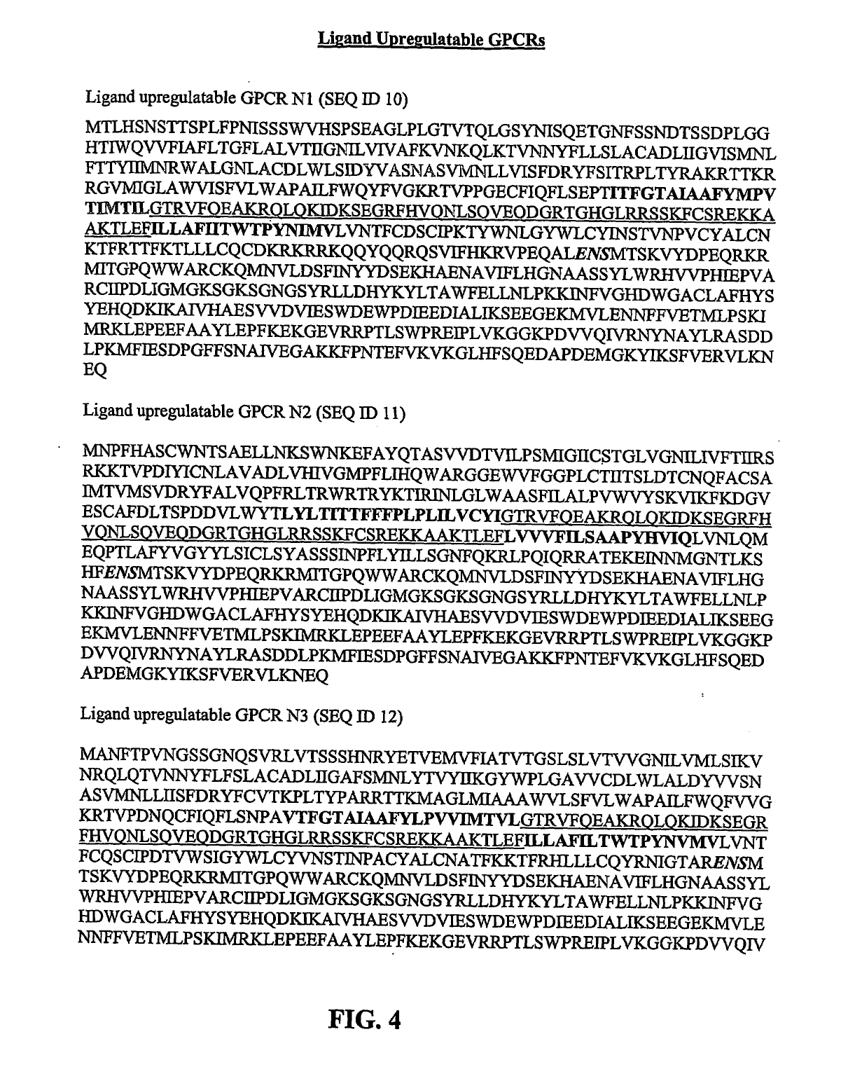

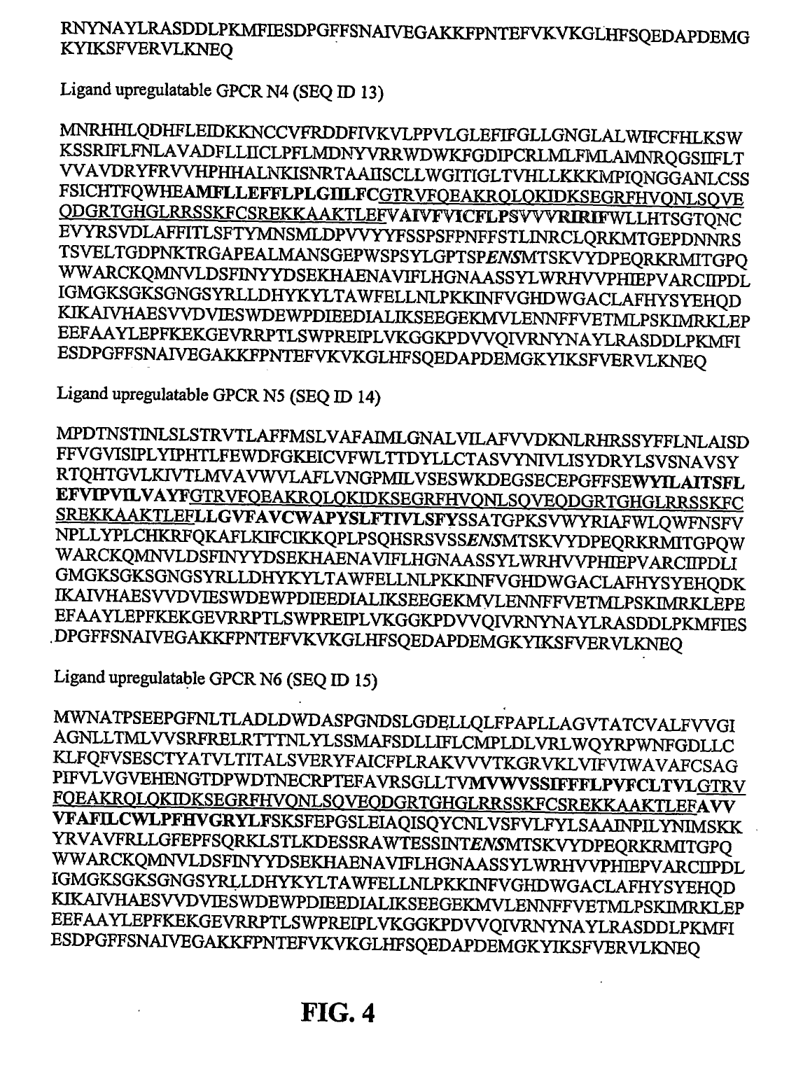

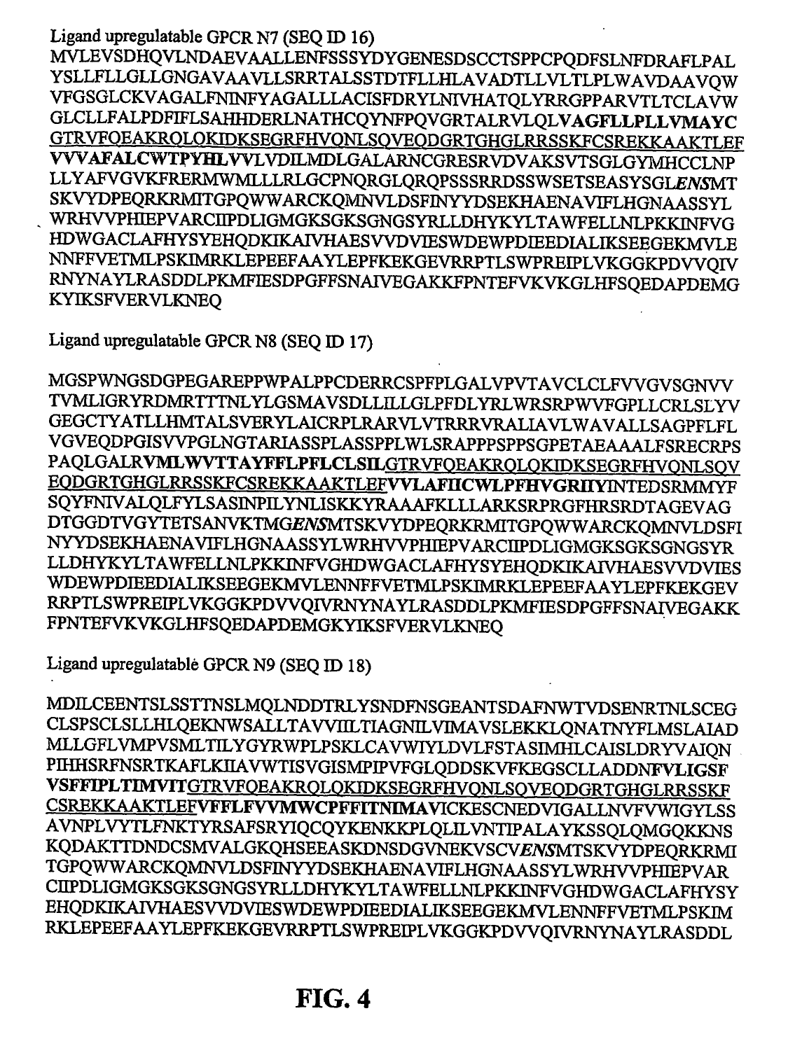

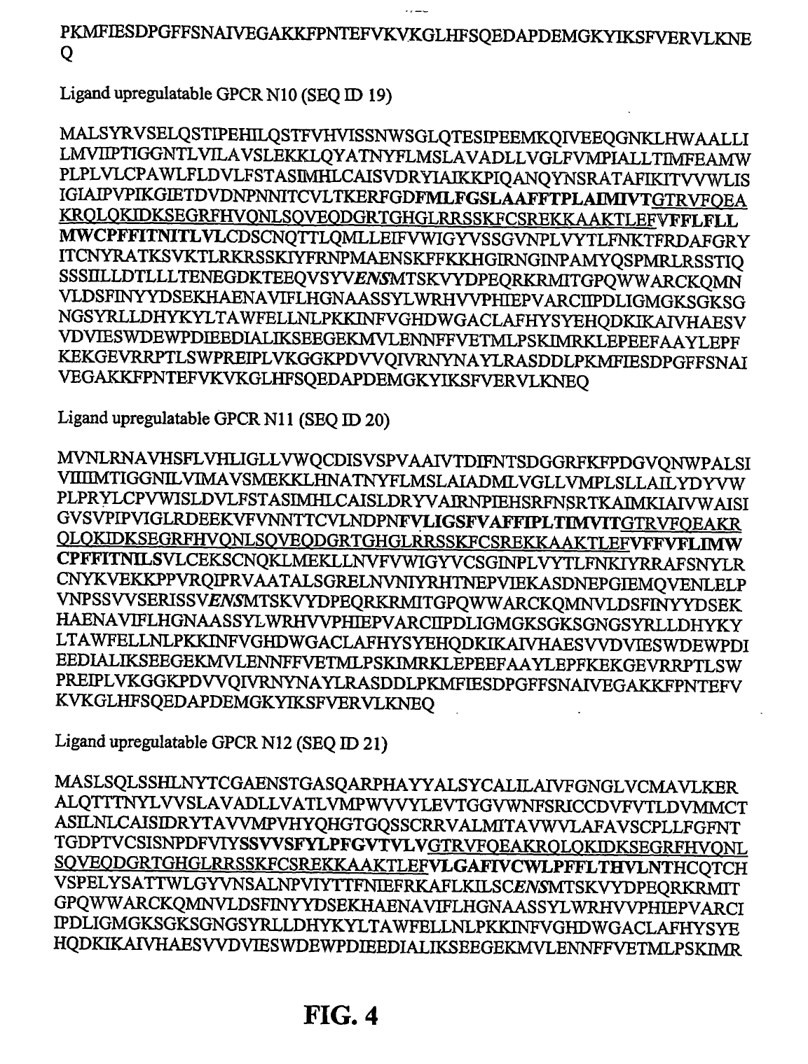

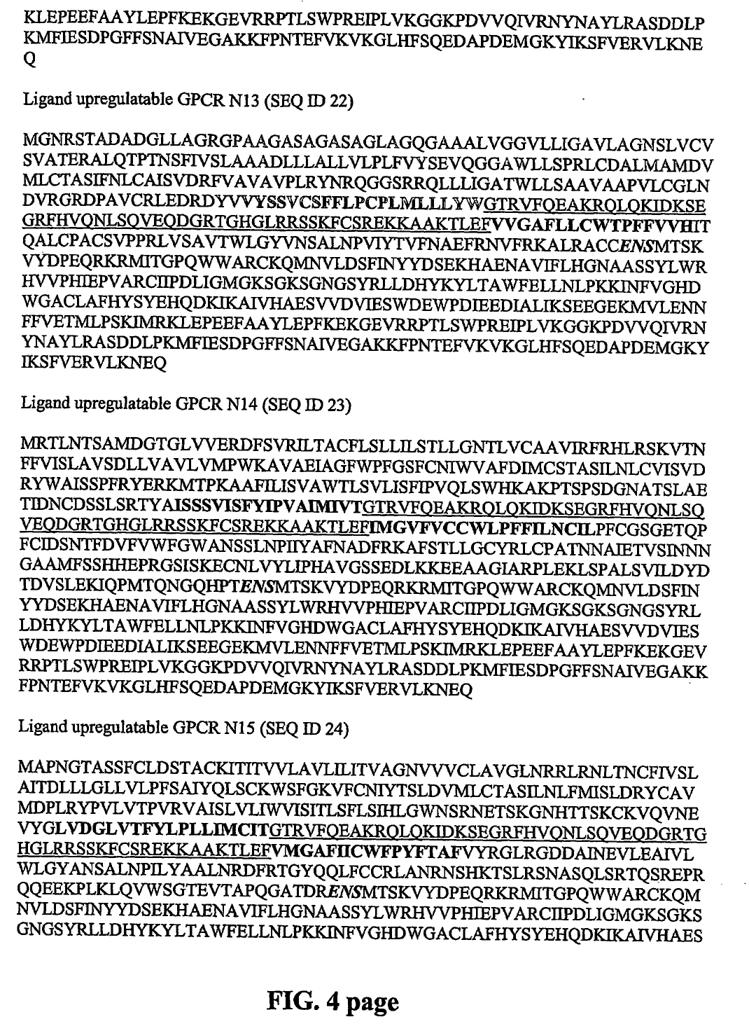

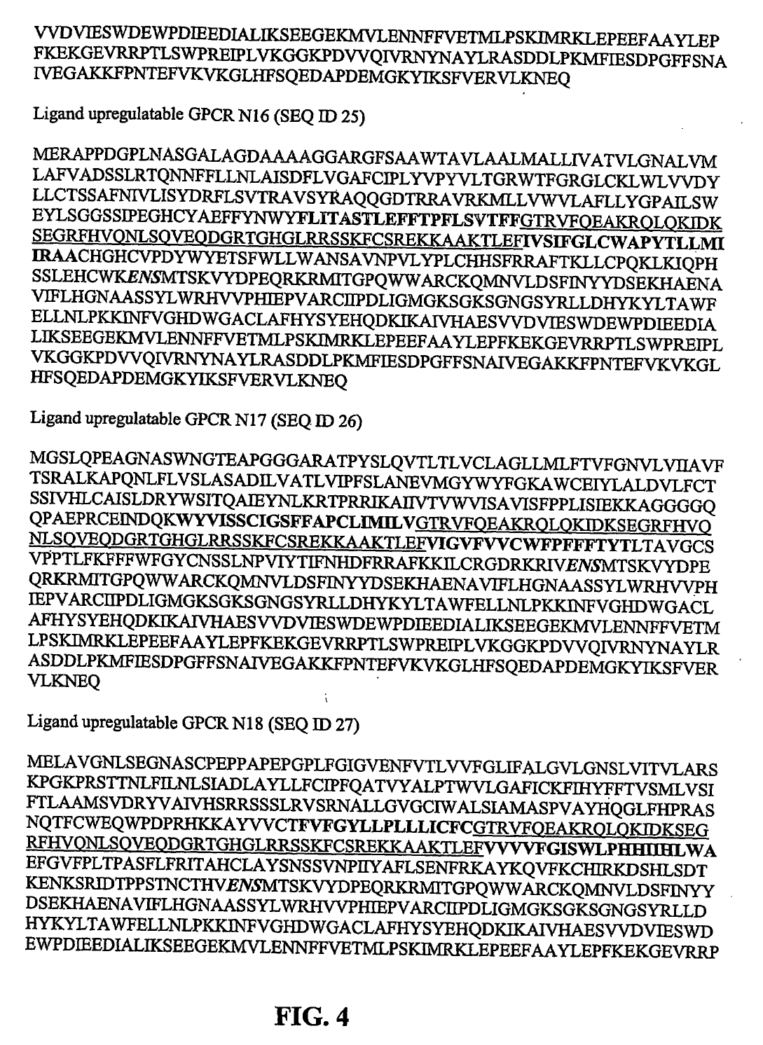

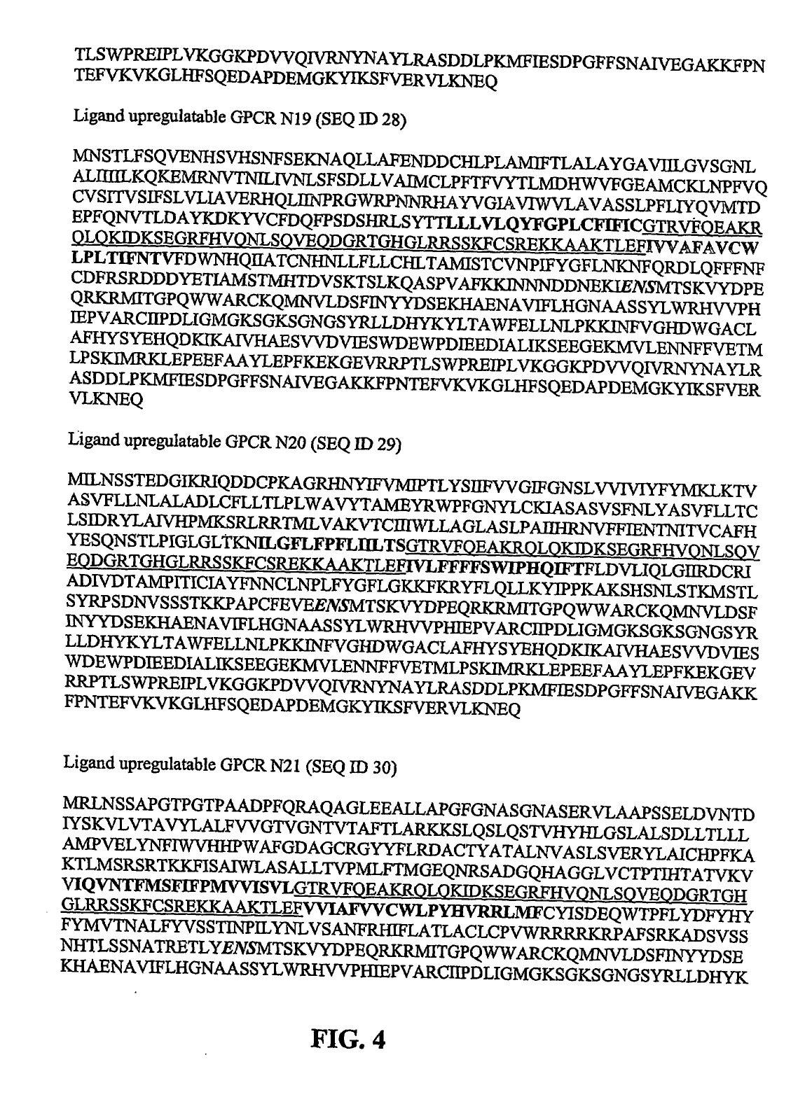

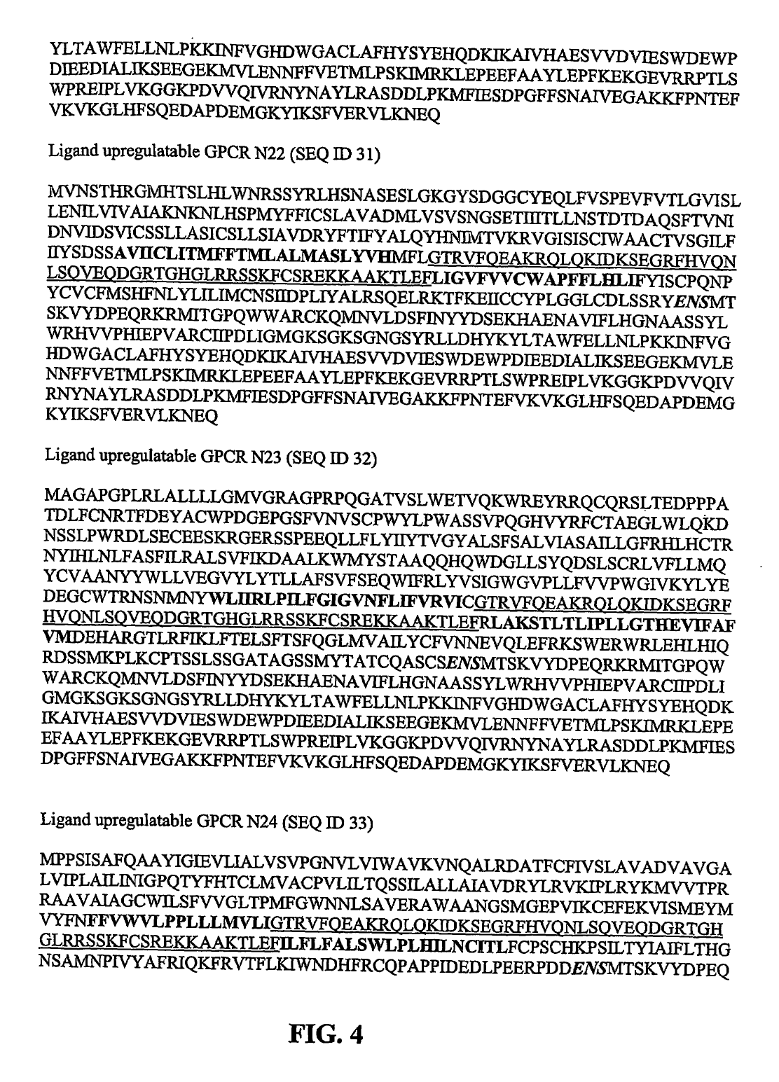

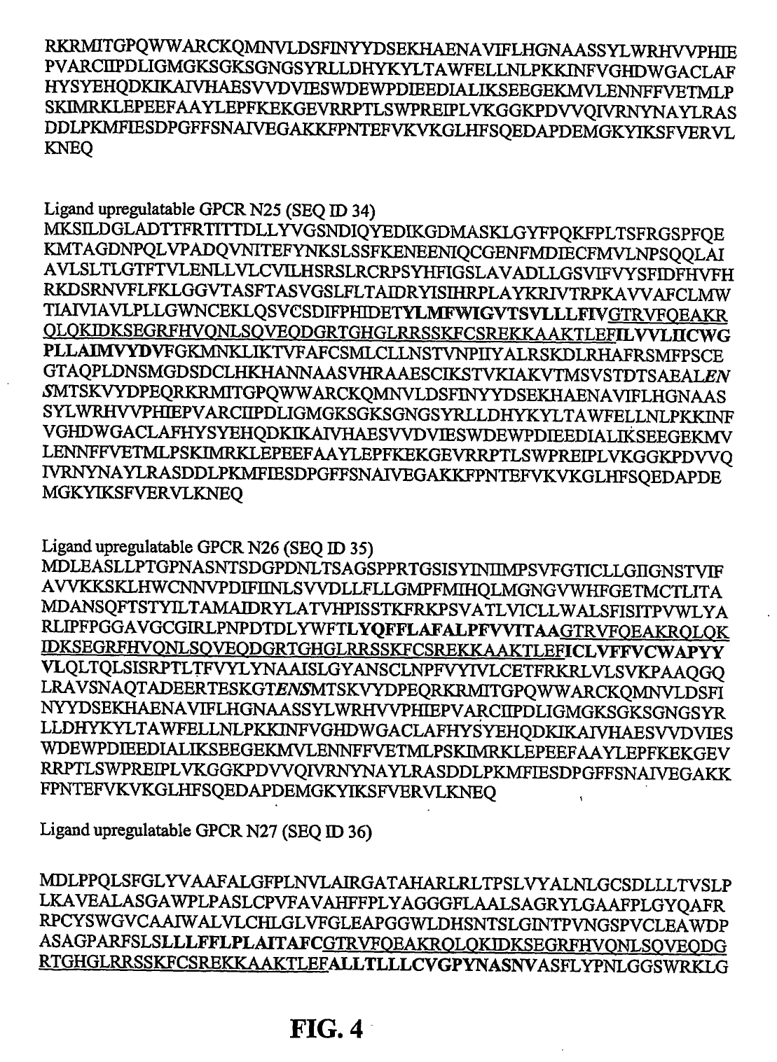

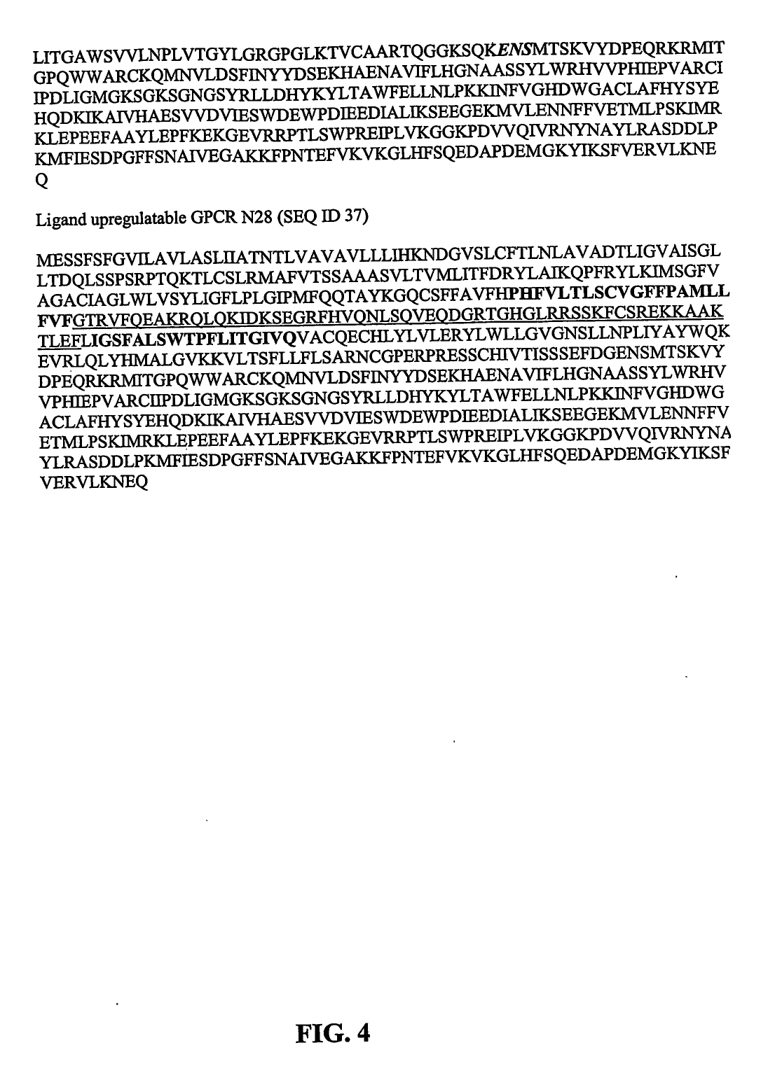

The IC3 region of a GPCR lies in between transmembrane regions TM5 and TM6 and, may usually be about 12 amino acids (CXCR3 and GPR40) to about 235 amino acids (cholinergic receptor, muscarinic 3) in lengths. The TM5, IC3, and TM6 regions are readily discernable by one of skill in the art using, for example, a program for identifying transmembrane regions; once transmembrane regions TM5 and TM6 regions are identified, the IC3 region will be apparent. The TM5, IC3, and TM6 regions may also be identified using such methods as pairwise or multiple sequence alignment (e.g. using the GAP or BESTFIT of the University of Wisconsin's GCG program, or CLUSTAL alignment programs, Higgins et al., Gene. 1988 73:237-44), using a target GPCR and, for example, GPCRs of known structure, or the upregulatable GPCRs shown in FIG. 4.

Suitable programs for identifying transmembrane regions include those described by Moller et al., (Bioinformatics, 17:646-653, 2001). A particularly suitable program is called “TMHMM” Krogh et al., (Journal of Molecular Biology, 305:567-580,2001). To use these programs via a user interface, a sequence corresponding to a GPCR or a fragment thereof is entered into the user interface and the program run. Such programs are currently available over the world wide web, for example at the website of the Center for Biological Sequence Analysis at cbs.dtu.dk/services/. The output of these programs may be variable in terms its format, however they usually indicate transmembrane regions of a GPCR using amino acid coordinates of a GPCR. For example, if the contiguous 413 amino acids of the human adrenergic β2 receptor are analyzed by TMHMM at default settings, the output is as follows, where a transmembrane domain “TM” is indicated by “TMhelix”, beginning (beg) and ending (end) at the amino acid positions indicated.

# Sequence Length: 413
# Sequence Number of predicted TMHs: 7
# Sequence Exp number of AAs in TMHs: 148.9586
# Sequence Exp number, first 60 AAs: 22.976
# Sequence Total prob of N-in: 0.00400
# Sequence POSSIBLE N-term signal sequence

| | | TM | beg | end |
|---|---|---|---|---|
| Sequence | TMHMM2.0 | outside | 1 | 35 |
| Sequence | TMHMM2.0 | TMhelix | 36 | 58 |
| Sequence | TMHMM2.0 | inside | 59 | 69 |
| Sequence | TMHMM2.0 | TMhelix | 70 | 92 |
| Sequence | TMHMM2.0 | outside | 93 | 106 |
| Sequence | TMHMM2.0 | TMhelix | 107 | 129 |
| Sequence | TMHMM2.0 | inside | 130 | 149 |
| Sequence | TMHMM2.0 | TMhelix | 150 | 169 |
| Sequence | TMHMM2.0 | outside | 170 | 200 |
| Sequence | TMHMM2.0 | TMhelix | 201 | 223 |
| Sequence | TMHMM2.0 | inside | 224 | 274 |
| Sequence | TMHMM2.0 | TMhelix | 275 | 297 |
| Sequence | TMHMM2.0 | outside | 298 | 306 |
| Sequence | TMHMM2.0 | TMhelix | 307 | 326 |
| Sequence | TMHMM2.0 | inside | 327 | 413 |

As such, one of skill in the art would recognize that the TM5 region for this GPCR corresponds to the contiguous amino acids of positions 201-223, the IC3 region for this GPCR corresponds to the contiguous amino acids of positions 224-274 of this GPCR, and the TM6 region for this GPCR corresponds to contiguous amino acids of positions 275-297. The TM5-IC3-TM6 segment is understood to correspond to the contiguous amino acids of positions 201-297. All GPCRs can be analyzed using the same program to give a suitable prediction of the amino acid coordinates of the TM5, IC3, and TM6 regions of the GPCR.

When TM regions of a GPCR polypeptide are determined using TMHMM, the prototypical GPCR profile is usually obtained: an N-terminus that is extracellular, followed by a segment comprising seven TM regions, and further followed by a C-terminus that is intracellular. TM numbering for this prototypical GPCR profile begins with the most N-terminally disposed TM region (TM1) and concludes with the most C-terminally disposed TM region (TM7).

On occasion, TMHMM provides either eight or six TM regions for a GPCR polypeptide. In this situation, the N-terminus is taken to be fixed as extracellular and the C-terminus is taken to be fixed as intracellular, and the TM numbering is carried out as follows. A) If TMHMM provides eight TM regions and an intracellular N-terminus, e.g., as is the case for hydroxytryptamine (serotonin) receptor 2C, then the most N-terminally disposed TM region is ignored and numbering begins with the second TM region (TM1) and concludes with the most C-terminally disposed TM region (TM7). B) If TMHMM provides eight TM regions and an extracellular C-terminus, then the most C-terminally disposed TM region is ignored and numbering begins with the most N-terminally disposed TM region (TM1) and concludes with the seventh TM region (TM7). C) If TMHMM provides six TM regions (as is the case for neurotensin receptor 1), then the internal TM region rejected by TMHMM having greatest probability is accepted, and numbering of TM regions begins with the most N-terminally disposed TM region (TM1) and concludes with the most C-terminally disposed TM region (TM7).

Accordingly, in many embodiments, the amino acid coordinates of the TM5, IC-3, and TM6 regions of a GPCR are identified by a suitable method such as TMHMM.

In general, once the TM5-IC3-TM6 segment is identified for a parental GPCR, a suitable region of amino acids is chosen for substitution with the amino acid sequence of a GPCR upregulating cassette. In many embodiments, the substituted region is identified using conserved or semi-conserved amino acids in the TM5 and TM6 transmembrane regions. In certain embodiments, the N-terminus of a substituted GURC amino acid sequence (e.g. SEQ ID NO:1 or variant thereof) is juxtaposed to a first amino acid identical to or 1, 2, 3, 4, 5, 6, 7, 8, 9 or 10 residues N-terminal or C-terminal to the amino acid nine residues C terminal to a proline in TM5 of the parental GPCR, and the C-terminus of the substituted GURC amino acid sequence is juxtaposed to a second amino acid identical to or 1, 2, 3, 4, 5, 6, 7, 8, 9 or 10 residues N-terminal or C-terminal to the amino acid twelve residues N-terminal to a proline in TM6 of the parental GPCR. In one embodiment, the N-terminus of the substituted GURC amino acid sequence is juxtaposed to a first amino acid nine residues C-terminal to a proline in TM5 of the parental GPCR, and the C-terminus of the substituted GURC amino acid sequence is juxtaposed to a second amino acid twelve residues N-terminal to a proline in TM6 of the parental GPCR.

For GPCRs of interest that contain no conserved proline residues in TM5 and TM6, positions for inserting a GURC will be determined based on two considerations: a) alignment of the sequence of a GPCR of interest with receptor members of the same subfamily (which contained conserved proline residues in TM5 or TM6; b) by identifying the juxtaposition to TM5/TM6 by hydrophobicity analysis if tested receptors do not belong to any members of GPCRs with conserved proline residues in TM5/TM6

In certain embodiments the two amino acids of the parental GPCR N-terminally flanking the substituted GURC are mutated, e.g. to Gly-Thr (GT). In certain embodiments the two amino acids of the parental GPCR C-terminally flanking the substituted GURC are mutated, e.g. to Glu-Phe (EF).

Polypeptide sequences of 28 exemplary GPCRs that have been modified so as to become ligand upregulatable are shown in FIG. 4 and provided by SEQ ID NOS:10-37.

By way of illustration and not limitation, it can be appreciated that in the substituted niacin receptor of SEQ ID NO:13, the N-terminal arginine of GURC1 amino acid sequence is juxtaposed to an amino acid nine residues C-terminal to a proline in TM5 of the niacin receptor, and the C-terminal leucine of GURC1 amino acid sequence is juxtaposed to an amino acid 12 residues N-terminal to a proline in TM6 of the niacin receptor. The two amino acids of the niacin receptor N-terminally flanking the substituted GURC have been mutated to GT. The two amino acids of the niacin receptor C-terminally flanking the substituted GURC have been mutated to EF.

By way of further illustration and not limitation, it can be appreciated that in the substituted glucagon-like peptide 1 receptor of SEQ ID NO:32, the N-terminal arginine of GURC1 amino acid sequence is juxtaposed to an amino acid 19 residues C-terminal to a proline in TM5 of the glucagon-like peptide 1 receptor, and the C-terminal leucine of GURC1 amino acid sequence is juxtaposed to an amino acid 12 residues N-terminal to a proline in TM6 of the glucagon-like peptide 1 receptor. The two amino acids of the glucagon-like peptide 1 receptor N-terminally flanking the substituted GURC have been mutated to GT. The two amino acids of the glucagon-like peptide 1 receptor C-terminally flanking the substituted GURC have been mutated to EF.

In addition to the methods described, above, one of skill in the art can also perform pairwise or multiple sequence alignments with the parental sequences of the GPCRs shown in FIG. 4, and, using this information, decide which amino acids may be substituted. For example, the most similar parental GPCR could be identified by BLAST, and the region for substitution for a GPCR of interest could be determined by performing a pairwise alignment of that GPCR with the most similar parental GPCR and viewing the ligand upregulatable version of that GPCR to determine which region to substitute.

In certain embodiments the subject invention relates to isolated ligand upregulatable GPCR made by a method of the invention.

GPCR Upregulating Cassettes

A GPCR upregulating cassette is a sequence of amino acids that can be inserted or substituted into a parental GPCR to change the parental GPCR into a ligand upregulatable GPCR

In general, a GPCR upregulating cassette is approximately 30-70 or more amino acids (e.g. about 30, about 35, about 40, about 45, about 50, about 55, about 60, about 65, or about 70 or more) in length, and, in some embodiments, comprises the sequence of SEQ ID NO:1.

When present, SEQ ID NO: 1 is usually positioned within the GPCR upregulating cassette at a position proximal to the C-terminal end of the GPCR upregulating cassette, usually about 1 amino acid, 2 amino acids, 3 amino acids, 4 amino acids, 5 amino acids, or 6, 7, or about 8 amino acids N-terminal to the C-terminus of the GPCR upregulating cassette.

Exemplary GPCR upregulating cassettes have the sequence of SEQ ID NO:2 or a biologically active variant thereof. Variants of SEQ ID NO:2 may contain any combination of insertions, deletions, additions, and amino substitutions, particularly conservative amino acid substitutions as compared to the sequence of SEQ ID NO:2. In the context of a GPCR upregulating cassette, biologically active means having an activity that causes a GPCR to become a ligand upregulatable GPCR.

A GPCR upregulating cassette may contain at least about 10 amino acids, at least about 15 amino acids, at least about 20 amino acids, at least about 25 amino acids, at least about 30 amino acids, at least about 30 amino acids, at least about 30 amino acids, at least about 35 amino acids, at least about 40 amino acids, at least about 45 amino acids or at least about 50 amino acids of SEQ ID NO:2, and in some embodiments comprises the sequence of SEQ ID NO:1.

In certain embodiments, a GPCR upregulating cassette comprises a fragment that contains at least about 70% identity, at least about 75% identity, at least about 80% identity, at least about 85% identity, at least about 90% identity, at least about 95% identity to a fragment of SEQ ID NO:2, where a fragment may be about 10, about 15, about 20, about 25, about 30, about 35, about 40, about 45 or about 50 contiguous amino acids.

An exemplary embodiment of a GPCR upregulating cassette has the sequence of SEQ ID NO:2. SEQ ID NO:2 corresponds to amino acids 221-275 from the native human β2 adrenoreceptor, that has been substituted at four positions proximal to its C-terminal end. The substitutions result in the sequence of SEQ ID NO:1, where the Ser, Arg, the first Lys and the second Ala, are substituted into the sequence of the native human β2 adrenoreceptor.

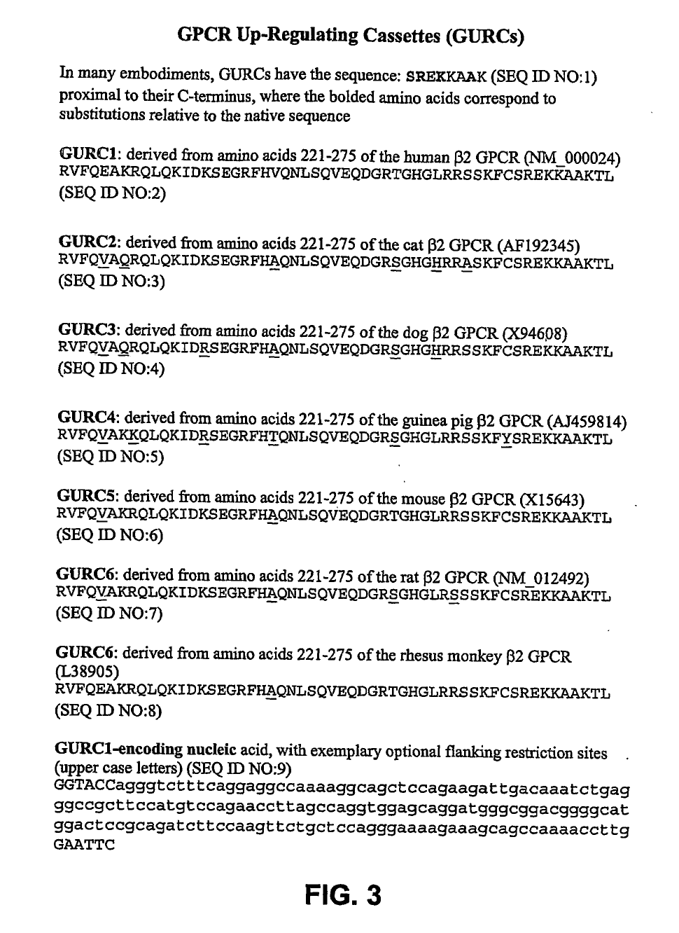

Suitable GPCR upregulating cassettes, are shown in FIG. 3. These GPCR upregulating cassettes are derived from β2 adrenergic GPCRs from various non-human species e.g. those β2 GPCRs identified by GenBank accession numbers AF192345 (cat), X94608 (dog), AJ459814 (guinea pig), X15643 (mouse), rat (NM_{—}012492) and monkey (L38905). In many embodiments, the GURCs of the subject invention comprise the sequence of SEQ ID NO:1. Sequence alignments between these adrenoreceptors would indicate which amino acids could be substituted into the GURCs without them losing biological activity, and, as such, a large number of GURCs could be generated using the subject methods.

Ligand Upregulatable GPCR-Encoding Nucleic Acids

As discussed above, a ligand upregulatable GPCR can be made by substituting a GPCR upregulating cassette amino acid sequence into a parental GPCR at a position within the TM5-IC3-TM6 segment of the parental GPCR. In certain embodiments, standard recombinant DNA technology (Ausubel, et al, Short Protocols in Molecular Biology, 3rd ed., Wiley & Sons, 1995; Sambrook, et al., Molecular Cloning: A Laboratory Manual, Second Edition, (1989) Cold Spring Harbor, N.Y.) methods are used to substitute, delete, and/or add appropriate nucleotides in the nucleic acid sequence encoding a parental GPCR in order to create a ligand upregulatable GPCR-encoding nucleic acid from a parental GPCR-encoding nucleic acid. For example, site directed mutagenesis and subcloning may be used to introduce/delete/substitute nucleic acid residues in the polynucleotide encoding a parental GPCR such that the mutagenized polynucleotide encodes a ligand upregulatable GPCR. In other methods, PCR is used.

Since the genetic code and recombinant techniques for manipulating nucleic acid are known, and the amino acid sequence of GPCRs and GPCR upregulating cassettes are known and described as above, the design and production of nucleic acids encoding a ligand upregulatable GPCR is well within the skill of an artisan.

Said design and production of nucleic acid encoding a ligand upregulatable GPCR encompasses chemical synthesis entirely from oligonucleotides. Synthesis of nucleic acid encoding a ligand upregulatable GPCR by assembly of oligonucleotides is well within the purview of persons of ordinary skill in the art [Cello et al., Science (2002) 297:1016-8; the disclosure of which is hereby incorporated by reference in its entirely].

Exemplary ligand upregulatable GPCR-encoding nucleic acids are provided as SEQ ID NOS: 38-64, which encode the ligand upregulatable GPCRs described by SEQ ID NOS:10-37, respectively.

The invention further provides vectors (also referred to as “constructs”) comprising a subject nucleic acid encoding a ligand upregulatable GPCR. In many embodiments of the invention, nucleic acid sequences encoding a ligand upregulatable GPCR will be expressed in a host after the sequences have been operably linked to an expression control sequence, including, e.g. a promoter. The subject nucleic acids are also typically placed in an expression vector that can replicate in a host cell either as an episome or as an integral part of the host chromosomal DNA. Commonly, expression vectors will contain selection markers, e.g., tetracycline or neomycin, to permit detection of those cells transformed with the desired DNA sequences (see, e.g., U.S. Pat. No. 4,704,362, which is incorporated herein by reference). Vectors, including single and dual expression cassette vectors are well known in the art (Ausubel, et al, Short Protocols in Molecular Biology, 3rd ed., Wiley & Sons, 1995; Sambrook, et al., Molecular Cloning: A Laboratory Manual, Second Edition, (1989) Cold Spring Harbor, N.Y.). Suitable vectors include viral vectors, plasmids, cosmids, artificial chromosomes. (human artificial chromosomes, bacterial artificial chromosomes, yeast artificial chromosomes, etc.), mini-chromosomes, and the like. Retroviral, adenoviral and adeno-associated viral vectors may be used.

A variety of expression vectors are available to those in the art for purposes of utilization for both native and altered native GPCRs. One suitable vector is pCMV, which is used in certain embodiments. This vector was deposited with the American Type Culture Collection (ATCC) on Oct. 13, 1998 (10801 University Blvd., Manassas, Va. 20110-2209 USA) under the provisions of the Budapest Treaty for the International Recognition of the Deposit of Microorganisms for the Purpose of Patent Procedure. The DNA was tested by the ATCC and determined to be viable. The ATCC has assigned the following deposit number to pCMV: ATCC #203351.

The subject nucleic acids usually comprise a single open reading frame encoding the subject ligand upregulatable GPCR, however, in certain embodiments, since the host cell for expression of the ligand upregulatable GPCR may be a eukaryotic cell, e.g., a mammalian cell, such as a human cell, the open reading frame may be interrupted by introns. Subject nucleic acids are typically part of a transcriptional unit which may contain, in addition to the subject nucleic acid, 3′ and 5′ untranslated regions (UTRs) which may direct RNA stability, translational efficiency, etc. The subject nucleic acid may also be part of an expression cassette which contains, in addition to the subject nucleic acid a promoter, which directs the transcription and expression of a ligand upregulatable GPCR-encoding RNA, and a transcriptional terminator.

Eukaryotic promoters can be any promoter that is functional in a eukaryotic host cell, including viral promoters and promoters derived from eukaryotic genes. Exemplary eukaryotic promoters include, but are not limited to, the following: the promoter of the mouse metallothionein I gene sequence (Hamer et al., J. Mol. Appl. Gen. 1:273-288, 1982); the TK promoter of Herpes virus (McKnight, Cell 31:355-365, 1982); the SV40 early promoter (Benoist et al., Nature (London) 290:304-310, 1981); the yeast gall gene sequence promoter (Johnston et al., Proc. Natl. Acad. Sci. (USA) 79:6971-6975, 1982); Silver et al., Proc. Natl. Acad. Sci. (USA) 81:5951-59SS, 1984), the CMV promoter, the EF-1 promoter, Ecdysone-responsive promoter(s), tetracycline-responsive promoter, and the like. Viral promoters may be of particular interest as they are generally particularly strong promoters. In certain embodiments, a promoter is used that is a promoter of the target pathogen. Promoters for use in the present invention are selected such that they are functional in the cell type (and/or animal) into which they are being introduced. In certain embodiments, said promoter is the CMV promoter.

As mentioned above, ligand upregulatable GPCRs may be a made from a parental GPCR that is operably linked to a suitable reporter such as β-galactosidase, luciferase (e.g. Renilla or photonis), β-glucuronidase, chloramphenicol acetyl transferase, and secreted embryonic alkaline phosphatase; proteins for which immunoassays are readily available such as hormones and cytokines; proteins which confer a selective growth advantage on cells such as adenosine deaminase, amino-glycoside phosphotransferase (the product of the neo gene), dihydrofolate reductase, hygromycin-B-phosphotransferase, thymidine kinase (when used with HAT medium), xanthine-guanine phosphoribosyltransferase (XGPRT), and proteins which provide a biosynthetic capability missing from an auxotroph; proteins which confer a growth disadvantage on cells, for example enzymes that convert non-toxic substrates to toxic products such as thymidine kinase (when used with medium containing bromodeoxyuridine) and orotidine-5′-phosphate decarboxylase (when used with 5-fluoroorotic acid). The GPCR can be linked directly to a reporter, or there can be spacer residues between the two (preferably, no more than about 12, although this number can be readily ascertained by one of ordinary skill in the art). Methods for making fusions between a reporter and a GPCR, for example at the C- or N-terminus of the GPCR, are well within the skill of one of skill in the art (e.g. McLean et al, Mol. Pharma. Mol Pharmacol. 1999 56:1182-91; Ramsay et al., Br. J. Pharmacology, 2001, 315-323) and will not be described any further. It is appreciated that a ligand upregulatable GPCR may first be made from a parental GPCR and then operably linked to a suitable reporter as described above. In certain embodiments said reporter is fused to the C-terminus of the ligand upregulatable GPCR. In certain embodiments said reporter is wild type or modified Renilla luciferase.

The subject nucleic acid segments may also contain restriction sites, multiple cloning sites, primer binding sites, ligatable ends, recombination sites etc., usually in order to facilitate the construction of a nucleic acid encoding a modified ligand upregulatable GPCR.

Identification of a Ligand Upregulatable GPCR

In certain embodiments, as a final step in the process of making a ligand upregulatable GPCR, the substituted GPCR (e.g., a parental GPCR that has been altered by substitution within its TM5-IC3-TM6 segment of a GPCR upregulating cassette amino acid sequence) is tested to determine whether its detectable level in or on the host cell surface is increased by ligand. In general, this testing step involves producing the GPCR in a cell, comparing the amount of the substituted GPCR in the presence of a ligand for the GPCR to the amount of the substituted GPCR in the absence of the ligand, and determining whether the substituted GPCR is a ligand upregulatable GPCR.

In most embodiments, a nucleic acid encoding a substituted GPCR is transferred into a cell, and the cell is incubated under conditions sufficient for production of the substituted GPCR. The detectable amounts of the substituted GPCR are then determined, usually separately in the presence and absence of a ligand for the GPCR In most embodiments, the detectable amount of a substituted GPCR in the absence of a ligand is determined, at least qualitatively, ligand is added to the GPCR, and the amount of the substituted GPCR in the presence of the ligand is determined. It is appreciated that said determinations may be made in parallel rather than in series. If a comparison of the amount in the presence of the ligand relative to the amount in the absence of the ligand reveals that there is relatively more substituted GPCR in the presence of the ligand than in the absence of the ligand, the substituted GPCR is a ligand upregulatable GPCR. In most embodiments, a substituted GPCR is a ligand upregulatable GPCR if its amount is at least 10% higher, at least about 30% higher, at least about 50% higher, at least about 70% higher, at least about 100% higher, at least about 150% higher, at least about 200% higher, at least about 300% higher, at least about 400% higher, at least about 500% higher, or, in some embodiments, up to about 1000% or more higher in the presence of a ligand for the GPCR than in the absence of the ligand. As briefly discussed above, a GPCR may be detected by, for example immunological means, and may be operably linked to a epitope, e.g., an HA, V5, c-myc, or FLAG tag to facilitate its detection. In some embodiments, however, a subject GPCR may operably linked to a reporter such that GPCR levels may be determined by measuring the levels of the reporter. In certain embodiments, said reporter is luciferase, e.g. a wild type or modified Renilla luciferase.

Methods of introducing GPCR-encoding nucleic acids into cells are well known in the art. Suitable methods include electroporation, particle gun technology, calcium phosphate precipitation, direct microinjection, and the like. The choice of method is generally dependent on the type of cell being transformed and the circumstances under which the transformation is taking place (i.e. in vitro, ex vivo, or in vivo). A general discussion of these methods can be found in Ausubel, et al, Short Protocols in Molecular Biology, 3rd ed., Wiley & Sons, 1995. In some embodiments lipofectamine and calcium mediated gene transfer technologies are used. Methods for introducing circular nucleic acids are also well known in the art and discussed in Ausubel, above.

Methods of determining reporter activity, e.g. luciferase, GFP, etc., are generally well known in the art (e.g. Ramsay et al., Br. J. Pharmacology, 2001, 133:315-323), and need not be described any further.

Methods for Determining Whether a Test Compound is a Ligand of a GPCR

The invention also provides methods of screening test compounds to identify ligands of a GPCR. In many embodiments, these methods are in vitro methods, involving contacting a cell producing a ligand upregulatable GPCR with a test compound, and determining the amount of the ligand upregulatable GPCR in the presence of the test compound in relation to a suitable control. In many embodiments, a suitable control is the ligand upregulatable GPCR in the absence of the test compound. A ligand usually increases the amount of detectable ligand upregulatable GPCR in comparison to controls. In many embodiments, a ligand increases the amount of detectable ligand upregulatable GPCR by at least about 10%, at least about 20%, at least about 40%, at least about 60%, at least about 80%, at least about 100%, at least about 150%, at least about 200%, at least about 300%, at least about 500%, at least about 10-fold or by at least about 20-fold, or more, as compared to controls.

In an exemplary embodiment, a nucleic acid encoding a ligand upregulatable GPCR is introduced into a suitable cell, and the cell is incubated under conditions that provide for expression of the ligand upregulatable GPCR. The amount of detectable ligand upregulatable GPCR is determined for a cell producing the ligand upregulatable GPCR, or group of such cells, in the presence and in the absence of a test compound. In certain embodiments, the ligand upregulatable GPCR detected on a cell is determined prior to its contact with a test compound, and also determined after the cell has been contacted with the agent, usually at least about 10 minutes, at least about 30 minutes, at least about 1 hr, at least about 2 hr, at least about 4 hr, at least about 8 hr, at least about 12 hr or at least about 24 hr or more after the candidate agent is contacted. In certain embodiments, said determinations are made in parallel rather than in series. Detection of the ligand upregulatable GPCR, as described above, may be done by any suitable method. In most embodiments, detection is done using a quantitative reporter assay using a ligand upregulatable GPCR fused to a reporter, e.g. luciferase, GFP, etc. In certain embodiments said reporter is wild type or modified Renilla luciferase.

The above assays involve expressing a ligand upregulatable GPCR in a suitable host cell, such as a eukaryotic cell e.g. an animal cell (for example an insect, mammal, fish, amphibian, bird or reptile cell), a plant cell (for example a maize or Arabidopsis cell), or a fungal cell (for example a S. cerevisiae cell), and detecting the level of the GPCR in the absence of a candidate agent. Any cell suitable for expression of a ligand upregulatable GPCR-encoding nucleic acid may be used as a host cell. Usually, an animal host cell line is used, examples of which are as follows: monkey kidney cells (COS cells), monkey kidney CVI cells transformed by SV40 (COS-7, ATCC CRL 165 1); human embryonic kidney cells (HEK-293, Graham et al. J. Gen Virol. 36:59 (1977)); HEK-293T cells; baby hamster kidney cells (BHK, ATCC CCL 10); chinese hamster ovary-cells (CHO, Urlaub and Chasin, Proc. Natl. Acad. Sci. (USA) 77:4216, (1980); mouse sertoli cells (TM4, Mather, Biol. Reprod. 23:243-251 (1980)); monkey kidney cells (CVI ATCC CCL 70); african green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (hep G2, HB 8065); mouse mammary tumor (MMT 060562, ATCC CCL 51); TRI cells (Mather et al., Annals N. Y. Acad. Sci 383:44-68 (1982)); NIH/3T3 cells (ATCC CRL-1658); and mouse L cells (ATCC CCL-1). In certain embodiments, melanophores are used. Melanophores are skin cells found in lower vertebrates. Relevant materials and methods will be followed according to the disclosure of U.S. Pat. No. 5,462,856 and U.S. Pat. No. 6,051,386. These patent disclosures are hereby incorporated by reference in their entirety. Additional cell lines will become apparent to those of ordinary skill in the art, and a wide variety of cell lines are available from the American Type Culture Collection, 10801 University Boulevard, Manassas, Va. 20110-2209.

In most embodiments, the assays are performed in a format suitable for high throughput assays, e.g., 96- or 384-well format, and suitable robots, (e.g., pipetting robots), and instrumentation (96- or 384-well format luminometers or fluorescence readers for determining reporter activity) may be used. By way of illustration and not limitation, said determining reporter activity may comprise use of a Wallac 1450 Microbeta counter (Perkin-Elmer). In certain embodiments, said determining reporter activity comprises use of a CCD camera-based illuminator.

A variety of different test compounds may be screened by the above methods. Test compounds encompass numerous chemical classes, though typically they are organic molecules, preferably small organic compounds having a molecular weight of more than 50 and less than about 2,500 daltons. Test compounds comprise functional groups necessary for structural interaction with proteins, particularly hydrogen bonding, and typically include at least an amine, carbonyl, hydroxyl or carboxyl group, preferably at least two of the functional chemical groups. The test compounds often comprise cyclical carbon or heterocyclic structures and/or aromatic or polyaromatic structures substituted with one or more of the above functional groups. Test compounds are also found among biomolecules including peptides, saccharides, fatty acids, steroids, purines, pyrimidines, derivatives, structural analogs or combinations thereof. Further test compounds include variants of the GCPR's native ligand.

Test compounds may be obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

Of interest are test compounds that are polypeptides, e.g., proteinaceous, agents. A specific type of polypeptide test compound of interest is an antibody for the GPCR, or a GPCR-binding fragment thereof. The antibody may be monoclonal or polyclonal, and may be produced according to methods known in the art. Further test compounds include variants of the GCPR's native ligand, e.g. a native ligand that is altered by substitution, deletion or addition of at least one amino acid, or chemically modified. In certain embodiments test compounds include endogenous polypeptides not known to be ligands of the GPCR.

The foregoing characterization of test compounds is intended to be illustrative and not limiting.

Methods for Modulating a GPCR

GPCR ligands identified by methods of the invention encompass modulators of the GPCR. It is well within the purview of persons of ordinary skill in the art to determine whether a ligand of a GPCR is a modulator of the GPCR. Exemplary assays are provided in the Examples.

As such, a test compound identified by a method of the invention using a ligand upregulatable GPCR to be a ligand of a parental GPCR, wherein said ligand is a modulator of the corresponding native GPCR, may be used to modulate the activity of the native GPCR or variant thereof.

The subject methods may be used to identify GPCR ligands that are modulators of the GPCR, e.g. antagonists, agonists, partial agonists or inverse agonists, and, as such, the identified ligand that is a modulator may increase or decrease the activity of a GPCR. GPCR activity may be measured using any suitable activity assay (GTP-binding assay, etc., as described in the Examples).

By way of illustration and not limitation, it can be appreciated that for the [³⁵S]GTPγS assay, an agonist increases binding of [³⁵S]GTPγS to membrane. Both an antagonist and an inverse agonist decrease the level of binding of [³⁵S]GTPγS to membrane mediated by a known agonist. An inverse agonist decreases the agonist-independent activity of a constitutively active native GPCR or of a constitutively activated GPCR, whereas an antagonist does not.

In embodiments where the modulator increases GPCR activity, the activity of the GPCR is increased in the presence of the modulator by at least about 10%, by at least about 20%, by at least about 30%, by at least about 50%, by at least about 80%, by at least about 100%, by at least about 500%, or by at least about 10-fold or more, as compared to suitable controls in the absence of the agent. Suitable controls may be in the presence of absence of the native ligand for the GPCR.

In embodiments where the modulator decreases GPCR activity, the activity of the GPCR is decreases in the presence of the modulator by at least about 10%, by at least about 20% by at least about 30%, by at least about 50%, by at least about 70%, by at least about 80%, by at least about 90%, or by at least about 95% or more, as compared to suitable controls in the absence of the agent. Suitable controls may be in the presence of absence of the native ligand for the GPCR.

In certain embodiments, these methods also involve measuring GPCR activity in the presence or absence of a compound. These activity measurements may involve contacting an isolated cell (e.g., a cultured cell) membrane isolated from a cell, an extract of a cell, or an isolated GPCR with an effective amount of a GPCR modulator to modulate the activity of the GPCR.

In other embodiments, the methods are in vivo methods involving modulating the activity of a GPCR in a mammal, such as a primate (e.g. human, chimpanzee, etc.), rodent (mouse or rat, etc.) or any other animal that is the source of a GPCR, by administering to the mammal an effective amount of a GPCR ligand that is a modulator of the GPCR to modulate the activity of the GPCR in the mammal. In practicing the subject methods, an effective amount of the active agent is administered to the individual, where the term “effective amount” means a dosage sufficient to produce a desired result, where the desired result is the desired modulation, e.g., enhancement, reduction, of at least one GPCR activity.

In practicing the subject in vivo methods, the active compound or compounds are typically administered to the host in a physiologically acceptable delivery vehicle, e.g., as a pharmaceutical preparation. A variety of representative formulations, dosages, routes of administration for candidate agents are described below.

### Formulations, Dosages, and Routes of Administration

The invention provides formulations, including pharmaceutical formulations, that include an agent which modulates GPCR activity in an individual. In general, a formulation comprises an effective amount of an agent that modulates a native GPCR activity in an individual. In many embodiments, the desired result is at least a reduction or increase in a phenotype as compared to a control such that the phenotype is more similar to normal.

Formulations

In the subject methods, the active compound(s) may be administered to the individual using any convenient means capable of resulting in the desired GPCR modulation.

Thus, the compound can be incorporated into a variety of formulations for therapeutic administration. More particularly, the compounds of the present invention can be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments, solutions, suppositories, injections, inhalants and aerosols.

In pharmaceutical dosage forms, the compounds may be administered in the form of their pharmaceutically acceptable salts, or they may also be used alone or in appropriate association, as well as in combination, with other pharmaceutically active compounds. The following methods and excipients are merely exemplary and are in no way limiting.

For oral preparations, the compounds can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

The compounds can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

The compounds can be utilized in aerosol formulation to be administered via inhalation. The compounds of the present invention can be formulated into pressurized acceptable propellants such as dichlorodifluoromethane, propane, nitrogen and the like.

Furthermore, the compounds can be made into suppositories by mixing with a variety of bases such as emulsifying bases or water-soluble bases. The compounds of the present invention can be administered rectally via a suppository. The suppository can include vehicles such as cocoa butter, carbowaxes and polyethylene glycols, which melt at body temperature, yet are solidified at room temperature.

Unit dosage forms for oral or rectal administration such as syrups, elixirs, and suspensions may be provided wherein each dosage unit, for example, teaspoonful, tablespoonful, tablet or suppository, contains a predetermined amount of the composition containing one or more inhibitors. Similarly, unit dosage forms for injection or intravenous administration may comprise the inhibitor(s) in a composition as a solution in sterile water, normal saline or another pharmaceutically acceptable carrier.

The term “unit dosage form,” as used herein, refers to physically discrete units suitable as unitary dosages for human and animal subjects, each unit containing a predetermined quantity of compounds of the present invention calculated in an amount sufficient to produce the desired effect in association with a pharmaceutically acceptable diluent, carrier or vehicle. The specifications for the novel unit dosage forms of the present invention depend on the particular compound employed and the effect to be achieved, and the pharmacodynamics associated with each compound in the host.

Other modes of administration will also find use with the subject invention. For instance, a compound of the invention can be formulated in suppositories and, in some cases, aerosol and intranasal compositions. For suppositories, the vehicle composition will include traditional binders and carriers such as, polyalkylene glycols, or triglycerides. Such suppositories may be formed from mixtures containing the active ingredient in the range of about 0.5% to about 10% (w/w), preferably about 1% to about 2%.

Intranasal formulations will usually include vehicles that neither cause irritation to the nasal mucosa nor significantly disturb ciliary function. Diluents such as water, aqueous saline or other known substances can be employed with the subject invention. The nasal formulations may also contain preservatives such as, but not limited to, chlorobutanol and benzalkonium chloride. A surfactant may be present to enhance absorption of the subject proteins by the nasal mucosa.

An compound of the invention can be administered as injectables. Typically, injectable compositions are prepared as liquid solutions or suspensions; solid forms suitable for solution in, or suspension in, liquid vehicles prior to injection may also be prepared. The preparation may also be emulsified or the active ingredient encapsulated in liposome vehicles.

Suitable excipient vehicles are, for example, water, saline, dextrose, glycerol, ethanol, or the like, and combinations thereof In addition, if desired, the vehicle may contain minor amounts of auxiliary substances such as wetting or emulsifying agents or pH buffering agents. Actual methods of preparing such dosage forms are known, or will be apparent, to those skilled in the art. See, e.g., Remington's Pharmaceutical Sciences, Mack Publishing Company, Easton, Pennsylvania, 17th edition, 1985; Remington: The Science and Practice of Pharmacy, A. R. Gennaro, (2000) Lippincott, Williams & Wilkins. The composition or formulation to be administered will, in any event, contain a quantity of the agent adequate to achieve the desired state in the subject being treated.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

Dosages

Although the dosage used will vary depending on the clinical goals to be achieved, a suitable dosage range is one which provides up to about 1 μg to about 1,000 μg or about 10,000 μg of an compound that reduces a symptom of a GPCR-related disorder in an individual.

Those of skill will readily appreciate that dose levels can vary as a function of the specific compound, the severity of the symptoms and the susceptibility of the subject to side effects. Preferred dosages for a given compound are readily determinable by those of skill in the art by a variety of means.

Routes of administration

Conventional and pharmaceutically acceptable routes of administration include intranasal, intramuscular, intratracheal, intratumoral, subcutaneous, intradermal, topical application, intravenous, rectal, nasal, oral and other parenteral routes of administration. Routes of administration may be combined, if desired, or adjusted depending upon the compound and/or the desired effect. The composition can be administered in a single dose or in multiple doses.

The compound can be administered to a host using any available conventional methods and routes suitable for delivery of conventional drugs, including systemic or localized routes. In general, routes of administration contemplated by the invention include, but are not necessarily limited to, enteral, parenteral, or inhalational routes.

Parenteral routes of administration other than inhalation administration include, but are not necessarily limited to, topical, transdermal, subcutaneous, intramuscular, intraorbital, intracapsular, intraspinal, intrasternal, and intravenous routes, i.e., any route of administration other than through the alimentary canal. Parenteral administration can be carried to effect systemic or local delivery of the compound. Where systemic delivery is desired, administration typically involves invasive or systemically absorbed topical or mucosal administration of pharmaceutical preparations.

The agent can also be delivered to the individual by enteral administration. Enteral routes of administration include, but are not necessarily limited to, oral and rectal (e.g., using a suppository) delivery.

Methods of administration of the compound through the skin or mucosa include, but are not necessarily limited to, topical application of a suitable pharmaceutical preparation, transdermal transmission, injection and epidermal administration. For transdermal transmission, absorption promoters or iontophoresis are suitable methods. Iontophoretic transmission may be accomplished using commercially available “patches” which deliver their product continuously via electric pulses through unbroken skin for periods of several days or more.

By treatment is meant at least an amelioration of the symptoms associated with the pathological condition afflicting the host, where amelioration is used in a broad sense to refer to at least a reduction in the magnitude of a parameter, e.g. symptom, associated with the pathological condition being treated. As such, treatment also includes situations where the pathological condition, or at least symptoms associated therewith, are completely inhibited, e.g. prevented from happening, or stopped, e.g. terminated, such that the host no longer suffers from the pathological condition, or at least the symptoms that characterize the pathological condition.

Utility

The subject compositions and methods of modulating the activity of a GPCR find use in a variety of therapeutic protocols. In general, these protocols involve administering to an individual suffering from a GPCR-related disorder an effective amount of one or more active compounds that modulate a GPCR to modulate the GPCR in the host and treat the individual for the disorder.

In some embodiments, where a reduction in activity of a certain GPCR is desired, one or more compounds that decrease the activity of the GPCR may be administered, whereas when an increase in activity of a certain GPCR is desired, one or more compounds that increase the activity of the GPCR activity may be administered.

A variety of individuals are treatable according to the subject methods. Generally such individuals are mammals or mammalian, where these terms are used broadly to describe organisms which are within the class mammalia, including the orders carnivore (e.g., dogs and cats), rodentia (e.g., mice, guinea pigs, and rats), and primates (e.g., humans, chimpanzees, and monkeys). In many embodiments, the individuals will be humans.

Subject treatment methods are typically performed on individuals with such disorders or on individuals with a desire to avoid contracting such disorders. The invention also includes preventing or reducing the risk of a GPCR-related condition by administering a pharmaceutical composition comprising a modulator selective for the GPCR.

Kits

Also provided by the subject invention are kits for practicing the subject methods, as described above. The subject kits at least include one or more of: a nucleic acid encoding a G-protein coupled receptor upregulating cassette or a ligand upregulatable GPCR or cells producing a ligand upregulatable GPCR. The nucleic acids of the kit may also have restrictions sites, multiple cloning sites, primer sites, etc to facilitate their ligation other plasmids. Other optional components of the kit include: restriction enzymes, control nucleic acid encoding a G-protein coupled receptor upregulating cassette or a ligand upregulatable GPCR, and buffers, cells etc for performing the subject assays. The various components of the kit may be present in separate containers or certain compatible components may be precombined into a single container, as desired.

In addition to above-mentioned components, the subject kits typically further include instructions for using the components of the kit to practice the subject methods. The instructions for practicing the subject methods are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging) etc. In other embodiments, the instructions are present as an electronic storage data file present on a suitable computer readable storage medium, e.g. CD-ROM, diskette, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the subject invention.

### Materials and Methods

Abbreviations: AR: Adrenergic receptor. Rlu: Renilla luciferase. GPCR: G protein-coupled receptor. NMS: N-methyl scopolamine. Rm3: rat muscarinic acetylcholine receptor 3.

Materials: All materials for cell culture, general molecular biology experiments were supplied by Invitrogen (Carlsbad, Calif.). [³H]N-methyl scopolamine chloride (NMS) (80 Ci/mmol) was from NEN Life Science Products (Boston, Mass.). All chemicals, LOPAC known compound library were obtained from Sigma (St. Louis, Mo.). Oligonucleotides were obtained from Genset (San Diego, Calif.). MG132 (carbobenzoxy-L-leucyl-L-leucyl-L-leucinal) was purchased from Calbiochem (La Jolla, Calif.). Native coelenterazine was from Biotum (Hayward, Calif.). Renilla luciferase-containing vector plasmid pRLCMV was obtained from Promega (Madison, Wis.). Luciferase reporter gene assay kit (LucLite™) was from Packard (Meriden, Conn.).

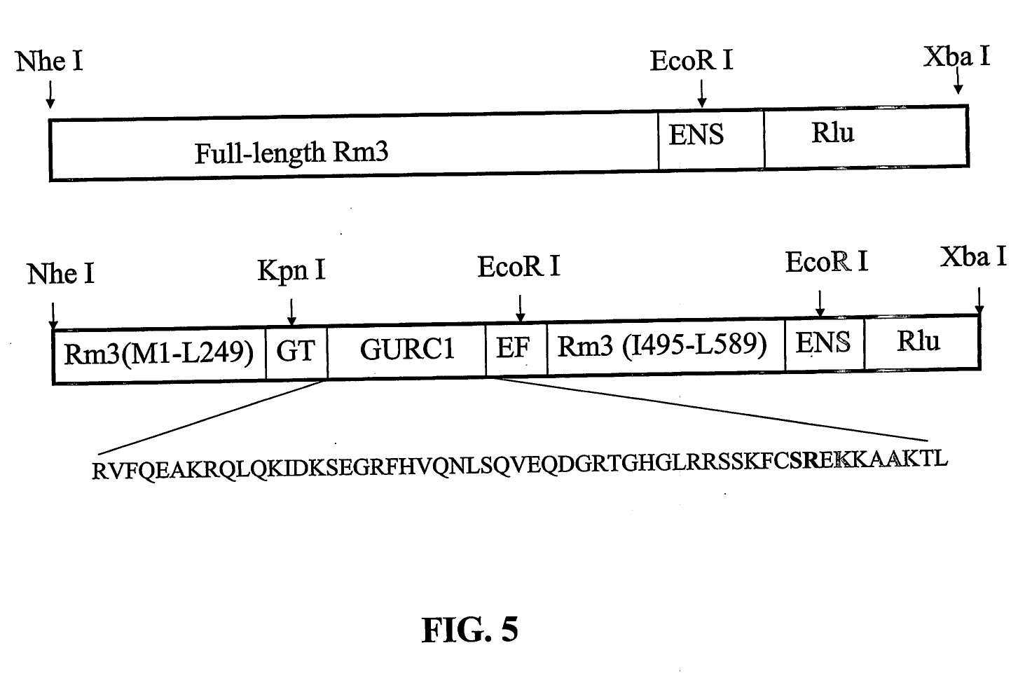

Construction of fusion Rm3 receptor genes: Both rat wild-type Rm3-Rlu and Rm3(GURC)-Rlu were generated as described in FIG. 5. Briefly, Rm3-Rlu was constructed in two steps. First, a full-length cDNA encoding Renilla luciferase (Rlu; 312 amino acids), generated by PCR amplification of a Renilla luciferase-containing vector plasmid pRLCMV, was digested with EcoR I and Xba I, and 1 kb EcoR I-Xba I fragment was subcloned into pCDNA3.1(+) vector, resulting in Rlu-pCDNA3.1. In the second step, a gene coding a full-length rat muscarinic acetylcholine receptor subtype 3 (Rm3) was generated by PCR amplification of Rm3-pCD plasmid (kindly provided by Dr. J. Wess of the National Institute of Health). The PCR product, after digested with Nhe I and EcoR I, was inserted into Rlu-pCDNA3.1 by Nhe I and EcoR I, resulting in final construct Rm3-Rlu. Introduction of EcoR I restriction site between C-terminus of Rm3 and N-terminus of Rlu resulted in insertion of three additional amino acids link Glu-Asn-Ser (FIG. 5).

An Rm3(GURC1)-Rlu, wherein amino acids 250-494 of Rm3 were replaced with GURC1 (see FIGS. 3 and 5), was constructed as follows. In the first step, a PCR fragment encoding GURC1 was inserted into Rlu-pCDNA3.1 with Kpn I and EcoR I sites, resulting in GURC1-Rlu. Secondly, a PCR product encoding amino acids 1-249 of Rm3 (from N-terminal Met to the junction of TMV and IC-3) was subcloned into the upstream of GURC1-Rlu by Nhe I and Kpn I sites. Finally, a PCR product encoding amino acids 495-589 of Rm3 was inserted by non-directional cloning with EcoR I site. Constructs in each step were then verified by DNA sequence analysis. In this construct mutations were introduced at two positions. Introducing Kpn I site resulted in mutation of two amino acids N-terminally flanking the introduced GURC1 (YW to GT) and adding an EcoR I site C-terminally flanking the introduced GURC1 changed SA in Rm3 to EF.

Cell culture and transfection: HEK 293 or COS-7 cells were grown in Dulbecco's modified Eagle's medium (DMEM) supplemented with 10% fetal calf serum, 2 mM L-glutamine, 100 μg/ml penicillin and 100 units/ml streptomycin at 37° C. in a humidified 5% CO₂ incubator. For transfection, 1×10⁶ cells were seeded into 100-mm dishes and 24 h later cells were transfected with 4 μg of plasmid DNA/dish using LipofectAmine™ reagent according to the manufacturer's instructions. To generate cell lines stably expressing receptors, cells were seeded/diluted 2 days after transfection and maintained in DMEM supplemented with 500 μg/ml Geneticin sulfate. The medium was replaced every three days with DMEM supplemented with 500 μg/ml Geneticin sulfate until cells were completely resistant to Geneticin sulfate. To determine the effect of compounds on the steady state receptor expression, cells were split 20 h after transfection into the polylysine-pretreated 96-well plates (Greiner America Inc.), and treated with test compounds at indicated concentration for 20-24 h before luciferase activity assay.

Luciferase activity assay: The non-treated or drug-treated cells were washed once with phosphate-buffed saline solution (PBS). Cells were incubated with 50 μl of lysis buffer (0.25% NP-40 in assay buffer: 100 mM sodium phosphate, pH 7.4, 500 mM NaCl) at room temperature for 30 min. The Renilla luciferase activity was immediately measured by Wallac 1450 Microbeta counter (Perkin-Elmer) after adding 100 μl of 2 μM coelenterazine in assay buffer.

Ligand binding assays: COS-7 cells were transiently transfected with various constructs. After 2 days transfection, cells were washed once with ice-cold PBS. Cells were then detached with PBS/0.5 mM EDTA and resuspended in ice-cold binding buffer (25 mM sodium phosphate, pH 7.4, 2 mM EDTA and 10 mM MgCl₂). Cells were homogenized with 3×10 s bursts of a polytron. The crude cell homogenate was used for following [³H]NMS binding assay. To study the effect of ligands on the expression of Rlu-tagged fusion receptors, cells were treated overnight (20 h) with 1 μM of atropine after 24 h transfection. Non-treated or drug-treated cells were extensively washed with ice-cold PBS to remove the bound drug. Binding assays were carried out with cell homogenates using [³H]NMS as a radioligand. Samples (150 μl) were incubated for 2 h at room temperature in a 96-well plate in binding buffer. To measure maximal saturation binding sites, 2 nM [³H]NMS were used. In competition binding experiments the membrane homogenates were pre-incubated with various concentrations of ligands at room temperature for 30 min prior to addition of 0.5 nM [³H]NMS. Non-specific binding was determined in the presence of 10 μM atropine. Reactions were terminated after 90-min incubation at room temperature. Samples were harvested by rapid filtration through Whatman GF/C 96-well filters followed by three washes with ice-cold wash buffer (50 mM Tris-HCl, 1 mM EDTA, pH 7.4). Once soaked in scintillation fluid, filters were counted in a Packard scintillation counter (Hewlett Packard, Palo Alto, Calif.).

Reporter gene assays: NFAT-reporter gene was used to measure Gq-mediated intracellular calcium change in HEK293 cells. HEK293 cells were seeded in 96-well white plate (10,000 cells/well). After 24 h cells were co-transfected with receptor plasmid and reporter gene plasmid (pNFAT-luc) with LipofectAmine™ 2000 reagent. Cells were changed to phenol red-free, serum-free medium and incubated in the absence or presence of drugs for another 12-16 h. Luciferase activity was then measured with luciferase reporter gene assay kit LucLite™ according to manufacturer's instructions.

Screening of LOPAC compound collection: all compounds were dissolved in DMSO at 400 μM and plated in columns 2-11 of 96-well plates. Atropine (400 μM in DMSO) was plated in column 1 and DMSO in column 12. One microliter was transferred to assay plate containing 100 μl of normal cell culture medium by MiniTrak (Packard BioScience Inc.). HEK293 cells stably expressing Rm3(GURC)-Rlu (100 μl containing about 50,000 cells) were added into assay plate by Multidrop (LabSystems) to make final medium volume 200 μl and final DMSO concentration of 0.5%. Cells were incubated for another 20 h and medium was removed by aspiration. All liquid reagents were handed by Multidrop. Luciferase activity was determined as described above.

Miscellaneous methods: protein was determined according to the method of Bradford using bovine serum albumin as the standard. All data were analyzed by nonlinear least squares curves-fitting procedure, using the computer program GraphPad Prism (San Diego, Calif.).

### Example 1

### Rm3(GURC)-Rlu Fusion Protein Displays Lower Protein Expression Level than the Wild-Type Receptor

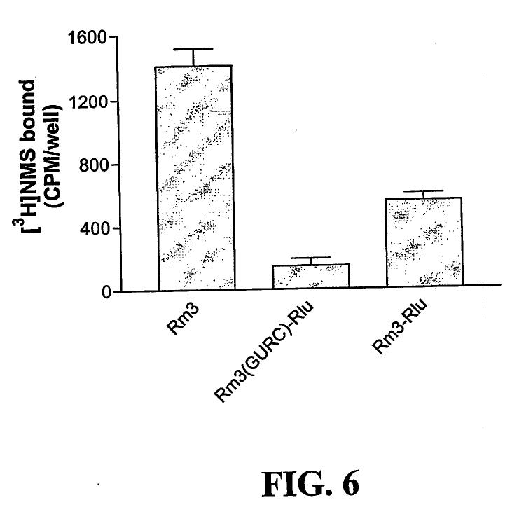

The luciferase gene from the sea pansy Renilla reniforms was fused in-frame to the C-terminus of the wild-type Rm3. Fusion protein displayed a similar binding affinity for the antagonist [³H]NMS to the non-tagged wild-type receptor (data not shown). However, saturation binding studies showed that Rlu-tagged receptor had at least a 2-fold decrease in [³H]NMS binding sites compared to the non-tagged wild-type receptor (FIG. 6). Replacement of amino acids 250-494 with GURC1 as described in Materials and Methods further decreased [³H]NMS binding sites by about 2-fold compared to Rm3-Rlu [FIG. 6. Rm3(GURC)-Rlu]. Overall, Rm3(GURC)-Rlu exhibited at least 4-fold decrease in steady-state protein expression level. These results show that substitution of GURC1 at a position within the TM5-IC3-TM6 segment and addition of Rlu to the C-terminus of receptor generated a mutant receptor with lower steady protein expression level.

### Example 2

### Rm3(GURC)-Rlu Fusion Receptor Retains the Same Binding Affinity to Agonist and Antagonist as the Wild-Type Receptor

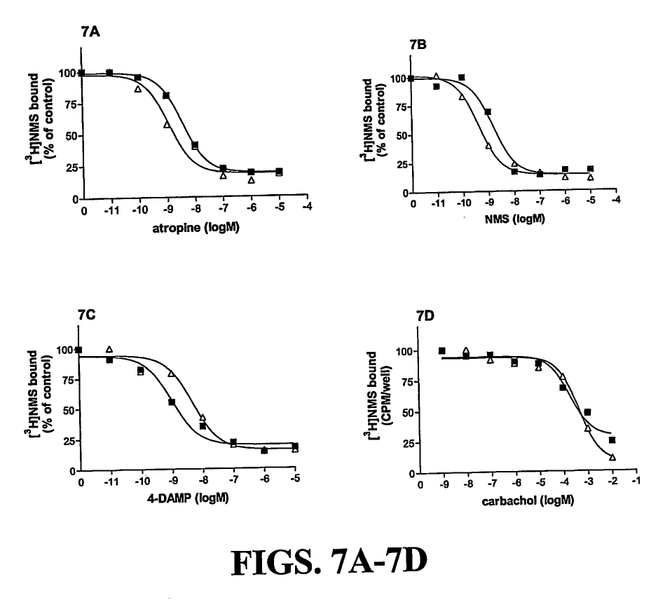

Competition ligand binding experiments were performed with both agonist and antagonists. As shown in FIGS. 7A and 7B, antagonists atropine and NMS showed a slight increase (1-2 fold) in binding affinity to Rm3(GURC)-Rlu than the wild-type receptor (left-shift of competition binding curves). However, antagonist 4-DAMP had a 1-fold decrease in binding affinity to the Rm3(GURC)-Rlu than the wild-type (FIG. 7C). Agonist carbachol showed a similar affinity to wild-type and to the chimera fusion receptor (FIG. 7D). Thus, Rm3(GURC)-Rlu fusion receptor retained ligand (agonist and antagonist) binding properties of the wild-type.

### Example 3

### Ligand Specifically Induces Upregulation of Rm3(GURC)-Rlu

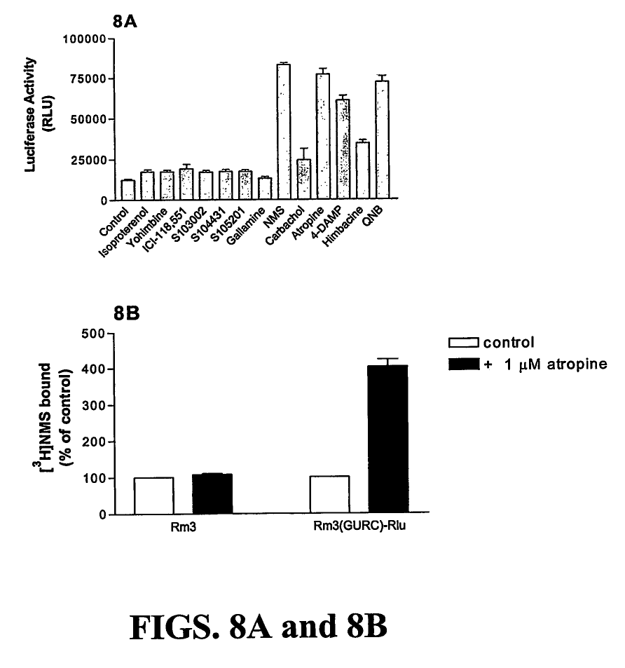

The addition of Rlu to the receptors facilitates the determination of fusion receptor expression by directly measuring luciferase activity. As shown in FIG. 8A, all antagonists (10 μM specific for muscarinic acetylcholine receptors (NMS, atropine, 4-DAMP, himbacine and QNB) led to a 4-5 fold increase in luciferase activity of Rm3(GURC)-Rlu compared to untreated cells. The agonist carbachol (10 μM) caused about 2-fold increase in luciferase activity for Rm3(GURC)-Rlu (FIG. 8A). In contrast, under the same conditions the adrenergic receptors-specific ligands (isoproterenol, yohimbine, ICI-118,551) and other non-ligand compounds (S103002, S104431 and S105201) had no significant effect on the luciferase activity of this fusion receptor. Moreover, other compounds known not to interact with the muscarinic receptors such as ones specific for serotonin receptors (serotonin, metergoline) and histamine receptors (imetit, ranitidine) did not change the luciferase activity (data not shown). These results show that upregulation of the enzyme activity of this fusion receptor was ligand-specific. To exclude the possibility that these ligands directly affected the luciferase enzyme activity, cells were firstly transfected with the wild-type Rlu gene before exposure to test compounds and in a second experiment, the ligands were introduced directly into a luciferase activity assay. In both cases all ligands had no effect on luciferase enzyme activity (data not shown).

### Example 4

### Verification of Ligand-Induced Receptor Upregulation by Radioligand Binding Assay

To verify that the ligand-induced increase in luciferase activity was due to the increase in receptor number, we determined the receptor expression levels of non-treated and ligand-treated cells by measuring [³H]NMS binding sites in ligand binding assay. Treatment of cells expressing Rm3(GURC)-Rlu for 20 h with 1 μM atropine led to about a 4-fold increase in [³H]NMS binding sites (FIG. 8B). Similar treatment of wild-type receptor Rm3-Rlu with atropine had no significant effect on [³H]NMS binding sites. These results are consistent with those obtained in luciferase assays (FIG. 8A), confirming that the increased luciferase activity was due to an actual increase in receptor amount.

### Example 5

### Time- and Dose-Dependence of Ligand-Induced Rm3(GURC)-Rlu Upregulation

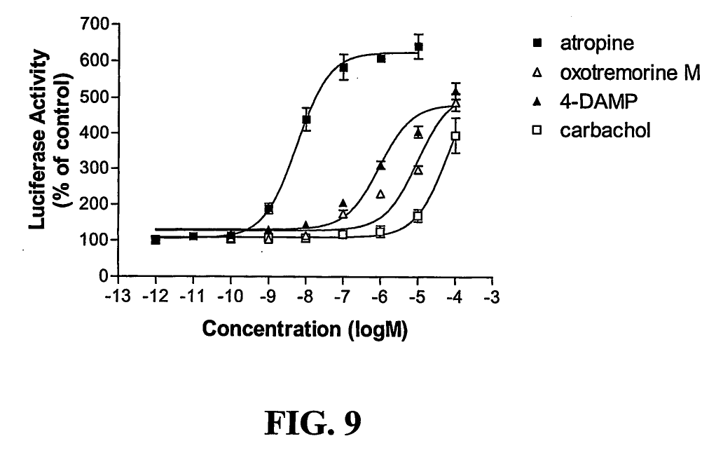

Ligand-induced upregulation of Rm3(GURC)-Rlu were both time-dependent and dose-dependent. Significant upregulation could be detected after at least 6 h-incubation at 37° C. When treated with 10 μM atropine the luciferase activity increased with time course increase from 6 h to 96 h (data not shown). Generally, 20-24 h-incubation time period was chosen for experiments considering compound instability under 37° C. and long-term incubation. As shown in FIG. 9, atropine and 4-DAMP increased luciferase activity of Rm3(GURC)-Rlu in a concentration-dependent manner. All other antagonists tested also showed dose-dependent upregulation for this chimera fusion receptor (data not shown). However, potency (EC₅₀ values) for these ligands was significantly different from one antagonist to another when compared to their Kᵢ values obtained from other assays such as competition binding assays (Pig. 7). EC₅₀ value for atropine (˜6 nM) in luciferase upregulation assay (FIG. 9) was only about 5-fold higher than that of competition binding assay (1.3 nM). Similar small right-shift in EC₅₀ values in this assay was observed for several other antagonists/inverse agonists such as NMS, QNB and Himbacine (data not shown). However, EC₅₀ value for 4-DAMP (˜900 nM) in luciferase upregulation assay was about 1000-fold higher than that of competition binding assay (˜1 nM) (FIG. 7). Agonist carbachol also induced receptor upregulation in dose-dependent manner (FIG. 9), but its EC₅₀ (>100 μM) had at least 10-fold right shift compared to competition binding assay (FIG. 6). Oxotremorine M exhibited an EC₅₀ with 1000-fold higher than their Kᵢ values (FIG. 9). This right-shift of EC₅₀ values in luciferase upregulation assay may be due to ligand instability and agonist-induced receptor down-regulation under assay conditions (37° C. and>20 h treatment) (also see discussion). Indeed, we found that 4-DAMP was less stable than atropine under these conditions, as demonstrated in experiments that detected binding affinity decrease of ligands after treated under the same conditions as in upregulation assay (data not shown).

### Example 6

### Development of a Screening Platform Based on Ligand-Specific Upregulation Of Rm3(GURC)-Rlu

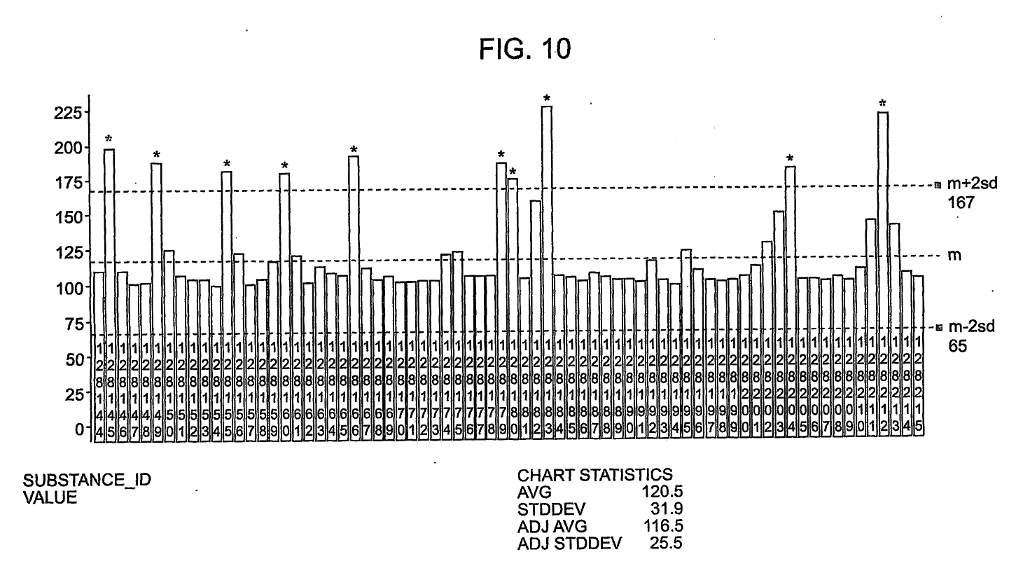

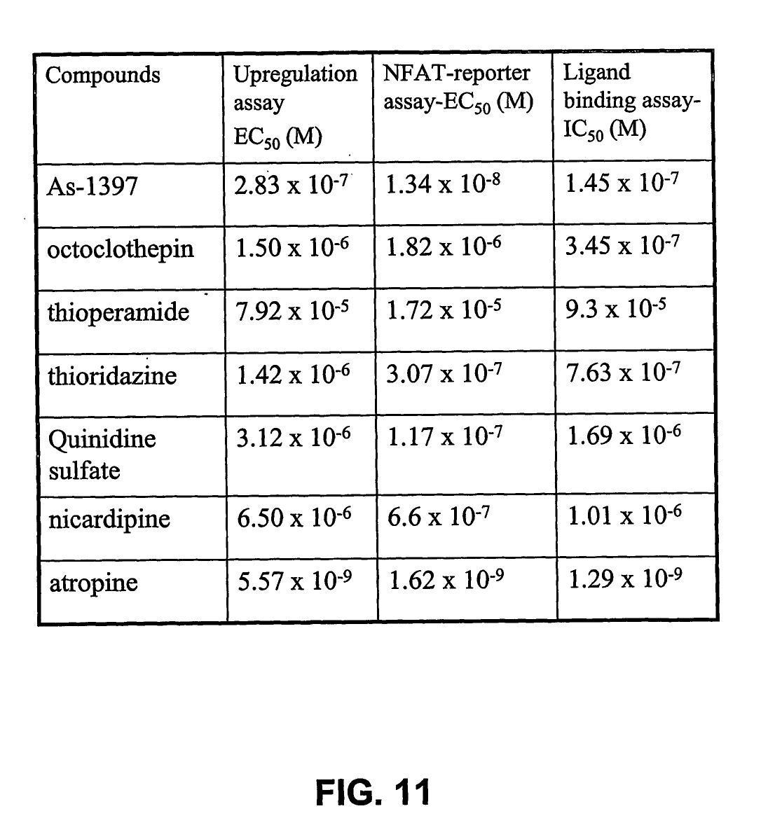

An assay in both 96-well and 384-well plate formats by screening a LOPAC known compound library (about 700 compounds) which included many ligands for muscarinic receptors was established. DMSO up to 1% in concentration did not affect this assay. All compounds were screened at 2 μM concentration and single point with final 0.5% DMSO in 96- and 384 formats. Atropine (2 μM) was used as positive controls and set as 200% response, DMSO alone was negative control (100% response). Both formats generated similar standard deviations (<10%) and Z′ values (0.5-0.7) (data not shown). An example of screening plate output (96-well) is shown in FIG. 10. All compounds that gave greater than 130% response have been selected for reconfirmation assay performed in triplicate. The reconfirmation rate was generally>75%. Known Rm3 ligands could be identified with great accuracy (FIG. 10). Ligand with low affinity (Kᵢ>2 μM) (such as carbachol) and/or instability (such as acetylcholine) could not be identified with the 130% cut-off. Several compounds that displayed very strong activity (>150% response) have not been reported as Rm3 ligands (FIG. 11). These compounds included octoclothepin maleate (D2 dopamine receptor antagonist), thioperamide (H3 histamine receptor antagonist), As-1397 (selective butyrylcholinesterase inhibitor), thioridazine (dopamine receptor antagonist), quinidine sulfate (Na⁺ channel blocker) and nicardipine (Ca²⁺ channel antagonist). These compounds were subjected to competition ligand binding assay and functional NFAT-reporter gene assay with wild-type Rm3 to directly verify their interaction with the receptor. Indeed, all these compounds could inhibit [³H]NMS binding and carbachol-stimulated NFAT gene expression in dose-dependent manner (FIG. 11). EC₅₀ values of these compounds in upregulation assay are generally consistent with those from competition binding assay or NFAT-reporter gene assay. We also screened 40,000 other compounds and several hits were identified and verified in competition [³H]NMS binding assay and phosphatidylinositol hydrolysis assay (data not shown). These results demonstrated that this assay could identity hits with great accuracy and reproducibility in both 96- and 384-format.

### Example 7

### Other Ligand Upregulatable G Protein-Coupled Receptors of the Invention

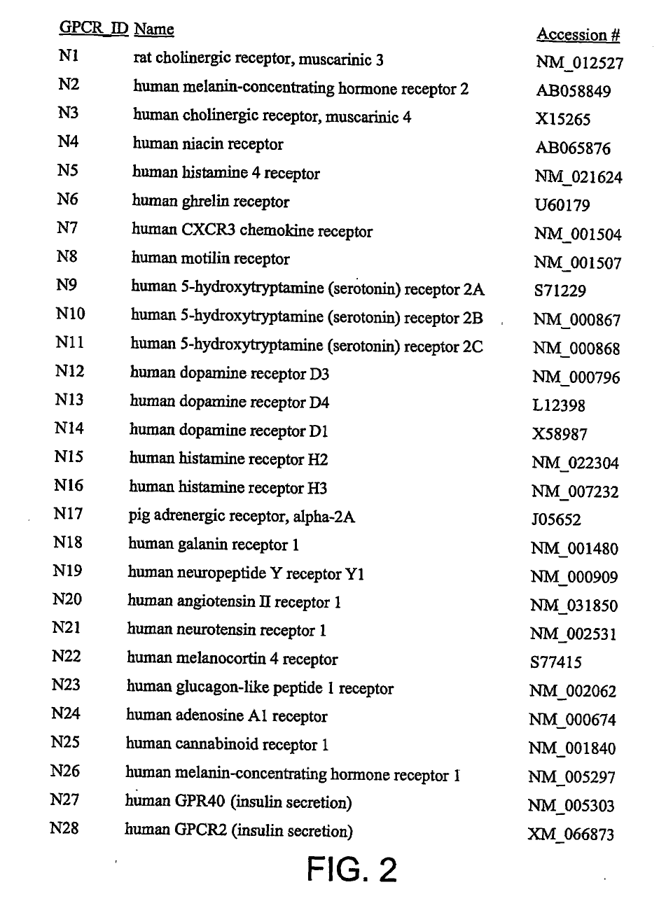

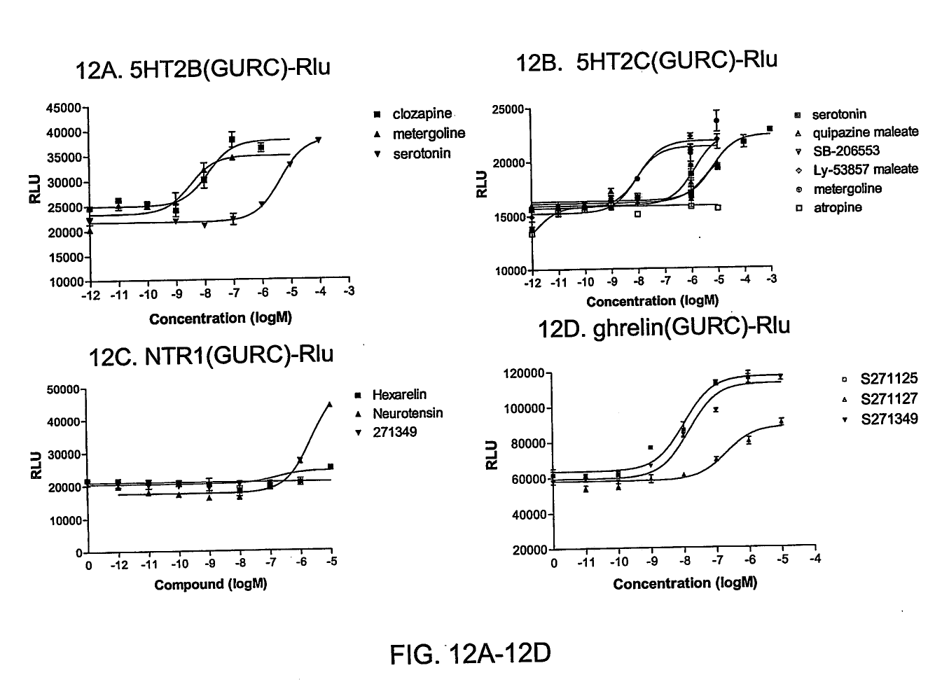

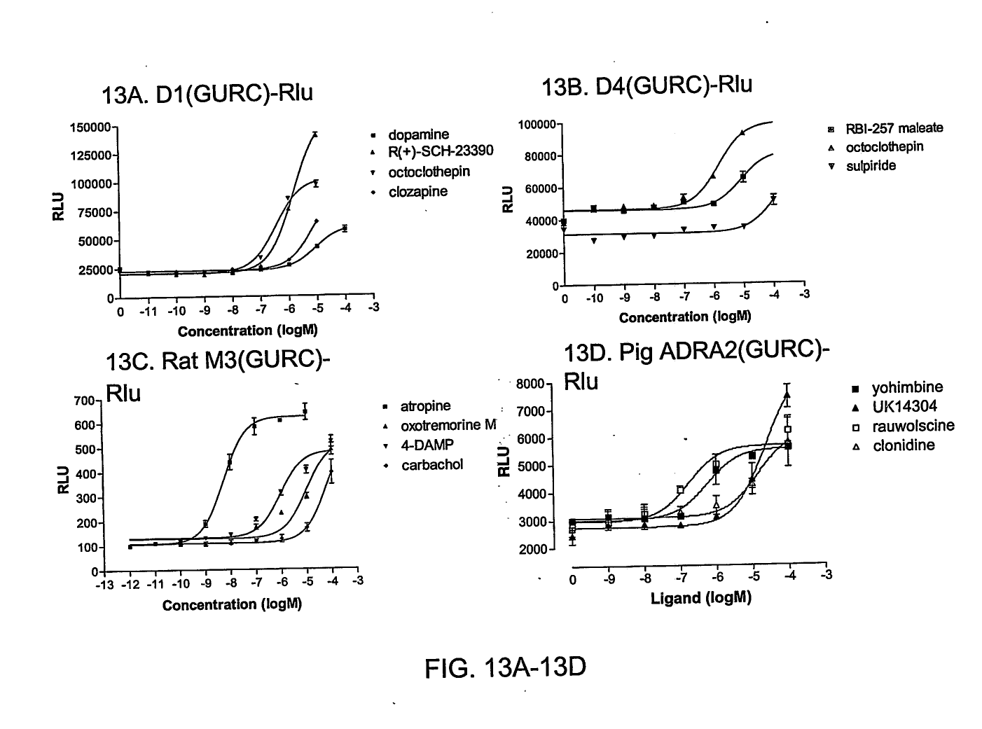

To investigate the general applicability of ligand upregulatable GPCRs constructed by methods of the invention to assay development, the same approach used to design Rm3(GURC)-Rlu was applied to the GPCRs described in FIG. 2, and others. All of the substituted GPCRs were tested and displayed ligand-specific upregulation. Data for eight of the substituted GPCRs are presented in FIGS. 12 and 13 and show that the substituted GPCRs are upregulated by ligand, either agonist or antagonist.

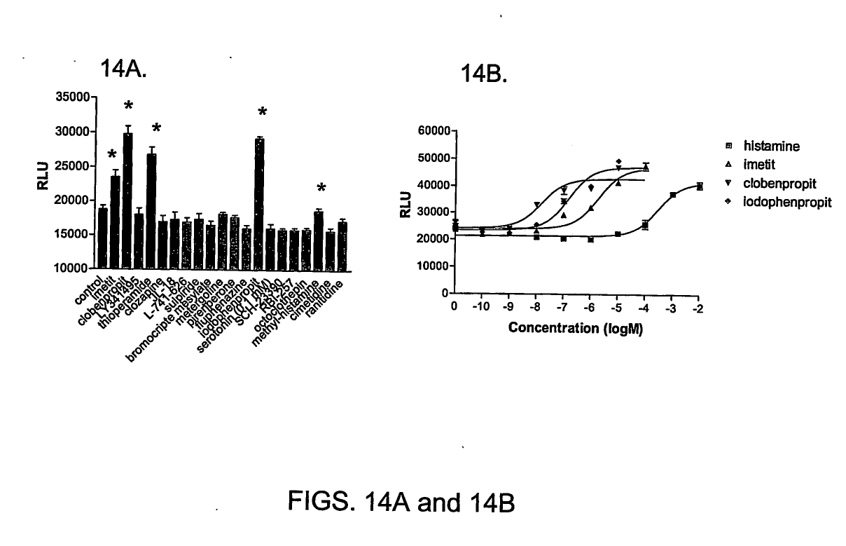

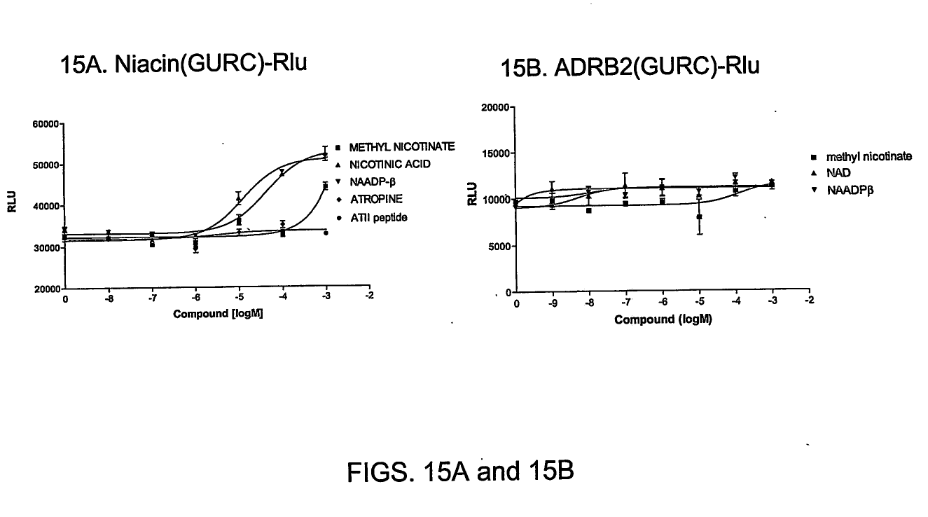

Ligand-specific upregulation of the histamine receptor H3. Using the methods of the invention, the histamine receptor H3 was made ligand upregulatable and tested against a variety of agonists, antagonists, and non-ligands. FIG. 14 displays these results. Imetit and methyl-histamine are agonists. Clobenpropit, thioperamide and iodophenopropin are antagonists. The other compounds are not ligands of histamine receptor H3. The results demonstrate that the histamine receptor H3 manifests ligand-specific upregulatation. Methyl histamine, used in these experiments, is low potency.

Receptor-specific upregulation by niacin receptor ligand. In accordance with methods of the subject invention, the niacin receptor and the β2 adrenergic receptor were also made ligand upregulatable and tested against a panel of agonists, antagonists, and non-ligands. FIG. 15 displays these results. Methyl-nicotinate, nicotinic acid, and NAADP are agonists of the niacin receptor. Atropine and ATII peptide are not ligands of the niacin receptor. None of the indicated compounds is a ligand of the β2-adrenergic receptor. The results demonstrate that the niacin receptor is ligand upregulatable , and that the ligands for the modified niacin receptor fail to upregulate the analogously modified β2 adrenergic receptor.

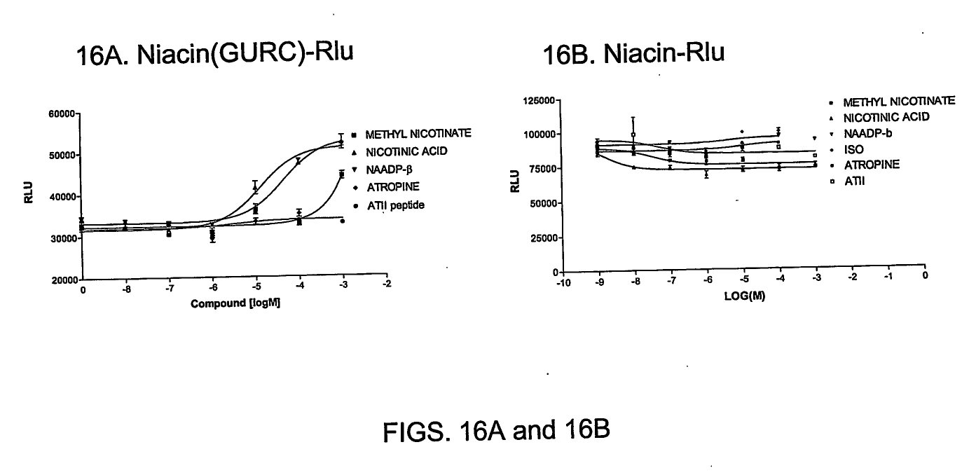

Ligand upregulation of the niacin receptor is dependent on the substituted GURC. In accordance with methods of the subject invention, two modified niacin receptors were made: one with and one without a GURC. As shown in FIG. 16, only the niacin receptor containing a GURC was upregulatable by ligand, showing that it is the GURC that imparts ligand upregulatability to a GPCR.

These results demonstrate the general applicability of this strategy for identifying ligands of a GPCR, wherein said identified ligands encompass modulators of the GPCR, independently of a conventional secondary messenger assay.

### Example 8

### Chemical Synthesis of Nucleic Acid Encoding a Ligand Upregulatable GPCR

Nucleic acid encoding a ligand upregulatable GPCR made by a method of the invention can be constructed by chemical synthesis. Said methods of chemical synthesis are well within the purview of persons of ordinary skill in the art. An exemplary method is that of Cello et al. [Science (2002) 297:1016-8; the disclosure of which is hereby incorporated by reference in its entirety].

Nucleic acid encoding a ligand upregulatable GPCR may be designed on the basis of knowledge of the genetic code and knowledge of the amino acid sequence of the parental GPCR of interest and of the GPCR upregulating cassette (GURC) of interest.

Said nucleic acid can be chemically synthesized using a strategy of assembly of large overlapping nucleic acid fragments which, in turn, are obtained by combining overlapping segments of 400 to 600 base pairs. The segments are synthesized by assembling purified oligonucleotides.

In certain embodiments the parental GPCR is a native GPCR or an altered native GPCR.

In certain embodiments, the parental GPCR is a known GPCR or an altered known GPCR.

In certain embodiments, the parental GPCR is an orphan GPCR or an altered orphan GPCR.

In certain embodiments, the parental GPCR is a liganded-orphan GPCR or an altered liganded orphan GPCR.

In certain embodiments, the ligand upregulatable GPCR comprises a reporter polypeptide fused in-frame with the GURC-substituted parental GPCR. In certain embodiments, the reporter protein is luciferase, e.g. wild type or modified Renilla luciferase.

### Example 9

### GPCR Activation Assays

Receptor Expression: Mammalian cells or melanophores are used to recombinantly express GPCRs. The recombinantly expressed GPCR may be a native GPCR or a constitutively activated mutant of a native GPCR. In certain embodiments, the mammalian cell is an HEK-293 cell, an HEK-293T cell, a CHO cell, or a COS-7 cell.

Transient Transfection: On day one, 6×10⁶/10 cm dish of HEK293 cells well were plated out. On day two, two reaction tubes were prepared (the proportions to follow for each tube are per plate): tube A was prepared by mixing 4 μg DNA (e.g., pCMV vector; pCMV vector with receptor cDNA, etc.) in 0.5 ml serum free DMEM (Gibco BRL); tube B was prepared by mixing 24 μl lipofectamine (Gibco BRL) in 0.5 ml serum free DMEM. Tubes A and B were admixed by inversions (several times), followed by incubation at room temperature for 30-45 min. The admixture is referred to as the “transfection mixture”. Plated HEK293 cells were washed with 1XPBS, followed by addition of 5 ml serum free DMEM. 1 ml of the transfection mixture was added to the cells, followed by incubation for 4 hrs at 37° C./5% CO₂. The transfection mixture was removed by aspiration, followed by the addition of 10 ml of DMEM/10% Fetal Bovine Serum. Cells were incubated at 37° C./5% CO₂. After 48 hr incubation, cells were harvested and utilized for analysis.

Approximately 12×10⁶ HEK293 cells are plated on a 15 cm tissue culture plate. Grown in DME High Glucose Medium containing ten percent fetal bovine serum and one percent sodium pyruvate, L-glutamine, and antibiotics. Twenty-four hours following plating of HEK293 cells (or to 80% confluency), the cells are transfected using 12 μg of DNA. The 12 μg of DNA is combined with 60 μl of lipofectamine and 2 mL of DME High Glucose Medium without serum. The medium is aspirated from the plates and the cells are washed once with medium without serum. The DNA, lipofectamine, and medium mixture are added to the plate along with 10 mL of medium without serum. Following incubation at 37 degrees Celsius for four to five hours, the medium is aspirated and 25 ml of medium containing serum is added. Twenty-four hours following transfection, the medium is aspirated again, and fresh medium with serum is added. Forty-eight hours following transfection, the medium is aspirated and medium with serum is added containing geneticin (G418 drug) at a final concentration of 500 μg/mL. The transfected cells now undergo selection for positively transfected cells containing the G418 resistant gene. The medium is replaced every four to five days as selection occurs. During selection, cells are grown to create stable pools, or split for stable clonal selection.

Membrane Binding Assays: [⁵S]GTPγS Assay: When a G protein-coupled receptor is in its active state, either as a result of ligand binding or constitutive activation, the receptor couples to a G protein and stimulates the release of GDP and subsequent binding of GTP to the G protein. The alpha subunit of the G protein-receptor complex acts as a GTPase and slowly hydrolyzes the GTP to GDP, at which point the receptor normally is deactivated. Activated receptors continue to exchange GDP for GTP. The non-hydrolyzable GTP analog, [³⁵S]GTPγS, can be utilized to demonstrate enhanced binding of [³⁵S]GTPγS to membranes expressing activated receptors. The advantage of using [³⁵S]GTPγS binding to measure activation is that: (a) it is generically applicable to all G protein-coupled receptors; (b) it is proximal at the membrane surface making it less likely to pick-up molecules which affect the intracellular cascade.

The assay utilizes the ability of G protein coupled receptors to stimulate [³⁵S]GTPγS binding to membranes expressing the relevant receptors. The assay can, therefore, be used in the direct identification method to screen candidate compounds to endogenous GPCRs and non-endogenous, constitutively activated GPCRs. The assay is generic and has application to drug discovery at all G protein-coupled receptors.

The [³⁵S]GTPγS assay is incubated in 20 mM HEPES and between 1 and about 20 mM MgCl₂ (this amount can be adjusted for optimization of results, although 20 mM is preferred) pH 7.4, binding buffer with between about 0.3 and about 1.2 nM [³⁵S]GTPγS (this amount can be adjusted for optimization of results, although 1.2 is preferred) and 12.5 to 75 μg membrane protein (e.g, HEK293 cells expressing the Gs Fusion Protein; this amount can be adjusted for optimization) and 10 μM GDP (this amount can be changed for optimization) for 1 hour. Wheatgerm agglutinin beads (25 μl; Amersham) are then added and the mixture incubated for another 30 minutes at room temperature. The tubes are then centrifuged at 1500 x g for 5 minutes at room temperature and then counted in a scintillation counter.

Adenylyl Cyclase A Flash Plate™ Adenylyl Cyclase kit (New England Nuclear; Cat. No. SMP004A) designed for cell-based assays can be modified for use with crude plasma membranes. The Flash Plate wells can contain a scintillant coating which also contains a specific antibody recognizing cAMP. The cAMP generated in the wells can be quantitated by a direct competition for binding of radioactive cAMP tracer to the cAMP antibody. The following serves as a brief protocol for the measurement of changes in cAMP levels in whole cells that express the receptors.

Transfected cells were harvested approximately twenty four hours after transient transfection. Media is carefully aspirated off and discarded. 10 ml of PBS is gently added to each dish of cells followed by careful aspiration. 1 ml of Sigma cell dissociation buffer and 3 ml of PBS are added to each plate. Cells were pipetted off the plate and the cell suspension was collected into a 50 ml conical centrifuge tube. Cells were then centrifuged at room temperature at 1,100 rpm for 5 min. The cell pellet was carefully re-suspended into an appropriate volume of PBS (about 3 ml/plate). The cells were then counted using a hemocytometer and additional PBS was added to give the appropriate number of cells (with a final volume of about 50 μl/well).

cAMP standards and Detection Buffer (comprising 1 μCi of tracer [¹²⁵I cAMP (50 μl] to 11 ml Detection Buffer) was prepared and maintained in accordance with the manufacturer's instructions. Assay Buffer was prepared fresh for screening and contained 50 μl of Stimulation Buffer, 3 ul of test compound (12 μM final assay concentration) and 50 μl cells, Assay Buffer was stored on ice until utilized. The assay was initiated by addition of 50 μl of cAMP standards to appropriate wells followed by addition of 50 ul of PBSA to wells H-11 and H12. 50 μl of Stimulation Buffer was added to all wells. DMSO (or selected candidate compounds) was added to appropriate wells using a pin tool capable of dispensing 3 μl of compound solution, with a final assay concentration of 12 μM test compound and 100 μl total assay volume. The cells were then added to the wells and incubated for 60 min at room temperature. 100 μl of Detection Mix containing tracer cAMP was then added to the wells. Plates were then incubated additional 2 hours followed by counting in a Wallac MicroBeta scintillation counter. Values of cAMP/well were then extrapolated from a standard cAMP curve which was contained within each assay plate.

Cell-Based cAMP for Gi Coupled Target GPCRs: TSHR is a Gs coupled GPCR that causes the accumulation of cAMP upon activation. TSHR will be constitutively activated by mutating amino acid residue 623 (i.e., changing an alanine residue to an isoleucine residue). A Gi coupled receptor is expected to inhibit adenylyl cyclase, and, therefore, decrease the level of cAMP production, which can make assessment of cAMP levels challenging. An effective technique for measuring the decrease in production of cAMP as an indication of constitutive activation of a Gi coupled receptor can be accomplished by co-transfecting, most preferably, non-endogenous, constitutively activated TSHR (TSHR-A623I) (or an endogenous, constitutively active Gs coupled receptor) as a “signal enhancer” with a Gi linked target GPCR to establish a baseline level of cAMP. Upon creating a non-endogenous version of the Gi coupled receptor, this non-endogenous version of the target GPCR is then co-transfected with the signal enhancer, and it is this material that can be used for screening. We will utilize such approach to effectively generate a signal when a cAMP assay is used; this approach is preferably used in the direct identification of candidate compounds against Gi coupled receptors. It is noted that for a Gi coupled GPCR, when this approach is used, an inverse agonist of the target GPCR will increase the cAMP signal and an agonist will decrease the cAMP signal.

On day one, 2×10⁴ HEK293 cells/well will be plated out. On day two, two reaction tubes will be prepared (the proportions to follow for each tube are per plate): tube A will be prepared by mixing 2 μg DNA of each receptor transfected into the mammalian cells, for a total of 4 μg DNA (e.g., pCMV vector; pCMV vector with mutated THSR (TSHR-A623I); TSHR-A623I and GPCR, etc.) in 1.2 ml serum free DMEM (Irvine Scientific, Irvine, Calif.); tube B will be prepared by mixing 120 μl lipofectamine (Gibco BRL) in 1.2 ml serum free DMEM. Tubes A and B will then be admixed by inversions (several times), followed by incubation at room temperature for 30-45 min. The admixture is referred to as the “transfection mixture”. Plated HEK293 cells will be washed with 1XPBS, followed by addition of 10 ml serum free DMEM. 2.4 ml of the transfection mixture will then be added to the cells, followed by incubation for 4 hrs at 37° C./5% CO2. The transfection mixture will then be removed by aspiration, followed by the addition of 25 ml of DMEM/10% Fetal Bovine Serum. Cells will then be incubated at 37° C./5% CO2. After 24 hr incubation, cells will then be harvested and utilized for analysis.

A Flash Plate™ Adenylyl Cyclase kit (New England Nuclear; Cat. No. SMP004A) is designed for cell-based assays, however, can be modified for use with crude plasma membranes depending on the need of the skilled artisan. The Flash Plate wells will contain a scintillant coating which also contains a specific antibody recognizing cAMP. The cAMP generated in the wells can be quantitated by a direct competition for binding of radioactive cAMP tracer to the cAMP antibody. The following serves as a brief protocol for the measurement of changes in cAMP levels in whole cells that express the receptors.

Transfected cells will be harvested approximately twenty four hours after transient transfection. Media will be carefully aspirated off and discarded. 10 ml of PBS will be gently added to each dish of cells followed by careful aspiration. 1 ml of Sigma cell dissociation buffer and 3 ml of PBS will be added to each plate. Cells will be pipetted off the plate and the cell suspension will be collected into a 50 ml conical centrifuge tube.

Cells will then be centrifuged at room temperature at 1,100 rpm for 5 min. The cell pellet will be carefully re-suspended into an appropriate volume of PBS (about 3 ml/plate). The cells will then be counted using a hemocytometer and additional PBS is added to give the appropriate number of cells (with a final volume of about 50 μl/well).

cAMP standards and Detection Buffer (comprising 1 μCi of tracer [125I cAMP (50 μl] to 11 ml Detection Buffer) will be prepared and maintained in accordance with the manufacturer's instructions. Assay Buffer should be prepared fresh for screening and contained 50 μl of Stimulation Buffer, 3 μl of test compound (12 μM final assay concentration) and 50 μl cells, Assay Buffer can be stored on ice until utilized. The assay can be initiated by addition of 50 μl of cAMP standards to appropriate wells followed by addition of 50 μl of PBSA to wells H-11 and H12. Fifty ill of Stimulation Buffer will be added to all wells. Selected compounds (e.g., TSH) will be added to appropriate wells using a pin tool capable of dispensing 3 μl of compound solution, with a final assay concentration of 12 μM test compound and 100 μl total assay volume. The cells will then be added to the wells and incubated for 60 min at room temperature. 100 μl of Detection Mix containing tracer cAMP will then be added to the wells. Plates were then incubated additional 2 hours followed by counting in a Wallac MicroBeta scintillation counter. Values of cAMP/well will then be extrapolated from a standard cAMP curve which is contained within each assay plate.

Reporter-Based Assays: Cre-Luc Reporter Assay (Gs-associated receptors): HEK293 and HEK293T cells are plated-out on 96 well plates at a density of 2×104 cells per well and were transfected using Lipofectamine Reagent (BRL) the following day according to manufacturer instructions. A DNA/lipid mixture is prepared for each 6-well transfection as follows: 260 ng of plasmid DNA in 100 μl of DMEM were gently mixed with 2 μl of lipid in 100 μl of DMEM (the 260 ng of plasmid DNA consisted of 200 ng of a 8xCRE-Luc reporter plasmid, 50 ng of pCMV comprising endogenous receptor or non-endogenous receptor or pCMV alone, and long of a GPRS expression plasmid (GPRS in pcDNA3 (Invitrogen)). The 8XCRE-Luc reporter plasmid was prepared as follows: vector SRIF-β-gal was obtained by cloning the rat somatostatin promoter (−71/+51) at BglV-HindIII site in the pβgal-Basic Vector (Clontech). Eight (8) copies of cAMP response element were obtained by PCR from an adenovirus template AdpCF126CCRE8 (see, 7 Human Gene Therapy 1883 (1996)) and cloned into the SRIF-β-gal vector at the Kpn-BglV site, resulting in the 8xCRE-β-gal reporter vector. The 8xCRE-Luc reporter plasmid was generated by replacing the beta-galactosidase gene in the 8xCRE-β-gal reporter vector with the luciferase gene obtained from the pGL3-basic vector (Promega) at the HindIII-BamHI site. Following 30 min. incubation at room temperature, the DNA/lipid mixture was diluted with 400 μl of DMEM and 100 μl of the diluted mixture was added to each well. 100 μl of DMEM with 10% FCS were added to each well after a 4 hr incubation in a cell culture incubator. The following day the transfected cells were changed with 200 μl/well of DMEM with 10% FCS. Eight (8) hours later, the wells were changed to 100 μg/well of DMEM without phenol red, after one wash with PBS. Luciferase activity were measured the next day using the LucLite™ reporter gene assay kit (Packard) following manufacturer instructions and read on a 1450 MicroBeta™ scintillation and luminescence counter (Wallac).

AP1 reporter assay (Gq-associated receptors) A method to detect Gq stimulation depends on the known property of Gq-dependent phospholipase C to cause the activation of genes containing AP1 elements in their promoter. A Pathdetect™ AP-1 cis-Reporting System (Stratagene, Catalogue #219073) can be utilized following the protocol set forth above with respect to the CREB reporter assay, except that the components of the calcium phosphate precipitate were 410 ng pAP1-Luc, 80 ng pCMV-receptor expression plasmid, and 20 ng CMV-SEAP.

Srf-Luc Reporter Assay (Gq- associated receptors): One method to detect Gq stimulation depends on the known property of Gq-dependent phospholipase C to cause the activation of genes containing serum response factors in their promoter. A Pathdetect™ SRF-Luc-Reporting System (Stratagene) can be utilized to assay for Gq coupled activity in, e.g., COS7 cells. Cells are transfected with the plasmid components of the system and the indicated expression plasmid encoding endogenous or non-endogenous GPCR using a Mammalian Transfection™ Kit (Stratagene, Catalogue #200285) according to the manufacturer's instructions. Briefly, 410 ng SRF-Luc, 80 ng pCMV-receptor expression plasmid and 20 ng CMV-SEAP (secreted alkaline phosphatase expression plasmid; alkaline phosphatase activity is measured in the media of transfected cells to control for variations in transfection efficiency between samples) are combined in a calcium phosphate precipitate as per the manufacturer's instructions. Half of the precipitate is equally distributed over 3 wells in a 96-well plate, kept on the cells in a serum free media for 24 hours. The last 5 hours the cells are incubated with a selected compound. Cells are then lysed and assayed for luciferase activity using a Luclite™ Kit (Packard, Cat. # 6016911) and “Trilux 1450 Microbeta” liquid scintillation and luminescence counter (Wallac) as per the manufacturer's instructions. The data can be analyzed using GraphPad Prism™ 2.0a (GraphPad Software Inc.)

Intracellular IP₃ Accumulation Assay (Gq-associated receptors): On day 1, cells comprising the receptors (endogenous and/or non-endogenous) can be plated onto 24 well plates, usually 1×10⁵ cells/well (although his umber can be optimized. On day 2 cells can be transfected by firstly mixing 0.25 μg DNA in 50 μl serum free DMEM/well and 2 μlipofectamine in 50 μl serumfree DMEM/well. The solutions are gently mixed and incubated for 15-30 min at room temperature. Cells are washed with 0.5 ml PBS and 400 μl of serum free media is mixed with the transfection media and added to the cells. The cells are then incubated for 3-4 hrs at 37° C./5%CO₂ and then the transfection media is removed and replaced with 1 ml/well of regular growth media. On day 3 the cells are labeled with ³H-myo-inositol. Briefly, the media is removed and the cells are washed with 0.5 ml PBS. Then 0.5 ml inositol-free/serum free media (GIBCO BRL) is added/well with 0.25 μCi of ³H-myo-inositol/well and the cells are incubated for 16-18 hrs o/n at 37° C./5%CO₂. On Day 4 the cells are washed with 0.5 ml PBS and 0.45 ml of assay medium is added containing inositol-free/serum free media 10 μM pargyline 10 mM lithium chloride or 0.4 ml of assay medium and 50 μl of 10x ketanserin (ket) to final concentration of 10 μM. The cells are then incubated for 30 min at 37° C. The cells are then washed with 0.5 ml PBS and 200 μl of fresh/ice cold stop solution (1 M KOH; 18 mM Na-borate; 3.8 mM EDTA) is added/well. The solution is kept on ice for 5-10 min or until cells were lysed and then neutralized by 200 μl of fresh/ice cold neutralization sol. (7.5% HCL). The lysate is then transferred into 1.5 ml eppendorf tubes and 1 ml of chloroform/methanol (1:2) is added/tube. The solution is vortexed for 15 sec and the upper phase is applied to a Biorad AG1-X8™ anion exchange resin (100-200 mesh). Firstly, the resin is washed with water at 1:1.25 W/V and 0.9 ml of upper phase is loaded onto the column. The column is washed with 10 mls of 5 mM myo-inositol and 10 ml of 5 mM Na-borate/60 mM Na-formate. The inositol tris phosphates are eluted into scintillation vials containing 10 ml of scintillation cocktail with 2 ml of 0.1 M formic acid/1 M ammonium formate. The columns are regenerated by washing with 10 ml of 0.1 M formic acid/3 M ammonium formate and rinsed twice with dd H₂O and stored at 4° C. in water.

Fluorometric Imaging Plate Reader (FLIPR) Assay for the Measurement of Intracellular Calcium Concentration: Target Receptor (experimental) and pCMV (negative control) stably transfected cells from respective clonal lines are seeded into poly-D-lysine pretreated 96-well plates (Becton-Dickinson, #356640) at 5.5×10⁴ cells/well with complete culture medium (DMEM with 10% FBS, 2 mM L-glutamine, 1 mM sodium pyruvate) for assay the next day. To prepare Fluo4-AM (Molecular Probe, #F14202) incubation buffer stock, 1 mg Fluo4-AM is dissolved in 467 μl DMSO and 467 μl Pluoronic acid (Molecular Probe, #P3000) to give a 1 mM stock solution that can be stored at −20° C. for a month. Fluo4-AM is a fluorescent calcium indicator dye.

Candidate compounds are prepared in wash buffer (1X HBSS/2.5 mM Probenicid/20 mM HEPES at pH 7.4).

At the time of assay, culture medium is removed from the wells and the cells are loaded with 100 μl of 4 μM Fluo4-AM/2.5 mM Probenicid (Sigma, #P8761)/20 mM HEPES/complete medium at pH 7.4. Incubation at 37° C./5% CO₂ is allowed to proceed for 60 min.

After the 1 hr incubation, the Fluo4-AM incubation buffer is removed and the cells are washed 2X with 100 μl wash buffer. In each well is left 100 μl wash buffer. The plate is returned to the incubator at 37° C./5% CO₂ for 60 min.

FLIPR (Fluorometric Imaging Plate Reader; Molecular Device) is programmed to add 50 μl candidate compound on the 30^{th} second and to record transient changes in intracellular calcium concentration ([Ca²⁺]) evoked by the candidate compound for another 150 seconds. Total fluorescence change counts are used to determine agonist activity using the FLIPR software. The instrument software normalizes the fluorescent reading to give equivalent initial readings at zero.

In some embodiments, the cells comprising Target Receptor further comprise promiscuous G alpha 15/16 or a chimeric Gq/Gi alpha unit.

Although the foregoing provides a FLIPR assay for agonist activity using stably transfected cells, a person of ordinary skill in the art would readily be able to modify the assay in order to characterize antagonist activity. Said person of ordinary skill in the art would also readily appreciate that, alternatively, transiently transfected cells could be used.

It is evident from the above results and discussion that the subject invention provides an important new means for making a ligand-upregulatable GPCR. In particular, the subject invention provides a system for screening chemical agent libraries to find GPCR modulators. As such, the subject methods and systems find use in a variety of different applications, including research, medical, therapeutic and other applications. Accordingly, the present invention represents a significant contribution to the art.

Applicant reserves the right to exclude any one or more native GPCR, parental GPCR, or ligand upregulatable GPCR from any of the embodiments of the invention. Applicant further reserves the right to exclude any polynucleotide or polypeptide from any of the embodiments of the invention.

1. A method for making a ligand upregulatable GPCR, said method comprising the step of providing a substituted GPCR by modifying the TM5-IC3-TM6 segment of a parental GPCR such that the TM5-IC3-TM6 segment comprises substitution of the amino acid sequence of a GPCR upregulating cassette.

2. A method according to claim 1 wherein the parental GPCR is a native or altered known GPCR or a native or altered orphan GPCR.

3. A method according to claim 1 wherein the parental GPCR comprises an operably linked reporter protein.

4. A method according to claim 1 further comprising the steps of:
(a) producing said substituted GPCR in a host cell; and
(b) comparing a detectable level of said substituted GPCR in the presence of a ligand to a detectable level of said substituted GPCR in the absence of the ligand;
wherein a substituted GPCR that is detectable at a higher level in the host cell in the presence of the ligand than in the absence of the ligand is a ligand upregulatable GPCR.

5. A method according to claim 4 wherein the parental GPCR is a native or altered known GPCR or a native or altered liganded-orphan GPCR.

6. A method according to claim 4 wherein the parental GPCR comprises an operably linked reporter protein.

7. A method for making a nucleic acid encoding a ligand upregulatable GPCR, said method comprising the step of providing a nucleic acid encoding a substituted GPCR by modifying the TM5-IC3-TM6 segment-encoding nucleic acid of a parental GPCR-encoding nucleic acid such that the TM5-IC3-TM6 segment comprises substitution of the nucleotide sequence of a GPCR upregulating cassette.

8. A method according to claim 7 wherein the parental GPCR is a native or altered known GPCR or a native or altered orphan GPCR.

9. A method according to claim 7 wherein the parental GPCR comprises an operably linked reporter protein.

10. A method according to claim 7 further comprising the steps of:
(a) producing said substituted GPCR in a host cell; and,
(b) comparing a detectable level of said substituted GPCR in the presence of a ligand to a detectable level of said substituted GPCR in the absence of the ligand;
wherein a nucleic acid encoding a substituted GPCR that is detectably present at higher level in the host cell in the presence of the ligand than in the absence of the ligand is a nucleic acid encoding a ligand upregulatable GPCR.

11. A method according to claim 10 wherein the parental GPCR is a native or altered known GPCR or a native or altered liganded-orphan GPCR.

12. A method according to claim 10 wherein the parental GPCR comprises an operably linked reporter protein.

13. A method of identifying whether a test compound is a ligand for a parental GPCR, said method comprising the steps of:
(a) contacting the test compound with a ligand upregulatable GPCR according to any one of claims 1 to 6, which ligand upregulatable GPCR is expressed by a host cell; and
(b) comparing a first detectable level of said ligand upregulatable GPCR in the presence of the test compound to a second detectable level of said ligand upregulatable GPCR in the absence of the test compound;
wherein said first detectable level greater than said second detectable level indicates that the test compound is a ligand for the parental GPCR.

14. The method of claim 13, wherein said host cell comprises an expression vector comprising a polynucleotide encoding the ligand upregulatable GPCR.

15. A method of preparing a composition which comprises identifying a ligand of a GPCR and then admixing a carrier and the ligand, wherein the ligand is identified by a method according to claim 13.

16. A method according to claim 15 wherein said ligand is a modulator of the GPCR.

17. A method of modulating a GPCR, said method comprising:
contacting a ligand for the GPCR identified according to the method of claim 13 with the GPCR, wherein said ligand is a modulator of the GPCR.

18. A method for treating an individual for a GPCR-related disorder, said method comprising:
administering to said individual an effective amount of a ligand for the GPCR, wherein said ligand is identified according to the method of claim 13 and wherein said ligand is a modulator of the GPCR.

19. The method of any one of claims 1 to 6, wherein the parental GPCR is not an adrenergic receptor (adrenoreceptor) or variant thereof.

20. The method of any one of claims 7 to 12, wherein the parental GPCR is not an adrenergic receptor (adrenoreceptor) or variant thereof.

21. The method of claim 13, wherein the parental GPCR is not an adrenergic receptor (adrenoreceptor) or variant thereof.

22. The method of claim 14, wherein the parental GPCR is not an adrenergic receptor (adrenoreceptor) or variant thereof.

23. The method of claim 13, further comprising the step of formulating the ligand into a pharmaceutical composition.

24. The method of claim 14, further comprising the step of formulating the ligand into a pharmaceutical composition.

25. The method of claim 21, further comprising the step of formulating the ligand into a pharmaceutical composition.

26. The method of claim 22, further comprising the step of formulating the ligand into a pharmaceutical composition.
